(19) United States
(12) Patent Application Publication
(10) Pub. No.: US 2025/0017107 A1
(43) Pub. Date: Jan. 9, 2025

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Kouitsu SASAKI, Kanagawa (JP); Masaki MORITA, Kanagawa (JP); Saika IZUMI, Kanagawa (JP); Yasunori YONEKUTA, Kanagawa (JP); Ryoji GOTO, Kanagawa (JP)

(73) Assignee: FUJIFILM Corporation, Tokyo (JP)

(21) Appl. No.: 18/883,954

(22) Filed: Sep. 12, 2024

Related U.S. Application Data
(63) Continuation of application No. PCT/JP2023/011967, filed on Mar. 24, 2023.

(30) Foreign Application Priority Data
Mar. 30, 2022 (JP) 2022-055868

(51) Int. Cl. H10K 85/60; C07C 225/22; C07D 209/96; C07D 263/57; C07D 409/06; C07D 487/04; C07D 513/04; H10K 30/30; H10K 85/20

(52) U.S. Cl. CPC H10K 85/6572; C07C 225/22; C07D 209/96; C07D 263/57; C07D 409/06; C07D 487/04; C07D 513/04; H10K 85/211; H10K 85/623; H10K 85/633; H10K 85/636; H10K 85/654; H10K 85/657; H10K 30/30; H10K 85/615; H10K 85/655

(57) ABSTRACT

A first object of the present invention is to provide a photoelectric conversion element having excellent manufacturing suitability. A second object of the present invention is to provide an imaging element, an optical sensor, and a compound.

The photoelectric conversion element includes a conductive film, a photoelectric conversion film, and a transparent conductive film in this order, in which the photoelectric conversion film contains one of more of compounds represented by any of Formula (1) to (6).

### CROSS-REFERENCE TO RELATED APPLICATIONS

This application is a Continuation of PCT International Application No. PCT/JP2023/011967 filed on Mar. 24, 2023, which claims priority under 35 U.S.C. § 119(a) to Japanese Patent Application No. 2022-055868 filed on Mar. 30, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, the development of an element (for example, an imaging element) having a photoelectric conversion film has been progressing.

For example, it is disclosed in WO2020/013246A that a compound described below is used as a material applied to a photoelectric conversion element.

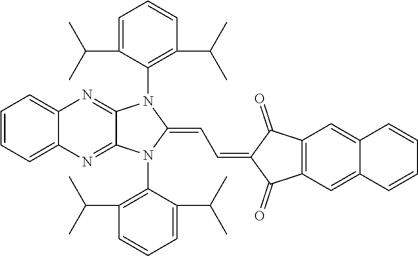 [SMILES: CC(C)c1cccc(C(C)C)c1N1C(=CC=C2C(=O)c3cc4ccccc4cc3C2=O)N(c2c(C(C)C)cccc2C(C)C)c2nc3ccccc3nc21]

### SUMMARY OF THE INVENTION

In recent years, along with the demand for improving the performance of imaging elements, optical sensors, and the like, further improvements are required for various characteristics required for photoelectric conversion elements used therein.

As a result of producing and studying a photoelectric conversion element using the compound disclosed in WO2020/013246A, the present inventors have found that the photoelectric conversion efficiency (external quantum efficiency) of the photoelectric conversion element may vary depending on a film formation rate (for example, a vapor deposition rate) in a case of manufacturing the photoelectric conversion film. That is, it has been clarified that there is a room for studying a photoelectric conversion element in which the photoelectric conversion efficiency (external quantum efficiency) does not depend on the film formation rate of the photoelectric conversion film (in other words, the manufacturing suitability is excellent).

Therefore, an object of the present invention is to provide a photoelectric conversion element having excellent manufacturing suitability.

Another object of the present invention is to provide an imaging element, an optical sensor, and a compound.

The inventors of the present invention have conducted extensive studies on the above-described object. As a result, the inventors have found that it is possible to solve the above-described problems by applying the compound having a predetermined structure to the photoelectric conversion film, and have completed the present invention.
[1] A photoelectric conversion element comprising in the following order, a conductive film, a photoelectric conversion film, and a transparent conductive film, in which the photoelectric conversion film contains one or more of compounds represented by Formulae (1) to (6).
[2] The photoelectric conversion element according to [1], in which the compound represented by the Formula (1) is a compound represented by Formula (1A).
[3] The photoelectric conversion element according to [1] or [2], in which the compound represented by the Formula (1) is a compound represented by Formula (1B).
[4] The photoelectric conversion element according to any one of [1] to [3], in which the compound represented by Formula (1) is a compound represented by Formula (1C).
[5] The photoelectric conversion element according to any one of [1] to [4], in which the photoelectric conversion film contains one or more of the compounds represented by any of Formulae (1) to (3).
[6] The photoelectric conversion element according to any one of [1] to [5], in which the aromatic ring group represented by Formula (X) represents an aromatic ring group represented by Formula (X′).
[7] The photoelectric conversion element according to any one of [1] to [6], in which the photoelectric conversion film further contains an n-type organic semiconductor, and the photoelectric conversion film has a bulk hetero structure formed in a state where one or more of the compounds represented by any of Formulae (1) to (6) and the n-type organic semiconductor are mixed with each other.
[8] The photoelectric conversion element according to [7], in which the n-type organic semiconductor includes fullerenes selected from the group consisting of a fullerene and a derivative of a fullerene.
[9] The photoelectric conversion element according to any one of [1] to [8], further comprising one or more interlayers between the conductive film and the transparent conductive film, in addition to the photoelectric conversion film.
[10] An imaging element comprising the photoelectric conversion element according to any one of [1] to [9].
[11] The imaging element according to [10], further comprising another photoelectric conversion element that receives light having a wavelength different from a wavelength of light received by the photoelectric conversion element.
[12] The imaging element according to [11], in which the photoelectric conversion element and the other photoelectric conversion element are laminated, and at least a part of incidence ray is transmitted through the photoelectric conversion element, and then is received by the other photoelectric conversion element.
[13] The imaging element according to [11] or [12], in which the photoelectric conversion element is a green photoelectric conversion element, and the other photoelectric conversion element includes a blue photoelectric conversion element and a red photoelectric conversion element.
[14] An optical sensor comprising the photoelectric conversion element according to any one of [1] to [9].
[15] A compound represented by Formula (1).

According to the present invention, it is possible to provide a photoelectric conversion element having excellent manufacturing suitability.

In addition, according to the present invention, it is possible to provide the imaging element, the optical sensor, and the compound.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

FIG. 3 is a schematic cross-sectional view of an embodiment of an imaging element.

### DESCRIPTION OF THE PREFERRED EMBODIMENTS

Hereinafter, suitable embodiments of a photoelectric conversion element of the present invention will be described.

In the present specification, the numerical range represented by “to” means a range including numerical values denoted before and after “to” as a lower limit value and an upper limit value.

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a hydrogen atom may be a light hydrogen atom (an ordinary hydrogen atom) or a deuterium atom (for example, a double hydrogen atom and the like).

In the present specification, examples of the halogen atom include a fluorine atom, a chlorine atom, a bromine atom, and an iodine atom.

In the present specification, a “substituent” includes the group exemplified by a substituent W described later, unless otherwise specified.

### (Substituent W)

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (may also be referred to as a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a silyl group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, unless otherwise specified, the number of carbon atoms of the alkyl group is preferably 1 to 20, more preferably 1 to 12, still more preferably 1 to 10, and particularly preferably 1 to 6.

The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be, for example, a cycloalkyl group, a bicycloalkyl group, or a tricycloalkyl group, and may have a cyclic structure thereof as a partial structure.

In the alkyl group which may have a substituent, a substituent which may be contained in the alkyl group is not particularly limited, an example thereof includes the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The number of carbon atoms of the above-described alkenyl group is preferably 2 to 20, more preferably 2 to 12, still more preferably 2 to 6, and particularly preferably 2 or 3. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The number of carbon atoms of the alkynyl group is preferably 2 to 20, more preferably 2 to 12, still more preferably 2 to 6, and particularly preferably 2 or 3. In the alkynyl group which may have a substituent, the substituent which may be contained in the alkynyl group includes the same examples as the substituent in the alkyl group which may have a substituent.

In the present specification, unless otherwise specified, examples of a silyl group which may have a substituent include a group represented by —Si(R^{S1})(R^{S2})(R^{S3}). R^{S1}, R^{S2}, and R^{S3} each independently represent a hydrogen atom or a substituent, and preferably represent an alkyl group which may have a substituent, an alkoxy group which may have a substituent, an alkylthio group which may have a substituent, an aryl group which may have a substituent, or a heteroaryl group which may have a substituent.

In the present specification, an aromatic ring may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the above-described aromatic ring is preferably 5 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the above-described heteroatoms include a nitrogen atom, a sulfur atom, an oxygen atom, a selenium atom, a tellurium atom, a phosphorus atom, a silicon atom, and a boron atom.

Examples of the above-described aromatic hydrocarbon ring include a benzene ring, a naphthalene ring, an anthracene ring, and a phenanthrene ring.

Examples of the above-described aromatic heterocyclic ring include a pyridine ring, a pyrimidine ring, a pyridazine ring, a pyrazine ring, a triazine ring (a 1,2,3-triazine ring, a 1,2,4-triazine ring, a 1,3,5-triazine ring, and the like), a tetrazine ring (a 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (a 5H-pyrrolo[1,2-a]imidazole ring and the like), an imidazooxazole ring (an imidazo[2,1-b]oxazole ring and the like), a thienothiazole ring (a thieno[2,3-d]thiazole ring and the like), a benzothiadiazole ring, a benzodithiophene ring (a benzo[1,2-b:4,5-b′]dithiophene ring and the like), a thienothiophene ring (a thieno[3,2-b]thiophene ring and the like), a thiazolothiazole ring (a thiazolo[5,4-d]thiazole ring and the like), a naphthodithiophene ring (a naphtho[2,3-b: 6,7-b′]dithiophene ring, a naphtho[2,1-b: 6,5-b′]dithiophene ring, a naphtho[1,2-b: 5,6-b′]dithiophene ring, a 1,8-dithiacyclopenta[b,g]naphthalene ring, and the like), a benzothienobenzothiophene ring, a dithieno[3,2-b: 2′,3′-d]thiophene ring, and a 3,4,7,8-tetrathiadicyclopenta[a,e]pentalene ring.

In the aromatic ring which may have a substituent, a type of the substituent that may be included in the aromatic ring is not particularly limited, and examples thereof include a substituent W. In a case where the aromatic ring has a substituent, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, a type of the substituents that may be included in these groups is not particularly limited, and examples thereof include a substituent W. In a case where these groups each of which may have a substituent have substituents, the number of substituents may be 1 or more (for example, 1 to 4 or the like).

In the present specification, in a case where a plurality of identical symbols indicating a type or the number of groups are present in Formula (General Formula), which indicates a chemical structure, contents of these plurality of identical symbols indicating a type or the number of groups are independent of each other, and the contents of the identical symbols may be the same or different from each other unless otherwise specified.

In the present specification, in a case where a plurality of identical groups (alkyl groups or the like) are present in one Formula (General Formula), which indicates a chemical structure, specific contents between these plurality of identical groups are independent of each other, and the specific contents between the plurality of identical groups may be the same or different from each other, unless otherwise specified.

A bonding direction of a divalent group (for example, —CO—O—) described in the present specification is not limited unless otherwise specified. For example, in a case where Y is —CO—O— in the compound represented by General Formula “X—Y—Z”, the above-described compound may be “X—O—CO—Z” or may be “X—CO—O—Z”.

In the present specification, regarding a compound that may have a geometric isomer (cis-trans isomer), a general formula or a structural formula representing the above compound may be described only in the form of either a cis isomer or a trans isomer for convenience. Even in such a case, unless otherwise specified, the form of the compound is not limited to either the cis isomer or the trans isomer, and the compound may be either the cis isomer or the trans isomer.

### [Photoelectric Conversion Element]

The photoelectric conversion element according to an embodiment of the present invention includes a conductive film, a photoelectric conversion film, and a transparent conductive film in this order, in which the photoelectric conversion film contains one or more compounds represented by any of Formulae (1) to (6) (hereinafter, referred to as a “specific compound”).

The photoelectric conversion element according to the embodiment of the present invention has excellent manufacturing suitability by the above-described configuration.

The mechanism capable of solving the above-described object by adopting such a configuration of the photoelectric conversion element according to the embodiment of the present invention is not always clear, but the present inventors have presumed as follows.

Examples of the main feature point of the specific compound include a point that the specific compound has a DA type (donor-acceptor type) structure and a group represented by Formula (X) at a predetermined position in the donor site of the specific compound.

In a photoelectric conversion film manufactured at a relatively fast film formation rate, disorder in the arrangement of molecules in the film is likely to occur as compared with a photoelectric conversion film manufactured at a relatively slow film formation rate. As a result, the photoelectric conversion element including the photoelectric conversion film manufactured at a relatively fast film formation rate tends to have a tendency that the charge transfer in the photoelectric conversion film is hindered by the disorder of the above-described molecular arrangement, and the photoelectric conversion efficiency (external quantum efficiency) is lowered. On the other hand, the specific compound has a group represented by Formula (X) which is a structure in which the x-conjugation is wide and the steric hindrance is large, and a structure in which the x-conjugated plane derived from the group represented by Formula (X) extends from the main skeleton. It is presumed that, due to the above-described structure, the specific compound can exist in the photoelectric conversion film with other specific compounds and a component having a x-conjugated plane, which can be optionally included, such as fullerene in a state where the x-conjugated planes are in close proximity to each other. As a result, it is presumed that the charge mobility is less likely to be impaired even in a case where the molecular arrangement in the film is disturbed because of the high-speed film formation.

Hereinafter, the fact that the manufacturing suitability of the photoelectric conversion element is more excellent, the fact that the photoelectric conversion efficiency of the photoelectric conversion element is more excellent, and/or the fact that the response speed of the photoelectric conversion element is more excellent is also referred to as “effects of the present invention are more excellent”.

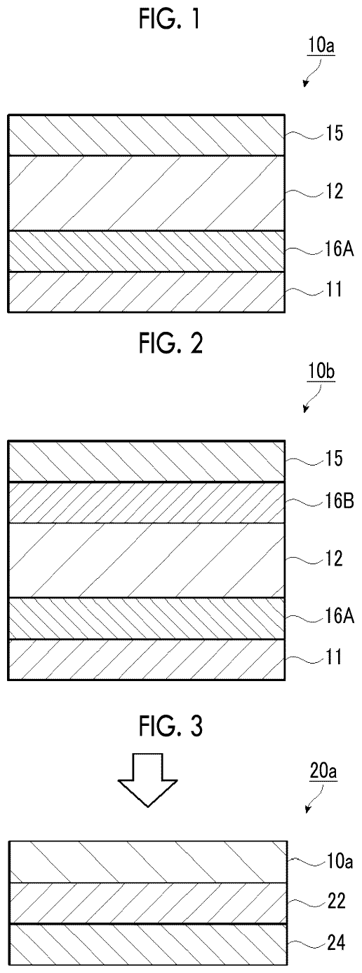

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound described later, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage is applied between the pair of electrodes.

The above-described voltage is preferably 1.0×10⁻⁵ to 1.0×10⁷ V/cm, and from the viewpoint of performance and power consumption, more preferably 1.0×10⁻⁴ to 1.0×10⁷ V/cm, and still more preferably 1.0×10⁻³ to 5.0×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### [Photoelectric Conversion Film]

The photoelectric conversion film is a film containing a specific compound.

The specific compound is a compound represented by any of Formulae (1) to (6).

Hereinafter, the compound represented by any of Formulae (1) to (6) will be described in detail.

### <Compound Represented by Formula (1)>

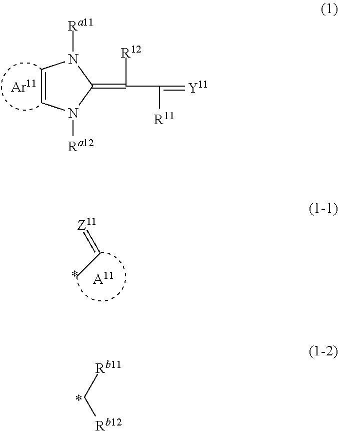

In Formula (1), Y¹¹ represents a group represented by Formula (1-1) or a group represented by Formula (1-2). From the viewpoint that the effect of the present invention is more excellent, Y¹¹ is preferably a group represented by Formula (1-1).

In Formula (1-1), A¹¹ represents a ring which contains at least two carbon atoms and may have a substituent. The two carbon atoms are intended as a carbon atom to which Z¹¹ in Formula (1-1) is bonded and a carbon atom adjacent to the carbon atom to which Z¹¹ is bonded, and the two carbon atoms are atoms that constitute A¹¹.

In addition, in the above-described ring, carbon atoms constituting the ring may be substituted with another carbonyl carbon (>C═O) and/or another thiocarbonyl carbon (>C═S). The other carbonyl carbon (>C═O) and the other thiocarbonyl carbon (>C═S) as used herein each mean a carbonyl carbon and a thiocarbonyl carbon each of which has a carbon atom other than the carbon atom bonded to Z¹¹ among the carbon atoms constituting the ring, as a constituent.

A¹¹ preferably has 3 to 30 carbon atoms, more preferably has 3 to 20 carbon atoms, and still more preferably has 3 to 15 carbon atoms. The number of carbon atoms described above includes two carbon atoms specified in Formula (1-1).

A¹¹ may have a heteroatom, and examples of the heteroatom include a nitrogen atom, a sulfur atom, an oxygen atom, a selenium atom, a tellurium atom, a phosphorus atom, a silicon atom, and a boron atom. Among these, the nitrogen atom, the sulfur atom, or the oxygen atom is preferable, and the oxygen atom is more preferable.

A¹¹ may have a substituent, and a halogen atom is preferable as the substituent.

A¹¹ preferably has 0 to 10 heteroatoms, more preferably has 0 to 5 heteroatoms, and still more preferably has 0 to 2 heteroatoms. The number of heteroatoms described above does not include the number of heteroatoms that the group represented by Z¹¹ in Formula (1-1) contains and the number of halogen atoms that A¹¹ can have as a substituent.

A¹¹ may or may not exhibit aromaticity.

A¹¹ may have a monocyclic structure or a fused ring structure, but is preferably a 5-membered ring, a 6-membered ring, or a fused ring containing at least any of a 5-membered ring or a 6-membered ring. The number of rings forming the above-described fused-ring is preferably 2 to 4, and more preferably 2 to 3.

The ring represented by A¹¹ preferably has a group represented by Formula (A1). *¹ represents a bonding position with a carbon atom to which Z¹¹ specified in Formula (1-1) is bonded, and *² represents a bonding position with a carbon atom adjacent to the carbon atom to which Z¹¹ specified in Formula (1-1) is bonded.

*¹-L-Y—Z—*² (A1)

In Formula (A1), L represents a single bond or —NR^{L}—.

R^{L} represents a hydrogen atom or a substituent.

The type of the substituent represented by R^{L} is not particularly limited, and among them, an alkyl group which may have a substituent, an aryl group which may have a substituent, or a heteroaryl group which may have a substituent is preferable.

L is preferably a single bond.

Y represents —CR^{Y1}=CR^{Y2}, —CS—NR^{Y3}, —CS—, —NR^{Y4}—, or —N═CR^{Y5}, and among these, —CR^{Y1}=CR^{Y2}— is preferable.

R^{Y1} to R^{Y5} each independently represent a hydrogen atom or a substituent.

The type of the substituent represented by R^{Y1} to R^{Y5} is not particularly limited, and among them, an alkyl group which may have a substituent, an aryl group which may have a substituent, or a heteroaryl group which may have a substituent is preferable.

In a case where Y represents —CR^{Y1}=CR^{Y2}—, R^{Y1} and R^{Y2} are preferably bonded to each other to form a ring, and R^{Y1} and R^{Y2} are more preferably bonded to each other to form a benzene ring.

Z represents a single bond, —CO—, —CS—, —C(═NR^{Z1})—, or —C(═CR^{Z2}R^{Z3})—, and among these, Z more preferably represents —CO— or —C(═CR^{Z2}R^{Z3})—, and still more preferably represents —CO—.

R^{Z1} represents a hydrogen atom or a substituent.

The type of a substituent represented by R^{Z1} is not particularly limited, and examples thereof include the group exemplified by the above-described substituent W.

From the viewpoint that the effect of the present invention is more excellent, R^{Z1} is preferably a hydrogen atom, an alkyl group which may have a substituent, an aryl group which may have a substituent, or a heteroaryl group which may have a substituent, and more preferably a hydrogen atom.

R^{Z2} and R^{Z3} each independently represent a cyano group or —COOR^{Z4}. R^{Z4} represents an alkyl group which may have a substituent, an aryl group which may have a substituent, or a heteroaryl group which may have a substituent.

Among these, R^{Z2} and R^{Z3} are each independently preferably a cyano group.

The combination of L, Y, and Z, which are described above, is preferably a combination of -L-Y—Z—, which is bonded to two carbon atoms specified in Formula (1-1) to form a ring that is a 5-membered ring or a 6-membered ring. In addition, as described above, the 5-membered ring or the 6-membered ring may be fused with a different ring (preferably a benzene ring) to form a fused ring structure.

Among these, the group represented by Formula (A1) is more preferably a group represented by Formula (A2).

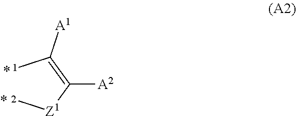

In Formula (A2), A¹ and A² each independently represent a hydrogen atom or a substituent.

A¹ and A² are preferably bonded to each other to form a ring, and A¹ and A² are more preferably bonded to each other to form a benzene ring.

The above-described benzene ring formed by A¹ and A² further preferably has a substituent. As the substituent, a halogen atom is preferable, and a chlorine atom or a fluorine atom is more preferable.

Substituents that the benzene ring formed by A¹ and A² has may be further bonded to each other to form a ring. For example, substituents that the benzene ring formed by A¹ and A² has may be further bonded to each other to form a benzene ring.

*¹, *², and Z¹ in Formula (A2) each have the same definitions as *¹, *², and Z in Formula (A1) described above, and the suitable embodiments thereof are also the same.

Among these, the group represented by Formula (A1) is still more preferably a group represented by Formula (A3).

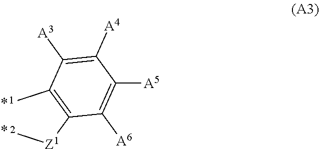

In Formula (A3), A³ to A⁶ each independently represent a hydrogen atom or a substituent. Among these, A³ to A⁶ are each independently preferably a hydrogen atom or a halogen atom, and more preferably a hydrogen atom, a chlorine atom, or a fluorine atom, and still more preferably a hydrogen atom.

A³ and A⁴ may be bonded to each other to form a ring, A⁴ and A⁵ may be bonded to each other to form a ring, and A⁵ and A⁶ may be bonded to each other to form a ring. Rings formed by each bonding A³ and A⁴, A⁴ and A⁵, and A⁵ and A⁶ are each preferably a benzene ring. Among these, A⁴ and A⁵ are preferably bonded to each other to form a ring, and the ring formed by bonding A⁴ and A⁵ to each other is preferably a benzene ring. The ring formed by bonding A⁴ and A⁵ to each other may be further substituted with a substituent.

*¹, *², and Z¹ in Formula (A3) each have the same definitions as *¹, *², and Z in Formula (A1), and the suitable embodiments thereof are also the same.

As such a ring, a merocyanine coloring agent usually used as an acidic nucleus is preferable, and specific examples thereof include as follows:
(a) 1,3-dicarbonyl nuclei: for example, a 1,3-indandione nucleus, 1,3-cyclohexanedione, 5,5-dimethyl-1,3-cyclohexanedione, 1,3-dioxane-4,6-dione, and the like;
(b) pyrazolinone nuclei: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like;
(c) isoxazolinone nuclei: for example, 3-phenyl-2-isoxazolin-5-one, 3-methyl-2-isoxazolin-5-one, and the like;
(d) oxindole nuclei: for example, 1-alkyl-2,3-dihydro-2-oxindole and the like;
(e) 2,4,6-trioxohexahydropyrimidine nuclei: for example, barbituric acid or 2-thibarbituric acid and derivatives thereof, and the like, and examples of the derivatives include 1-alkyl compounds such as 1-methyl and 1-ethyl, 1,3-dialkyl compounds such as 1,3-dimethyl, 1,3-diethyl, 1,3-dibutyl, 1,3-diaryl compounds such as 1,3-diphenyl, 1,3-di(p-chlorophenyl), 1,3-di(p-ethoxycarbonylphenyl), 1-alkyl-1-aryl compounds such as 1-ethyl-3-phenyl, 1,3-diheteroaryl compounds such as 1,3-di(2-pyridyl), and the like;
(f) 2-thio-2,4-thiazolidinedione nuclei: for example, rhodanine and derivatives thereof, and the like, and examples of the derivatives include 3-aklylrhodanine such as 3-methylrhodanine, 3-ethylrhodanine, 3-allylrhodanine, 3-arylrhodanine such as 3-phenylrhodanine, 3-heteroarylrhodanine such as 3-(2-pyridyl) rhodanine, and the like;
(g) 2-thio-2,4-oxazolidinedione (2-thio-2,4-(3H,5H)-oxazoledione) nuclei: for example, 3-ethyl-2-thio-2,4-oxazolidinedione, and the like;
(h) thianaphthenone nuclei: for example, 3 (2H)-thianaphthenone-1,1-dioxide, and the like;
(i) 2-thio-2,5-thiazolidinedione nuclei: for example, 3-ethyl-2-thio-2,5-thiazolidinedione, and the like;
(j) 2,4-thiazolidinedione nuclei: for example, 2,4-thiazolidinedione, 3-ethyl-2,4-thiazolidinedione, 3-phenyl-2,4-thiazolidinedione, and the like;
(k) thiazoliin-4-one nuclei: for example, 4-thiazolinone, 2-ethyl-4-thiazolinone, and the like;
(l) 2,4-imidazolidinedione (hydantoin) nuclei: for example, 2,4-imidazolidinedione, 3-ethyl-2,4-imidazolidinedione, and the like;
(m) 2-thio-2,4-imidazolidinedione (2-thiohydantoin) nuclei: for example, 2-thio-2,4-imidazolidinedione, 3-ethyl-2-thio-2,4-imidazolidinedione, and the like;
(n) imidazolin-5-one nuclei: for example, 2-propylmercapto-2-imidazolin-5-one, and the like;
(o) 3,5-pyrazolidinedione nuclei: for example, 1,2-diphenyl-3,5-pyrazolidinedione, 1,2-dimethyl-3,5-pyrazolidinedione, and the like;
(p) benzothiophen-3 (2H)-one nuclei: for example, benzothiophen-3 (2H)-one, oxobenzothiophen-3 (2H)-one, dioxobenzothiophen-3 (2H)-one, and the like;
(q) indanone nuclei: for example, 1-indanone, 3-phenyl-1-indanone, 3-methyl-1-indanone, 3,3-diphenyl-1-indanone, 3,3-dimethyl-1-indanone, and the like;
(r) benzofuran-3-(2H)-one nucleus: for example, benzofuran-3-(2H)-one, and the like; and
(s) 2,2-dihydrophenalene-1,3-dione nucleus, and the like.

Z¹¹ represents an oxygen atom, a sulfur atom, =NR^{ZT1}, or =CR^{ZT2}R^{ZT3}. From the viewpoint that the effect of the present invention is more excellent, among these, Z¹¹ is preferably an oxygen atom or =CR^{ZT2}R^{ZT3}, and more preferably an oxygen atom.

R^{ZT1} represents a hydrogen atom or a substituent.

The type of the substituent represented by R^{ZT1} is not particularly limited, and examples thereof include the group exemplified by the above-described substituent W.

From the viewpoint that the effect of the present invention is more excellent, R^{ZT1} is preferably a hydrogen atom, an alkyl group which may have a substituent, an aryl group which may have a substituent, or a heteroaryl group which may have a substituent, and more preferably a hydrogen atom.

R^{ZT2} and R^{ZT3} each independently represent a cyano group or —COOR^{ZT4}. R^{ZT4} represents an alkyl group which may have a substituent, an aryl group which may have a substituent, or a heteroaryl group which may have a substituent.

Among these, R^{ZT2} and R^{ZT3} are each preferably a cyano group, from the viewpoint that the effect of the present invention is more excellent.

In Formula (1-2), R^{b11} and R^{b12} each independently represent a cyano group or —COOR^{B1}. R^{B1} represents an alkyl group which may have a substituent, an aryl group which may have a substituent, or a heteroaryl group which may have a substituent.

* represents a bonding position.

In Formula (1), R¹¹ and R¹² each independently represent a hydrogen atom or a substituent.

The type of the substituent represented by R¹¹ and R¹² is not particularly limited, and examples thereof include the group exemplified by the above-described substituent W.

It is preferable that R¹¹ and R¹² represent a hydrogen atom.

R^{a11} and R^{a12} each independently represent an aromatic ring group which may have a substituent, or —C(R^{L11})(R^{L12})(R^{L13}). Provided that at least one of R^{a11} or R^{a12} represents an aromatic ring group represented by Formula (X).

Examples of the above-described aromatic ring group include an aryl group and a heteroaryl group.

The above-described aryl group is preferably a phenyl group or a naphthyl group.

In a case where the aryl group is a phenyl group, the phenyl group preferably has a substituent, and the substituent is independently preferably an alkyl group (preferably having 1 to 3 carbon atoms).

In a case where the aryl group is a phenyl group, the number of substituents contained in the phenyl group is preferably 1 to 5, and more preferably 2 or 3.

Examples of one aspect of the aryl group include a group represented by Formula (AS).

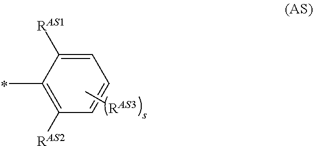

In the formulae, R^{AS1} and R^{AS2} represent an alkyl group (preferably having 1 to 3 carbon atoms). R^{AS3} represents a substituent. s represents an integer of 0 to 3. * represents a bonding position.

Examples of the substituent represented by R^{AS3} include the group exemplified by the above-described substituent W, and an alkyl group is preferable.

R^{L11} in-C(R^{L11})(R^{L12})(R^{L13}) represents a hydrogen atom, an alkyl group which may have a substituent, an aryl group which may have a substituent, or a heteroaryl group which may have a substituent. R^{L12} and R^{L13} each independently represent an alkyl group which may have a substituent, an aryl group which may have a substituent, or a heteroaryl group which may have a substituent.

R^{L11} to R^{L13} may be bonded to each other to form a ring.

Examples of an aspect in which R^{L11} to R^{L13} are bonded to each other to form a ring include the following aspects. For example, the alkyl groups which may have a substituent may be bonded to each other to form a ring. A substituent in the aryl group which may have a substituent and the alkyl group which may have a substituent may be bonded to each other to form a ring. A substituent in the heteroaryl group which may have a substituent and the alkyl group which may have a substituent may be bonded to each other to form a ring. A substituent in the aryl group which may have a substituent and a substituent in another aryl group which may have a substituent may be bonded to each other to form a ring. A substituent in the aryl group which may have a substituent and a substituent in the heteroaryl group which may have a substituent may be bonded to each other to form a ring. A substituent in the heteroaryl group which may have a substituent and a substituent in another heteroaryl group which may have a substituent may be bonded to each other to form a ring.

A substituent in the ring formed as described above, and another alkyl group which may have a substituent, a substituent in another aryl group which may have a substituent, or a substituent in another heteroaryl group which may have a substituent may be bonded to form a ring.

As described above, a group may be formed by bonding the substituent and the substituent (for example, the substituent in the aryl group which may have a substituent and the substituent in the heteroaryl group which may have a substituent) to form a single bond.

In a case where the alkyl group which may have a substituent, the aryl group which may have a substituent, and the heteroaryl group which may have a substituent, which are represented by R^{L11} to R^{L13}, may be bonded to each other to form a ring, —C(R^{L11})(R^{L12})(R^{L13}) is preferably a group other than the aryl group and the heteroaryl group.

The alkyl groups represented by R^{L11} to R^{L13} each independently may be any of linear, branched, or cyclic. In the alkyl groups represented by R^{L11} to R^{L13}, it is preferable that two alkyl groups are bonded to each other to form a ring.

More specifically, for example, the alkyl group represented by R^{L11} and the alkyl group represented by R^{L12} may be bonded to each other to form a ring. Furthermore, a substituent contained in a ring (a monocyclic cycloalkane ring or the like), which is formed by bonding the alkyl group represented by R^{L11} and the alkyl group represented by R^{L12} to each other, and an alkyl group represented by R¹¹³ may be bonded to each other to form a polycycle (a polycyclic cycloalkane ring or the like).

That is, —C(R^{L11})(R^{L12})(R^{L13}) may be a cycloalkyl group (preferably a cyclohexyl group) which may have a substituent. The number of membered rings of the above-described cycloalkyl group is preferably 3 to 12, more preferably 5 to 8, and still more preferably 6.

The cycloalkyl group may be monocyclic (a cyclohexyl group or the like) or polycyclic (1-adamantyl group or the like).

The cycloalkyl group preferably has a substituent. In a case where the cycloalkyl group has a substituent, a carbon atom adjacent to a carbon atom directly bonded to the nitrogen atom specified in General Formula (1) (that is, the “C” atom specified in “—C(R^{L11})(R^{L12})(R^{L13})”) preferably has a substituent.

An example of a substituent which may be contained in the cycloalkyl group includes an alkyl group (preferably having 1 to 3 carbon atoms).

Substituents contained in the cycloalkyl group may be bonded to each other to form a ring, and the ring formed by bonding the substituents to each other may be a ring other than a cycloalkane ring.

The aromatic ring group represented by Formula (X) is a group shown below.

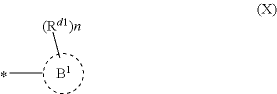

In Formula (X), B¹ represents an aromatic ring which may have a substituent other than R^{d1}.

Examples of the above-described aromatic ring represented by B¹ include a monocyclic or polycyclic aromatic hydrocarbon ring and a monocyclic or polycyclic aromatic heterocyclic ring.

Examples of the aromatic hydrocarbon ring and the aromatic heterocyclic ring are as described above.

From the viewpoint that the effect of the present invention is more excellent, among these, the aromatic ring represented by B¹ is preferably a monocyclic aromatic hydrocarbon ring (preferably 5- or 6-membered), more preferably a monocyclic aromatic heterocyclic ring (preferably 5- or 6-membered), and still more preferably a benzene ring.

Examples of the substituent other than R^{d1}, which may be included in B¹, include a group exemplified as the above-described substituent W, and among these, an alkyl group, a silyl group, an alkoxy group, an alkylthio group, a cyano group, a halogen atom, and the like are preferable.

R^{d1} represents an aryl group having a substituent A, a heteroaryl group having the substituent A, an alkenyl group having the substituent A, or an alkynyl group having the substituent A.

The substituent A represents an aryl group which may have a substituent or a heteroaryl group which may have a substituent.

The aryl group represented by the substituent A is preferably a monocyclic aryl group, and more preferably a phenyl group.

The heteroaryl group represented by the substituent A is preferably a monocyclic heteroaryl group. Examples of the aromatic heterocyclic ring constituting the heteroaryl group include a pyrrole ring, a furan ring, a thiophene ring, an imidazole ring, an oxazole ring, a pyridine ring, and the like.

The aryl group and the heteroaryl group, which are represented by the substituent A, may further have a substituent. Examples of the above-described substituent include the group exemplified by the above-described substituent W, and among these, examples thereof include an alkyl group, a silyl group, an alkoxy group, an alkylthio group, a cyano group, a halogen atom, an alkenyl group, an alkynyl group, a group represented by Formula (XA), and the like.

*—Ar—(R^{XA})ₚ Formula (XA):

In Formula (XA), Ar represents a (p+1)-valent aromatic ring group.

R^{XA} represents an alkyl group, a silyl group, an alkoxy group, an alkylthio group, a cyano group, a halogen atom, an alkenyl group, an alkynyl group, an aryl group, or a heteroaryl group.

p represents an integer of 0 to 5.

Examples of the aromatic ring constituting the (p+1)-valent aromatic ring group represented by Ar include an aromatic hydrocarbon ring and an aromatic heterocyclic ring.

The aromatic hydrocarbon ring is preferably a monocyclic aromatic hydrocarbon ring, and more preferably a benzene ring. The aromatic heterocyclic ring is preferably a monocyclic aromatic heterocyclic ring. Examples of the aromatic heterocyclic ring include a pyrrole ring, a furan ring, a thiophene ring, an imidazole ring, an oxazole ring, a pyridine ring, and the like.

The aryl group represented by R^{XA} is preferably a monocyclic aryl group and more preferably a phenyl group.

The heteroaryl group represented by R^{XA} is preferably a monocyclic heteroaryl group. Examples of the aromatic heterocyclic ring constituting the heteroaryl group include the same aromatic heterocyclic ring as the aromatic heterocyclic ring constituting the aromatic ring group represented by Ar.

Each group represented by R^{XA} may further have a substituent, as possible. For example, in a case where R^{XA} represents an aryl group or a heteroaryl group, examples of the substituent include a group exemplified as the above-described substituent, and among these, an alkyl group, a silyl group, an alkoxy group, an alkylthio group, a cyano group, a halogen atom, an alkenyl group, or an alkynyl group is preferable.

The substituent which may be contained in B¹ and R^{d1} may be bonded to each other to form a non-aromatic ring. Examples of an aspect in which the substituent which may be included in B¹ and R^{d1} are bonded to each other include an aspect in which the substituent which may be included in B¹ and a substituent which is included in the aryl group (or heteroaryl group) having the substituent A represented by R^{d1} in addition to the substituent A, are bonded to each other to form a non-aromatic ring (preferably a 5- to 6-membered ring), and the like.

Specific examples of the group represented by Formula (X) in a case where the substituent which may be included in B¹ and R^{d1} are bonded to each other to form a non-aromatic ring include the following groups, and the like.

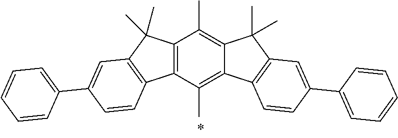 [SMILES: Cc1c2c(c(C)c3c1-c1ccc(-c4ccccc4)cc1C3(C)C)C(C)(C)c1cc(-c3ccccc3)ccc1-2]

In addition, R^{d1}'s may be bonded to each other to form a non-aromatic ring. Examples of the aspect in which R^{d1}'s are bonded to each other include an aspect in which a substituent which may be included in an aryl group and a heteroaryl group, which is represented by a substituent A in an aryl group (or a heteroaryl group) having a substituent A represented by one R^{d1} and a substituent which may be included in an aryl group and a heteroaryl group, which is represented by a substituent A in an aryl group (or a heteroaryl group) having a substituent A represented by the other R^{d1} are bonded to each other to form a non-aromatic ring (preferably a 5- to 6-membered ring).

In addition, a plurality of R^{d1}'s present in Formula (X) may be the same or different from each other.

In Formula (X), n represents an integer of 2 or 3, and is preferably 2.

In Formula (X), * represents a bonding position.

Among these, the aromatic ring group represented by Formula (X) is preferably the aromatic ring group represented by Formula (X′).

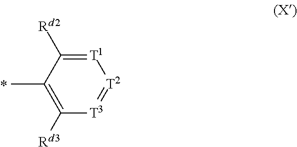

In Formula (X′), T¹ to T³ each independently represent —CR^{e12}═ or a nitrogen atom (=N—). R^{e12} represents a hydrogen atom or a substituent.

Examples of the substituent represented by R^{e12} include the group exemplified by the above-described substituent W, and among these, an alkyl group, an aryl group, a heteroaryl group, a silyl group, a halogen atom, or a cyano group is preferable.

R^{d2} and R^{d3} each independently have the same meaning as R^{d1} in Formula (X), and suitable aspects thereof are also the same.

In a case where T¹ represents —CR^{e12}═, R^{d2} and R^{e12} may be bonded to each other to form a non-aromatic ring. In a case where T³ represents —CR^{e12}═, R^{d3} and R^{e12} may be bonded to each other to form a non-aromatic ring.

Examples of an aspect in which R^{e12} and R^{d2} are bonded to each other include an aspect in which R^{e12} and a substituent which is included in the aryl group (or heteroaryl group) having the substituent A represented by R^{d2} in addition to the substituent A, are bonded to each other to form a non-aromatic ring (preferably a 5- to 6-membered ring), and the like.

In addition, examples of an aspect in which R^{e12} and R^{d3} are bonded to each other include an aspect in which R^{e12} and a substituent which is included in the aryl group (or heteroaryl group) having the substituent A represented by R^{d3} in addition to the substituent A, are bonded to each other to form a non-aromatic ring (preferably a 5- to 6-membered ring), and the like.

In addition, a plurality of R^{d1}'s present in Formula (X′) may be the same or different from each other.

* represents a bonding position.

Ar¹¹ represents an aromatic ring which includes at least two carbon atoms (intended to be two carbon atoms specified in Formula (1)) and may have a substituent.

Ar¹¹ is preferably an aromatic heterocyclic ring, and more preferably a quinoxaline ring or a pyrazine ring.

Examples of the substituent included in the aromatic ring represented by Ar¹¹ include the group exemplified by the above-described substituent W, and among these, an alkyl group, a halogen atom, or a cyano group is preferable, and an alkyl group is more preferable.

The group represented by Formula (1) is preferably a group represented by Formula (1A), more preferably a group represented by Formula (1B), and still more preferably a group represented by Formula (1C), from the viewpoint that the effect of the present invention is more excellent.

Hereinafter, each of Formulae (1A) to (1C) will be described.

### <Compound Represented by Formula (1A)>

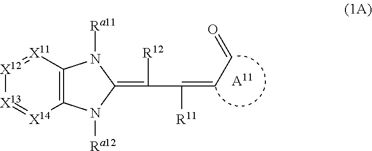

In Formula (1A), A¹¹, R¹¹, R¹², R^{a11}, and R^{a12} have the same meanings as A¹¹, R¹¹, R¹², R^{a11}, and R^{a12} in Formula (1), and suitable aspects thereof are also the same.

X¹¹ to X¹⁴ each independently represent a nitrogen atom or —CR^{c11}═. R^{c11} represents a hydrogen atom or a substituent. In a case where a plurality of R^{c11}'s are present, the plurality of R^{c11}'s may be bonded to each other to form a ring.

Examples of the substituent represented by R^{c11} include the group exemplified by the above-described substituent W.

It is preferable that at least two of X¹¹, . . . , or X¹⁴ are nitrogen atoms, it is more preferable that at least X¹¹ and X¹⁴ are nitrogen atoms, and it is still more preferable that only X¹¹ and X¹⁴ are nitrogen atoms.

The ring formed by bonding R^{c11}'s to each other is preferably an aromatic ring, and more preferably a benzene ring or a pyridine ring. The ring formed by bonding the R^{c11}'s to each other may further have a substituent (for example, the group exemplified by the above-described substituent W).

### <Compound Represented by Formula (1B)>

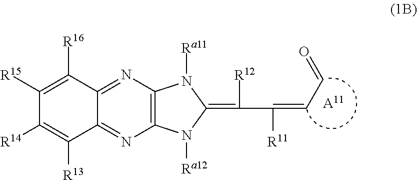

In Formula (1B), A¹¹, R¹¹, R¹², R^{a11}, and R^{a12} have the same meanings as A¹¹, R¹¹, R¹², R^{a11}, and R^{a12} in Formula (1), and suitable aspects thereof are also the same.

R¹³ to R¹⁶ each independently represent a hydrogen atom or a substituent.

Examples of the substituent represented by R¹³ to R¹⁶ include the groups exemplified by the above-described substituent W, and an alkyl group, a halogen atom, or a cyano group is preferable and an alkyl group, a fluorine atom, a chlorine atom, or a cyano group is more preferable.

In addition, R¹³ and R¹⁴, R¹⁴ and R¹⁵, and R¹⁵ and R¹⁶ each may be independently bonded to each other to form a ring. The ring formed by linking R¹³ and R¹⁴, R¹⁴ and R¹⁵, and R¹⁵ and R¹⁶ to each other is preferably a benzene ring or a pyridine ring. In addition, the ring formed by linking R¹³ and R¹⁴, R¹⁴ and R¹⁵, and R¹⁵ and R¹⁶ to each other may further have a substituent (for example, the group exemplified by the above-described substituent W).

### <Compound Represented by Formula (1C)>

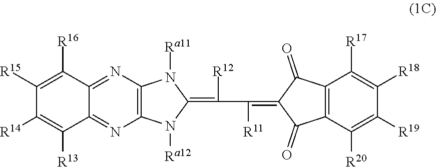

In Formula (1C), R¹¹, R¹², R^{a11}, and R^{a12} have the same meanings as R¹¹, R¹², R^{a11}, and R^{a12} in Formula (1), and suitable aspects thereof are also the same.

In Formula (1C), R¹³ to R¹⁶ have the same meaning as R¹³ to R¹⁶ in Formula (1B), and suitable aspects thereof are also the same.

In Formula (1C), R¹⁷ to R²⁰ each independently represent a hydrogen atom or a substituent.

Examples of the substituent represented by R¹⁷ to R²⁰ include the group exemplified by the above-described substituent W, and a halogen atom is preferable and a fluorine atom or a chlorine atom is more preferable.

R¹⁷ to R²⁰ are preferably a hydrogen atom or a chlorine atom, and more preferably a hydrogen atom.

R¹⁷ and R¹⁸, R¹⁸ and R¹⁹, and R¹⁹ and R²⁰ each may be independently bonded to each other to form a ring. The ring formed by linking R¹⁷ and R¹⁸, R¹⁸ and R¹⁹, and R¹⁹ and R²⁰ to each other is preferably a benzene ring. Among these, R¹⁸ and R¹⁹ are preferably bonded to each other to form a ring, and the ring formed by bonding R¹⁸ and R¹⁹ to each other is preferably a benzene ring. The ring formed by linking R 18 and R¹⁹ to each other may be further substituted with a substituent (for example, the group exemplified by the above-described substituent W).

### <Compound Represented by Formula (2)>

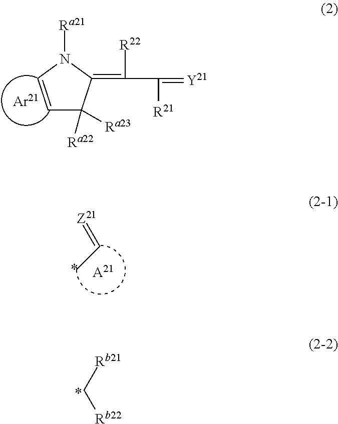

In Formula (2), Y²¹ represents a group represented by Formula (2-1) or a group represented by Formula (2-2).

*, A²¹, and Z²¹ in Formula (2-1) each have the same meaning as *, A¹¹, and Z¹¹ in Formula (1-1), and suitable aspects thereof are also the same.

*, R^{b21}, and R^{b22} in Formula (2-2) each have the same meanings as *, R^{b11}, and R^{b12} in Formula (1-2), and suitable aspects thereof are also the same.

In Formula (2), R²¹ and R²² have the same meaning as R¹¹ and R¹² in Formula (1), and suitable aspects thereof are also the same.

Ar²¹ represents an aromatic ring which may have at least two carbon atoms (intended to be two carbon atoms specified in Formula (2)) and a substituent.

Ar²¹ is preferably an aromatic hydrocarbon group, more preferably a benzene ring or a naphthalene ring, and still more preferably a benzene ring.

Examples of the substituent included in the aromatic ring represented by Ar²¹ include the group exemplified by the above-described substituent W, and an alkyl group, a halogen atom, or a cyano group is preferable, and an alkyl group or a chlorine atom is more preferable.

R^{a21} to R^{a23} each independently represent an alkyl group which may have a substituent or an aromatic ring group which may have a substituent.

Examples of the aromatic ring group represented by R^{a21} to R^{a23}, which may have a substituent, include the same aromatic ring group as the aromatic ring group represented by R^{a11} and R^{a12}, which may have a substituent.

Provided that at least one of R^{a21}, R^{a22}, or R^{a23} represents an aromatic ring group represented by Formula (X). The aromatic ring group represented by Formula (X) is the same group as the aromatic ring group represented by Formula (X) described as the group which is included in at least one of R^{a1} or R^{a2} in Formula (1), and a suitable aspect is also the same.

In addition, R^{a22} and R^{a23} may be bonded to each other to form a ring. The ring formed by the bonding of R^{a22} and R^{a23} to each other is preferably a non-aromatic ring, and more preferably a cycloalkane ring. The number of membered rings of the cycloalkane ring is preferably 3 to 12, more preferably 5 to 8, and still more preferably 6.

The cycloalkane ring may be a monocycle (cyclohexyl group or the like) or a polycyclic (1-adamantyl group or the like).

The cycloalkane ring may have a substituent. Examples of the substituent include the group exemplified by the above-described substituent W, and the like.

### <Compound Represented by Formula (3)>

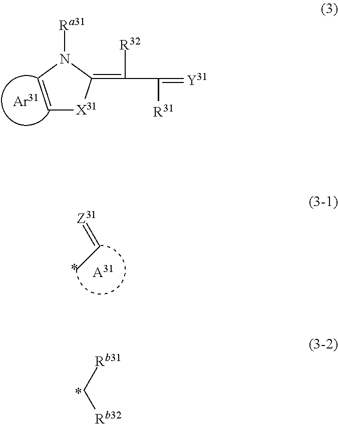

In Formula (3), Y³¹ represents a group represented by Formula (3-1) or a group represented by Formula (3-2).

*, A³¹, and Z³¹ in Formula (3-1) each have the same meaning as *, A¹¹, and Z¹¹ in Formula (1-1), and suitable aspects thereof are also the same.

*, R^{b31}, and R^{b32} in Formula (3-2) each have the same meanings as *, R^{b11}, and R^{b12} in Formula (1-2), and suitable aspects thereof are also the same.

In Formula (3), R³¹ and R³² have the same meaning as R¹¹ and R¹² in Formula (1), and suitable aspects thereof are also the same.

Ar³¹ represents an aromatic ring which includes at least two carbon atoms (intended to be two carbon atoms specified in Formula (3)) and may have a substituent.

Ar³¹ is preferably an aromatic hydrocarbon group, more preferably a benzene ring or a naphthalene ring, and still more preferably a benzene ring.

Examples of the substituent included in the aromatic ring represented by Ar³¹ include the group exemplified by the above-described substituent W, and an alkyl group, a halogen atom, or a cyano group is preferable, and an alkyl group or a chlorine atom is more preferable.

X³¹ represents an oxygen atom or a sulfur atom.

R^{a31} represents an aromatic ring group represented by Formula (X). The aromatic ring group represented by Formula (X) is the same group as the aromatic ring group represented by Formula (X) described as the group which is included in at least one of R^{a1} or R^{a2} in Formula (1), and a suitable aspect is also the same.

### <Compound Represented by Formula (4)>

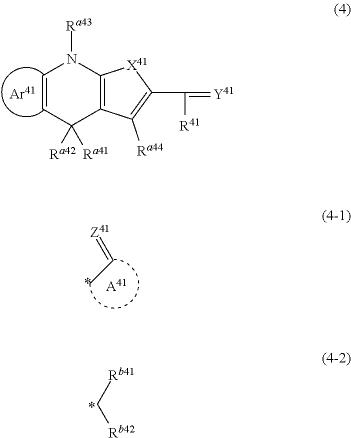

In Formula (4), Y⁴¹ represents a group represented by Formula (4-1) or a group represented by Formula (4-2).

*, A⁴¹, and Z⁴¹ in Formula (4-1) each have the same meaning as *, A¹¹, and Z¹¹ in Formula (1-1), and suitable aspects thereof are also the same.

*, R^{b41}, and R^{b42} in Formula (4-2) each have the same meanings as *, R^{b11}, and R^{b12} in Formula (1-2), and suitable aspects thereof are also the same.

In Formula (4), R⁴¹ has the same meaning as R¹¹ in Formula (1), and a suitable aspect thereof is also the same.

In Formula (4), Ar⁴¹ represents an aromatic ring which includes at least two carbon atoms (intended to be two carbon atoms specified in Formula (4)) and may have a substituent.

Ar⁴¹ is preferably an aromatic hydrocarbon group, more preferably a benzene ring or a naphthalene ring, and still more preferably a benzene ring.

Examples of the substituent included in the aromatic ring represented by Ar⁴¹ include the group exemplified by the above-described substituent W, and an alkyl group, a halogen atom, or a cyano group is preferable, and an alkyl group or a chlorine atom is more preferable.

X⁴¹ represents an oxygen atom, a sulfur atom, —NR^{CT1}—, —CR^{CT2}R^{CT3}—, or —C(R^{CT4})═C(R^{CT5})—.

X⁴¹ is preferably a sulfur atom, —NR^{CT1}—, or —C(R^{CT4})—C(R^{CT5})—.

R^{CT1} to R^{CT5} each independently represent a hydrogen atom or a substituent.

Examples of the substituent represented by R^{CT1} to R^{CT5} include the group exemplified by the above-described substituent W.

Among those, R^{CT1} to R^{CT5} is preferably a hydrogen atom.

R^{a41} to R^{a44} each independently represent a hydrogen atom, an alkyl group which may have a substituent, or an aromatic ring group which may have a substituent.

Examples of the aromatic ring group represented by R^{a41} to R^{a44}, which may have a substituent, include the same aromatic ring group as the aromatic ring group represented by R^{a11} and R^{a12}, which may have a substituent.

Among these, it is preferable that R^{a44} represents a hydrogen atom, and R^{a41} to R^{a43} represent a group other than a hydrogen atom.

Provided that at least one of R^{a41} or R^{a44} represents an aromatic ring group represented by Formula (X). The aromatic ring group represented by Formula (X) is the same group as the aromatic ring group represented by Formula (X) described as the group which is included in at least one of R^{a1} or R^{a2} in Formula (1), and a suitable aspect is also the same.

In addition, R^{a41} and R^{a42} may be bonded to each other to form a ring. The ring formed by the bonding of R^{a41} and R^{a42} to each other is preferably a non-aromatic ring, and more preferably a cycloalkane ring. The number of membered rings of the cycloalkane ring is preferably 3 to 12, more preferably 5 to 8, and still more preferably 6.

The cycloalkane ring may be a monocycle (cyclohexyl group or the like) or a polycyclic (1-adamantyl group or the like).

The cycloalkane ring may have a substituent. Examples of the substituent include the group exemplified by the above-described substituent W, and the like.

### <Compound Represented by Formula (5)>

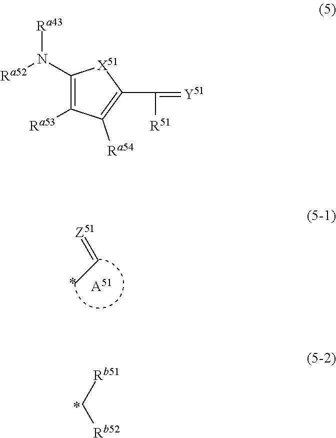

In Formula (5), Y⁵¹ represents a group represented by Formula (5-1) or a group represented by Formula (5-2).

*, A⁵¹, and Z⁵¹ in Formula (5-1) each have the same meaning as *, A¹¹, and Z¹¹ in Formula (1-1), and suitable aspects thereof are also the same.

*, R^{b51}, and R^{b52} in Formula (5-2) each have the same meanings as *, R^{b11}, and R^{b12} in Formula (1-2), and suitable aspects thereof are also the same.

In Formula (5), R⁵¹ has the same meaning as R¹¹ in Formula (1), and a suitable aspect thereof is also the same.

X⁵¹ has the same meaning as X⁴¹ in Formula (4), and a suitable aspect thereof is also the same.

R^{a51} and R^{a52} each independently represent an alkyl group which may have a substituent or an aromatic ring group which may have a substituent.

R^{a53} and R^{a54} each independently represent a hydrogen atom, an alkyl group which may have a substituent, or an aromatic ring group which may have a substituent.

Examples of the aromatic ring group represented by R^{a51} to R^{a54}, which may have a substituent, include the same aromatic ring group as the aromatic ring group represented by R^{a11} and R^{a12}, which may have a substituent.

Provided that at least one of R^{a51}, . . . , or R^{a54} represents an aromatic ring group represented by Formula (X). The aromatic ring group represented by Formula (X) is the same group as the aromatic ring group represented by Formula (X) described as the group which is included in at least one of R^{a1} or R^{a2} in Formula (1), and a suitable aspect is also the same.

### <Compound Represented by Formula (6)>

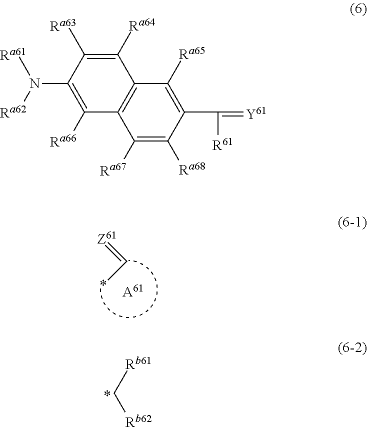

In Formula (6), Y⁶¹ represents a group represented by Formula (6-1) or a group represented by Formula (6-2).

*, A⁶¹, and Z⁶¹ in Formula (6-1) each have the same meaning as *, A¹¹, and Z¹¹ in Formula (1-1), and suitable aspects thereof are also the same.

*, R^{b61}, and R^{b62} in Formula (6-2) each have the same meanings as *, R^{b11}, and R^{b12} in Formula (1-2), and suitable aspects thereof are also the same.

In Formula (6), R⁶¹ has the same meaning as R¹¹ in Formula (1), and a suitable aspect thereof is also the same.

R^{a61} and R^{a62} each independently represent an alkyl group which may have a substituent or an aromatic ring group which may have a substituent.

R^{a63} to R^{a68} each independently represent a hydrogen atom, an alkyl group which may have a substituent, or an aromatic ring group which may have a substituent.

Examples of the aromatic ring group represented by R^{a61} to R^{a68}, which may have a substituent, include the same aromatic ring group as the aromatic ring group represented by R^{a11} and R^{a12}, which may have a substituent.

Provided that at least one of R^{a61}, . . . , or R^{a68} represents an aromatic ring group represented by Formula (X). The aromatic ring group represented by Formula (X) is the same group as the aromatic ring group represented by Formula (X) described as the group which is included in at least one of R^{a1} or R^{a2} in Formula (1), and a suitable aspect is also the same.

Hereinafter, specific examples of the specific compound will be described, but the present embodiment is not limited thereto.

Specific examples of the specific compound include the compound represented by Formula (1D), compound described later, and the like.

In the specific compounds exemplified below, both the cis isomer and the trans isomer which are distinguished based on the C═C double bond in the compound are each included in the specific compounds exemplified below.

### (Compound Represented by Formula (1D))

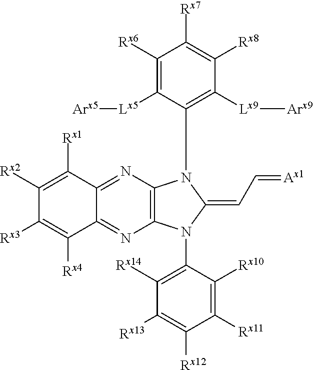

In the compound represented by Formula (1D), L^{x5} and L^{x9} represent a divalent linking group selected from the following group of the linking groups L1. In the following group of the linking groups L1, * represents a bonding position. “Me” and “t-Bu” each represent a methyl group and a t-butyl group.

### (Group of Linking Groups L1)

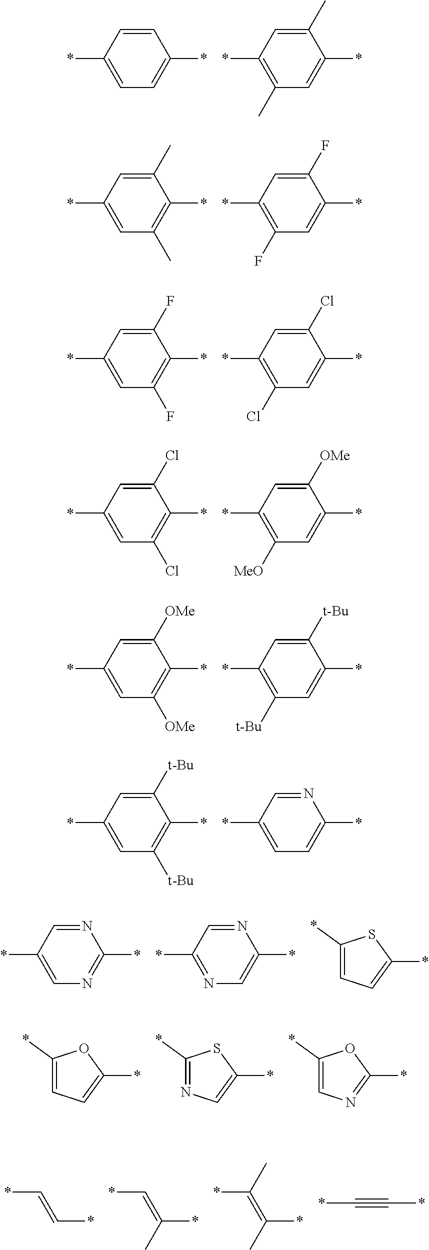

In the compound represented by Formula (1D), Ar^{x5} and Ar^{x9} represent a substituent selected from the following substituent group A¹. In the following substituent group A¹, * represents a bonding position. “Me” represents a methyl group.

### (Substituent Group A¹)

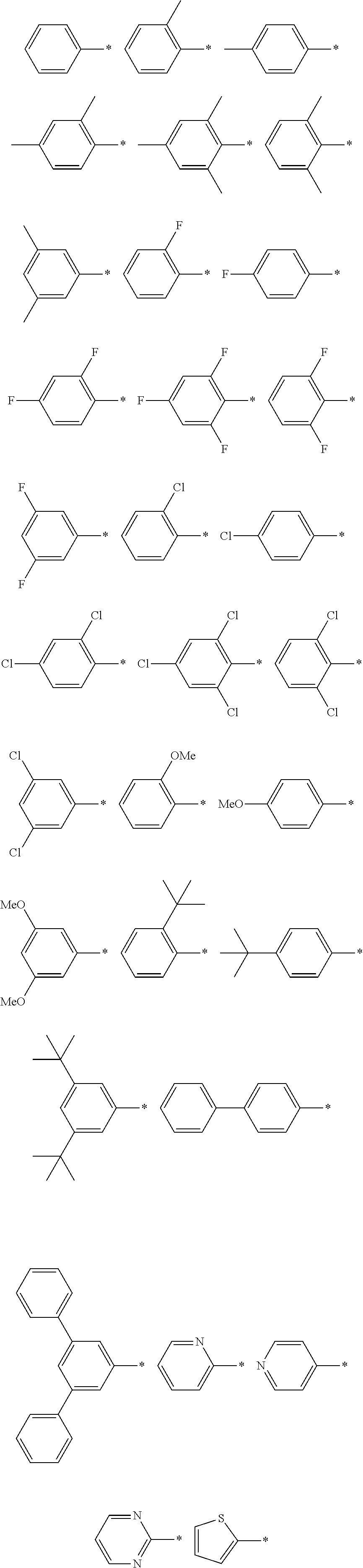

In the compound represented by Formula (1D), examples of a combination of R^{x1} to R^{x4} include a combination selected from the following substituent group A², examples of a combination of R^{x6} to R^{x8} include a combination selected from the following substituent group A3, and examples of a combination of R^{x10} to R^{x14} include a combination selected from the following substituent group A4. In the following substituent groups A2 to A4, “Me”, “i-Pr”, and “t-Bu” each represent a methyl group, an isopropyl group, and a t-butyl group.

### (Substituent Group A2)

TABLE 1

| No. | Rx¹ | Rx² | Rx³ | Rx⁴ |
|---|---|---|---|---|
| 1 | H | H | H | H |
| 2 | H | Cl | Cl | H |
| 3 | H | F | F | H |
| 4 | H | Me | Me | H |

### (Substituent Group A3)

TABLE 2

| No. | Rx⁶ | Rx⁷ | Rx⁸ |
|---|---|---|---|
| 1 | H | H | H |
| 2 | H | Br | H |
| 3 | H | Cl | H |
| 4 | H | Me | H |
| 5 | H | t-Bu | H |
| 6 | Me | Me | Me |

### (Substituent Group A4)

TABLE 3

| No. | Rx¹⁰ | Rx¹¹ | Rx¹² | Rx¹³ | Rx¹⁴ |
|---|---|---|---|---|---|
| 1 | Et | H | H | H | Et |
| 2 | Et | H | Me | H | Et |
| 3 | i-Pr | H | H | H | i-Pr |
| 4 | Me | H | Br | H | Me |
| 5 | Me | H | Cl | H | Me |
| 6 | Me | H | H | H | Me |
| 7 | Me | H | H | H | t-Bu |
| 8 | Me | H | Me | H | Me |
| 9 | Me | Me | Me | Me | Me |
| 10 | t-Bu | H | t-Bu | H | t-Bu |

In the compound represented by Formula (1D), A^{x1} represents a substituent selected from the following substituent group B1. In the following substituent group B1, * represents a bonding position.

### (Substituent Group B1)

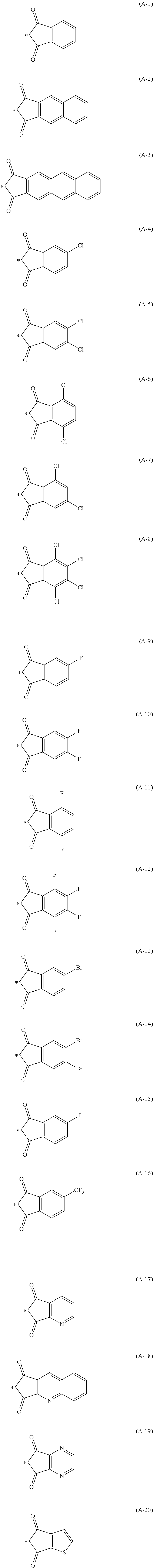

Examples of the specific compound also include the following compounds, in addition to the above-described compounds.

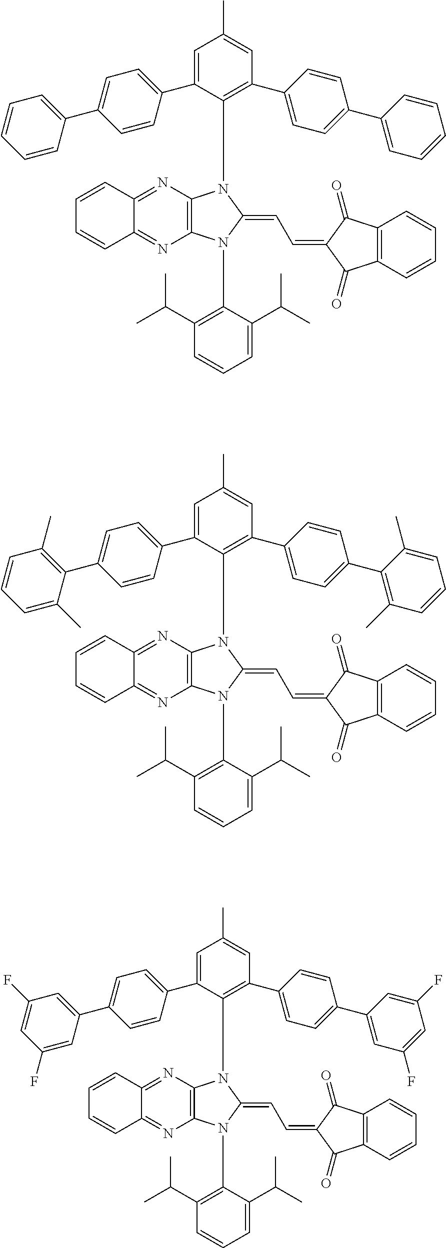 [SMILES: Cc1cc(-c2ccc(-c3ccccc3)cc2)c(N2/C(=C/C=C3C(=O)c4ccccc4C3=O)N(c3c(C(C)C)cccc3C(C)C)c3nc4ccccc4nc32)c(-c2ccc(-c3ccccc3)cc2)c1 ; Cc1cc(-c2ccc(-c3c(C)cccc3C)cc2)c(N2/C(=C/C=C3C(=O)c4ccccc4C3=O)N(c3c(C(C)C)cccc3C(C)C)c3nc4ccccc4nc32)c(-c2ccc(-c3c(C)cccc3C)cc2)c1 ; Cc1cc(-c2ccc(-c3cc(F)cc(F)c3)cc2)c(N2/C(=C/C=C3C(=O)c4ccccc4C3=O)N(c3c(C(C)C)cccc3C(C)C)c3nc4ccccc4nc32)c(-c2ccc(-c3cc(F)cc(F)c3)cc2)c1]

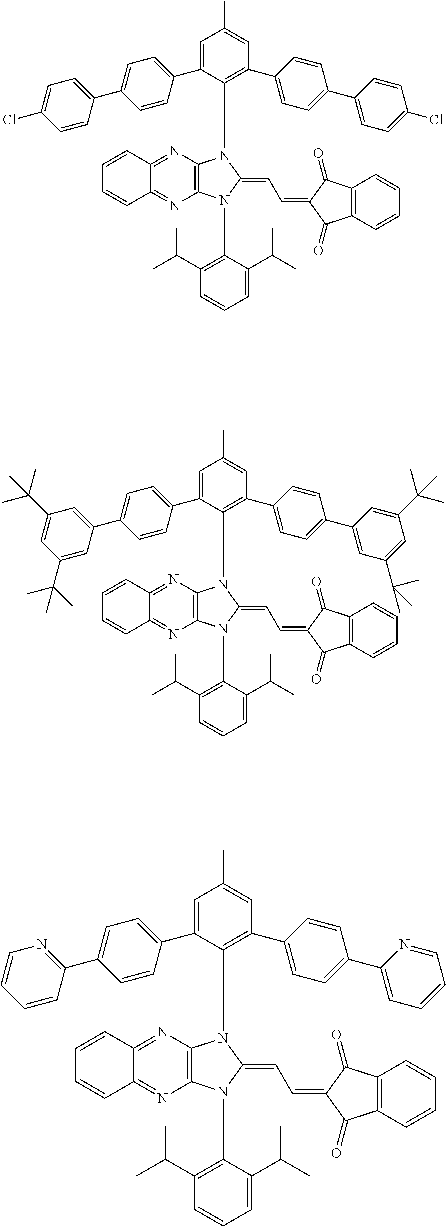 [SMILES: Cc1cc(-c2ccc(-c3ccc(Cl)cc3)cc2)c(N2/C(=C/C=C3C(=O)c4ccccc4C3=O)N(c3c(C(C)C)cccc3C(C)C)c3nc4ccccc4nc32)c(-c2ccc(-c3ccc(Cl)cc3)cc2)c1 ; Cc1cc(-c2ccc(-c3cc(C(C)(C)C)cc(C(C)(C)C)c3)cc2)c(N2/C(=C/C=C3C(=O)c4ccccc4C3=O)N(c3c(C(C)C)cccc3C(C)C)c3nc4ccccc4nc32)c(-c2ccc(-c3cc(C(C)(C)C)cc(C(C)(C)C)c3)cc2)c1 ; Cc1cc(-c2ccc(-c3ccccn3)cc2)c(N2/C(=C/C=C3C(=O)c4ccccc4C3=O)N(c3c(C(C)C)cccc3C(C)C)c3nc4ccccc4nc32)c(-c2ccc(-c3ccccn3)cc2)c1]

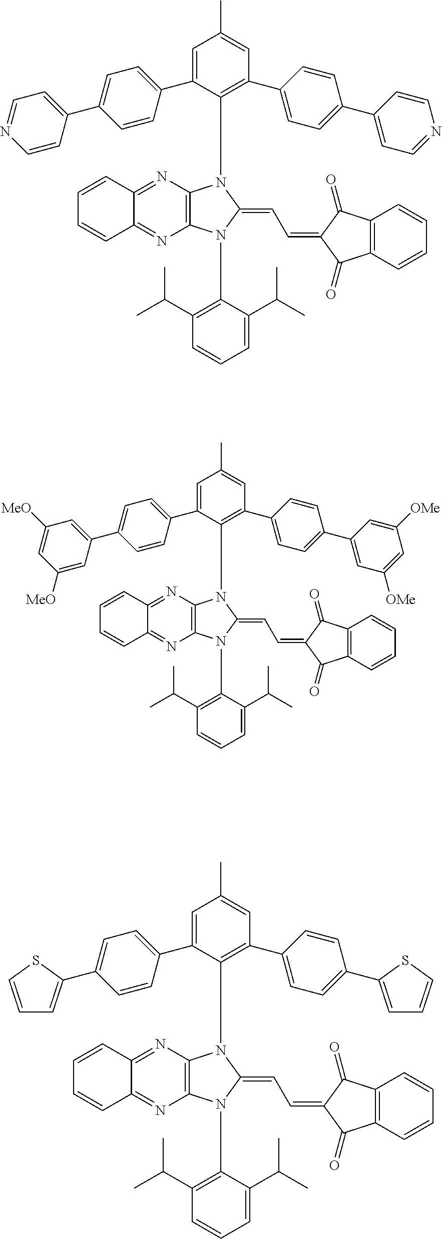 [SMILES: Cc1cc(-c2ccc(-c3ccncc3)cc2)c(N2/C(=C/C=C3C(=O)c4ccccc4C3=O)N(c3c(C(C)C)cccc3C(C)C)c3nc4ccccc4nc32)c(-c2ccc(-c3ccncc3)cc2)c1 ; COc1cc(OC)cc(-c2ccc(-c3cc(C)cc(-c4ccc(-c5cc(OC)cc(OC)c5)cc4)c3N3/C(=C/C=C4C(=O)c5ccccc5C4=O)N(c4c(C(C)C)cccc4C(C)C)c4nc5ccccc5nc43)cc2)c1 ; Cc1cc(-c2ccc(-c3cccs3)cc2)c(N2/C(=C/C=C3C(=O)c4ccccc4C3=O)N(c3c(C(C)C)cccc3C(C)C)c3nc4ccccc4nc32)c(-c2ccc(-c3cccs3)cc2)c1]

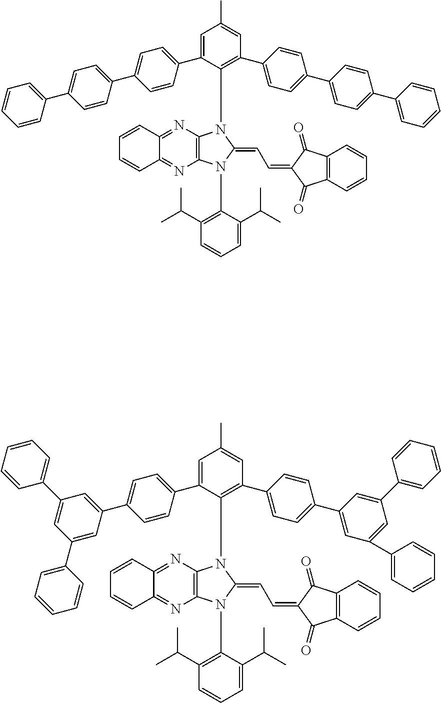 [SMILES: Cc1cc(-c2ccc(-c3ccc(-c4ccccc4)cc3)cc2)c(N2/C(=C/C=C3C(=O)c4ccccc4C3=O)N(c3c(C(C)C)cccc3C(C)C)c3nc4ccccc4nc32)c(-c2ccc(-c3ccc(-c4ccccc4)cc3)cc2)c1 ; Cc1cc(-c2ccc(-c3cc(-c4ccccc4)cc(-c4ccccc4)c3)cc2)c(N2/C(=C/C=C3C(=O)c4ccccc4C3=O)N(c3c(C(C)C)cccc3C(C)C)c3nc4ccccc4nc32)c(-c2ccc(-c3cc(-c4ccccc4)cc(-c4ccccc4)c3)cc2)c1]

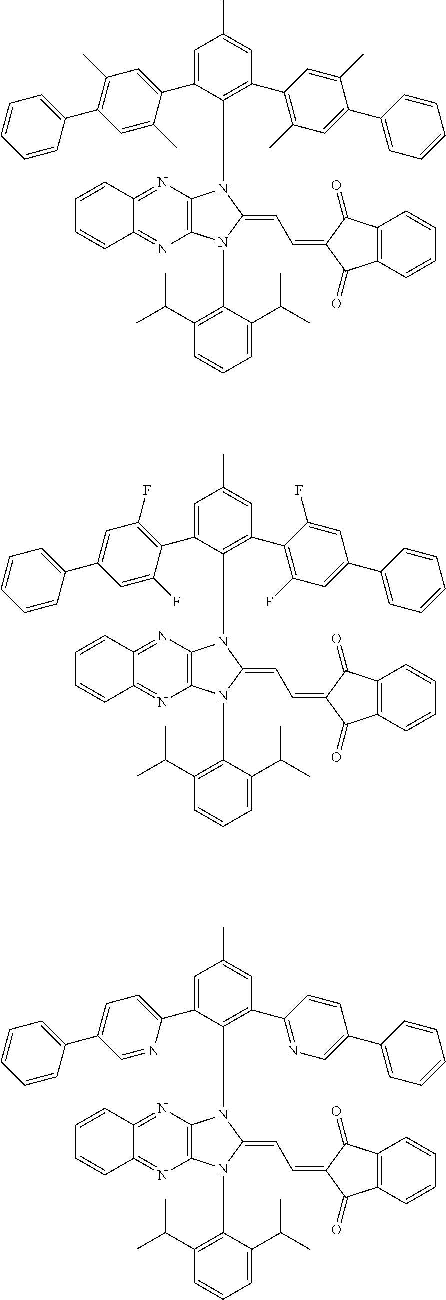 [SMILES: Cc1cc(-c2cc(C)c(-c3ccccc3)cc2C)c(N2/C(=C/C=C3C(=O)c4ccccc4C3=O)N(c3c(C(C)C)cccc3C(C)C)c3nc4ccccc4nc32)c(-c2cc(C)c(-c3ccccc3)cc2C)c1 ; Cc1cc(-c2c(F)cc(-c3ccccc3)cc2F)c(N2/C(=C/C=C3C(=O)c4ccccc4C3=O)N(c3c(C(C)C)cccc3C(C)C)c3nc4ccccc4nc32)c(-c2c(F)cc(-c3ccccc3)cc2F)c1 ; Cc1cc(-c2ccc(-c3ccccc3)cn2)c(N2/C(=C/C=C3C(=O)c4ccccc4C3=O)N(c3c(C(C)C)cccc3C(C)C)c3nc4ccccc4nc32)c(-c2ccc(-c3ccccc3)cn2)c1]

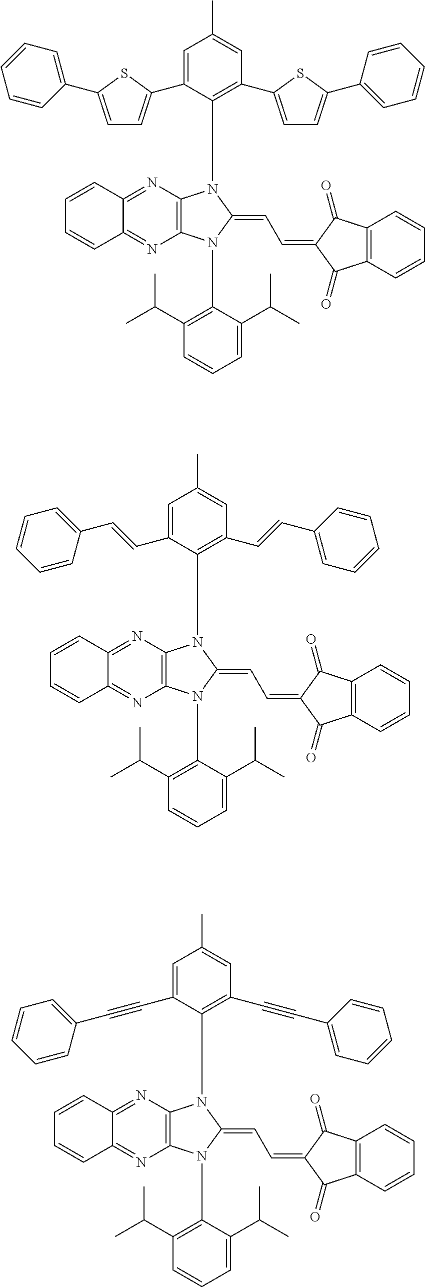 [SMILES: Cc1cc(-c2ccc(-c3ccccc3)s2)c(N2/C(=C/C=C3C(=O)c4ccccc4C3=O)N(c3c(C(C)C)cccc3C(C)C)c3nc4ccccc4nc32)c(-c2ccc(-c3ccccc3)s2)c1 ; Cc1cc(/C=C/c2ccccc2)c(N2/C(=C/C=C3C(=O)c4ccccc4C3=O)N(c3c(C(C)C)cccc3C(C)C)c3nc4ccccc4nc32)c(/C=C/c2ccccc2)c1 ; Cc1cc(C#Cc2ccccc2)c(N2/C(=C/C=C3C(=O)c4ccccc4C3=O)N(c3c(C(C)C)cccc3C(C)C)c3nc4ccccc4nc32)c(C#Cc2ccccc2)c1]

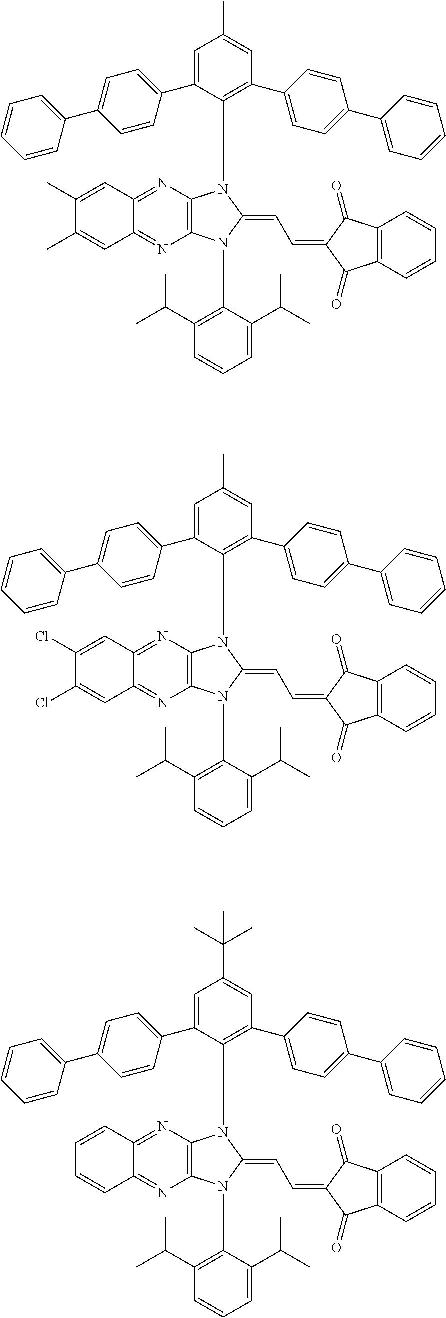 [SMILES: Cc1cc(-c2ccc(-c3ccccc3)cc2)c(N2/C(=C/C=C3C(=O)c4ccccc4C3=O)N(c3c(C(C)C)cccc3C(C)C)c3nc4cc(C)c(C)cc4nc32)c(-c2ccc(-c3ccccc3)cc2)c1 ; Cc1cc(-c2ccc(-c3ccccc3)cc2)c(N2/C(=C/C=C3C(=O)c4ccccc4C3=O)N(c3c(C(C)C)cccc3C(C)C)c3nc4cc(Cl)c(Cl)cc4nc32)c(-c2ccc(-c3ccccc3)cc2)c1 ; CC(C)c1cccc(C(C)C)c1N1/C(=C\C=C2C(=O)c3ccccc3C2=O)N(c2c(-c3ccc(-c4ccccc4)cc3)cc(C(C)(C)C)cc2-c2ccc(-c3ccccc3)cc2)c2nc3ccccc3nc21]

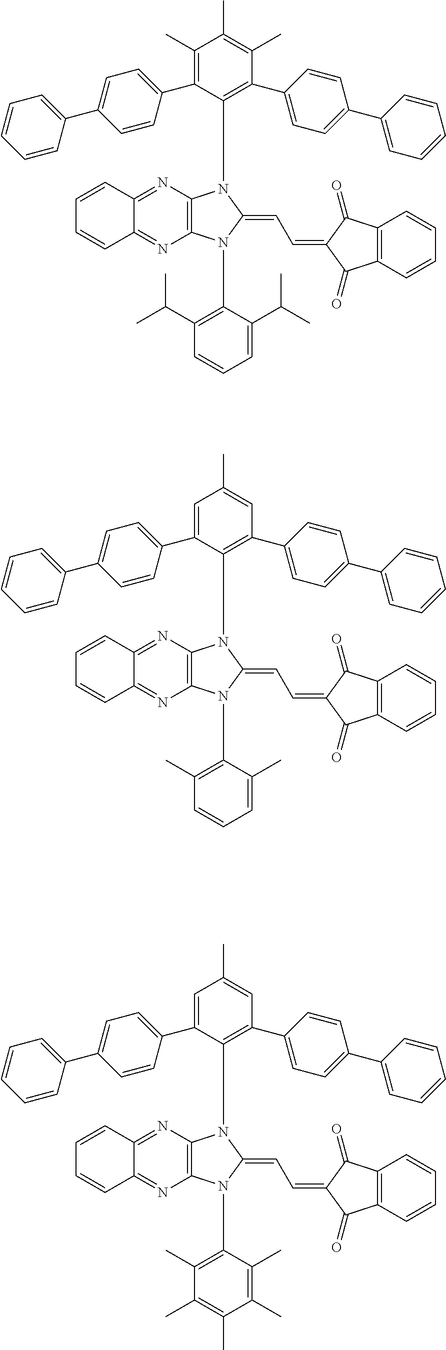 [SMILES: Cc1c(C)c(-c2ccc(-c3ccccc3)cc2)c(N2/C(=C/C=C3C(=O)c4ccccc4C3=O)N(c3c(C(C)C)cccc3C(C)C)c3nc4ccccc4nc32)c(-c2ccc(-c3ccccc3)cc2)c1C ; Cc1cc(-c2ccc(-c3ccccc3)cc2)c(N2/C(=C/C=C3C(=O)c4ccccc4C3=O)N(c3c(C)cccc3C)c3nc4ccccc4nc32)c(-c2ccc(-c3ccccc3)cc2)c1 ; Cc1cc(-c2ccc(-c3ccccc3)cc2)c(N2/C(=C/C=C3C(=O)c4ccccc4C3=O)N(c3c(C)c(C)c(C)c(C)c3C)c3nc4ccccc4nc32)c(-c2ccc(-c3ccccc3)cc2)c1]

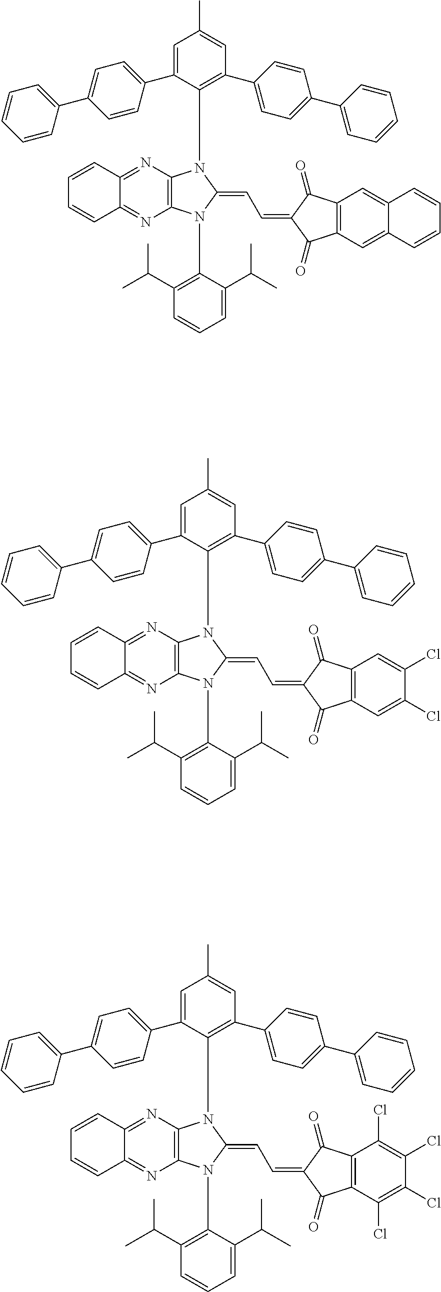 [SMILES: Cc1cc(-c2ccc(-c3ccccc3)cc2)c(N2/C(=C/C=C3C(=O)c4cc5ccccc5cc4C3=O)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C3C(=O)c4cc(Cl)c(Cl)cc4C3=O)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C3C(=O)c4c(Cl)c(Cl)c(Cl)c(Cl)c4C3=O)N(c3c(C(C)C)cccc3C(C)C)c3nc4ccccc4nc32)c(-c2ccc(-c3ccccc3)cc2)c1]

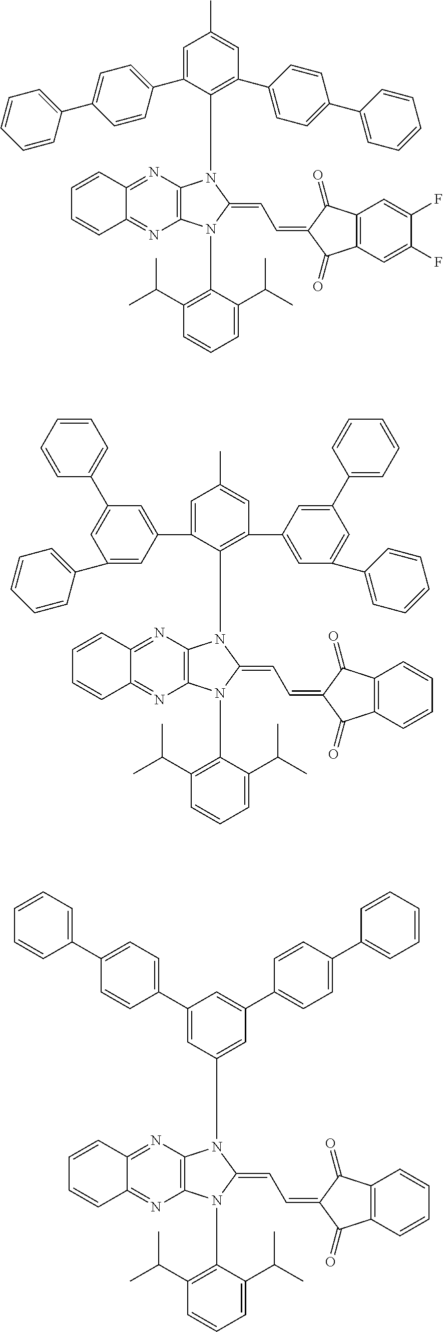 [SMILES: Cc1cc(-c2ccc(-c3ccccc3)cc2)c(N2/C(=C/C=C3C(=O)c4cc(F)c(F)cc4C3=O)N(c3c(C(C)C)cccc3C(C)C)c3nc4ccccc4nc32)c(-c2ccc(-c3ccccc3)cc2)c1 ; Cc1cc(-c2cc(-c3ccccc3)cc(-c3ccccc3)c2)c(N2/C(=C/C=C3C(=O)c4ccccc4C3=O)N(c3c(C(C)C)cccc3C(C)C)c3nc4ccccc4nc32)c(-c2cc(-c3ccccc3)cc(-c3ccccc3)c2)c1 ; CC(C)c1cccc(C(C)C)c1N1/C(=C\C=C2C(=O)c3ccccc3C2=O)N(c2cc(-c3ccc(-c4ccccc4)cc3)cc(-c3ccc(-c4ccccc4)cc3)c2)c2nc3ccccc3nc21]

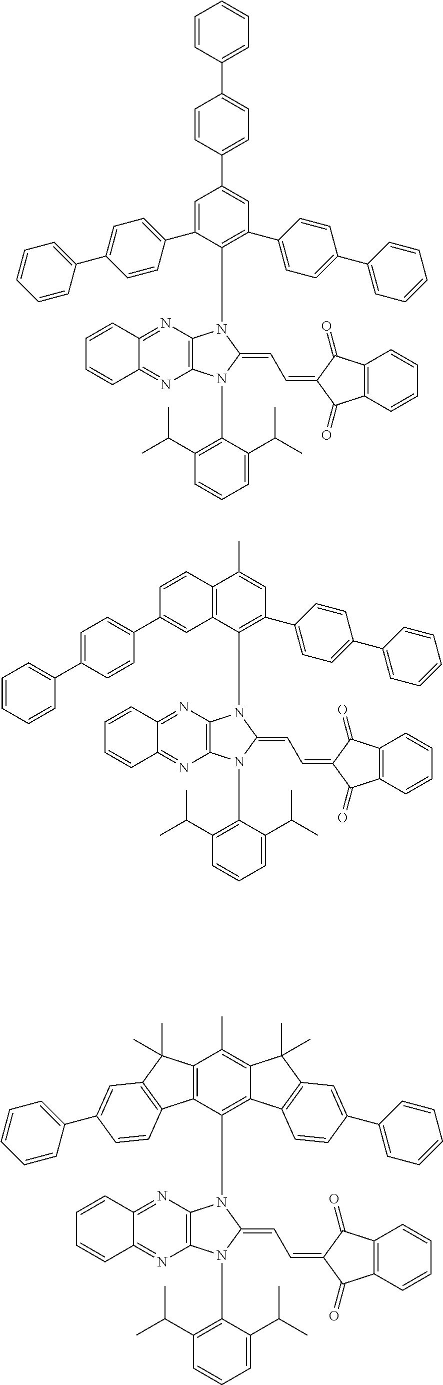 [SMILES: CC(C)c1cccc(C(C)C)c1N1/C(=C\C=C2C(=O)c3ccccc3C2=O)N(c2c(-c3ccc(-c4ccccc4)cc3)cc(-c3ccc(-c4ccccc4)cc3)cc2-c2ccc(-c3ccccc3)cc2)c2nc3ccccc3nc21 ; Cc1cc(-c2ccc(-c3ccccc3)cc2)c(N2/C(=C/C=C3C(=O)c4ccccc4C3=O)N(c3c(C(C)C)cccc3C(C)C)c3nc4ccccc4nc32)c2cc(-c3ccc(-c4ccccc4)cc3)ccc12 ; Cc1c2c(c(N3/C(=C/C=C4C(=O)c5ccccc5C4=O)N(c4c(C(C)C)cccc4C(C)C)c4nc5ccccc5nc43)c3c1C(C)(C)c1cc(-c4ccccc4)ccc1-3)-c1ccc(-c3ccccc3)cc1C2(C)C]

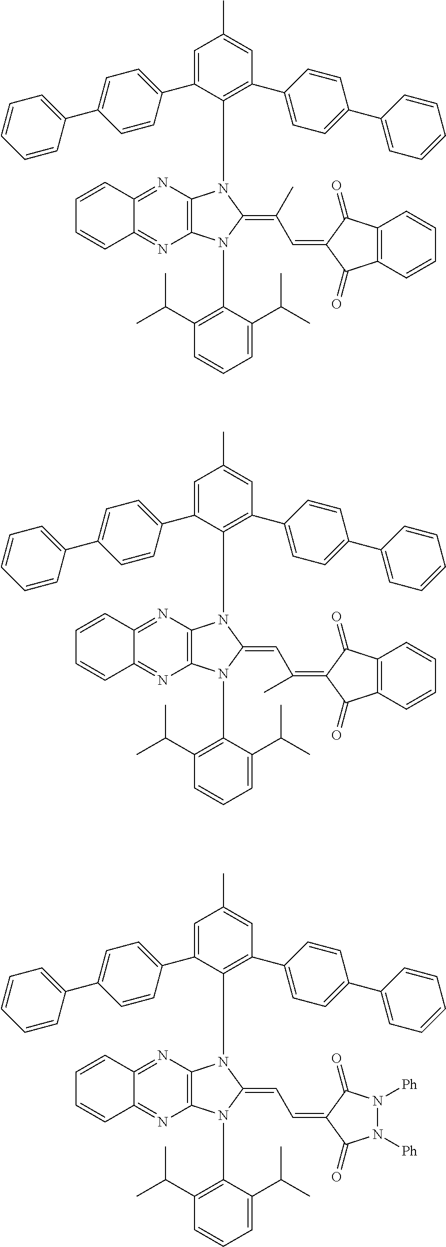 [SMILES: C/C(C=C1C(=O)c2ccccc2C1=O)=C1\N(c2c(-c3ccc(-c4ccccc4)cc3)cc(C)cc2-c2ccc(-c3ccccc3)cc2)c2nc3ccccc3nc2N1c1c(C(C)C)cccc1C(C)C ; CC(/C=C1/N(c2c(-c3ccc(-c4ccccc4)cc3)cc(C)cc2-c2ccc(-c3ccccc3)cc2)c2nc3ccccc3nc2N1c1c(C(C)C)cccc1C(C)C)=C1C(=O)c2ccccc2C1=O ; Cc1cc(-c2ccc(-c3ccccc3)cc2)c(N2/C(=C/C=C3C(=O)N(c4ccccc4)N(c4ccccc4)C3=O)N(c3c(C(C)C)cccc3C(C)C)c3nc4ccccc4nc32)c(-c2ccc(-c3ccccc3)cc2)c1]

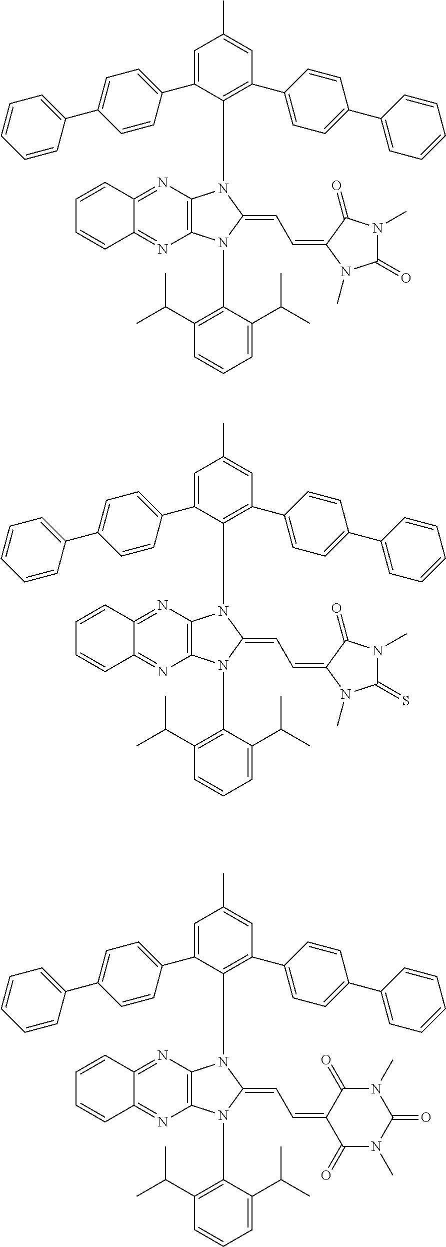 [SMILES: Cc1cc(-c2ccc(-c3ccccc3)cc2)c(N2/C(=C/C=C3\C(=O)N(C)C(=O)N3C)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C3\C(=O)N(C)C(=S)N3C)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C3C(=O)N(C)C(=O)N(C)C3=O)N(c3c(C(C)C)cccc3C(C)C)c3nc4ccccc4nc32)c(-c2ccc(-c3ccccc3)cc2)c1]

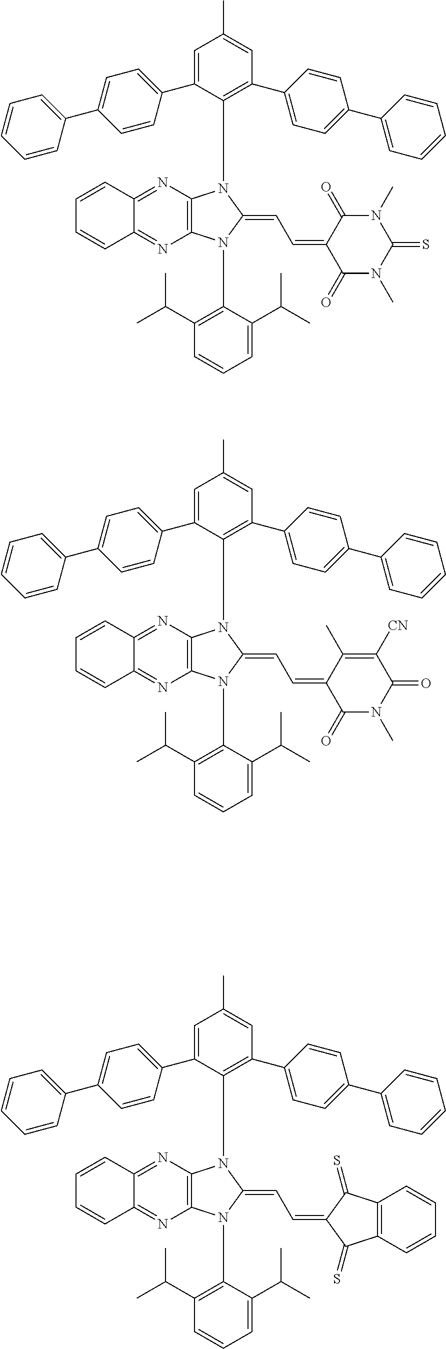 [SMILES: Cc1cc(-c2ccc(-c3ccccc3)cc2)c(N2/C(=C/C=C3C(=O)N(C)C(=S)N(C)C3=O)N(c3c(C(C)C)cccc3C(C)C)c3nc4ccccc4nc32)c(-c2ccc(-c3ccccc3)cc2)c1 ; CC1=C(C#N)C(=O)N(C)C(=O)/C1=C/C=C1/N(c2c(-c3ccc(-c4ccccc4)cc3)cc(C)cc2-c2ccc(-c3ccccc3)cc2)c2nc3ccccc3nc2N1c1c(C(C)C)cccc1C(C)C ; Cc1cc(-c2ccc(-c3ccccc3)cc2)c(N2/C(=C/C=C3C(=S)c4ccccc4C3=S)N(c3c(C(C)C)cccc3C(C)C)c3nc4ccccc4nc32)c(-c2ccc(-c3ccccc3)cc2)c1]

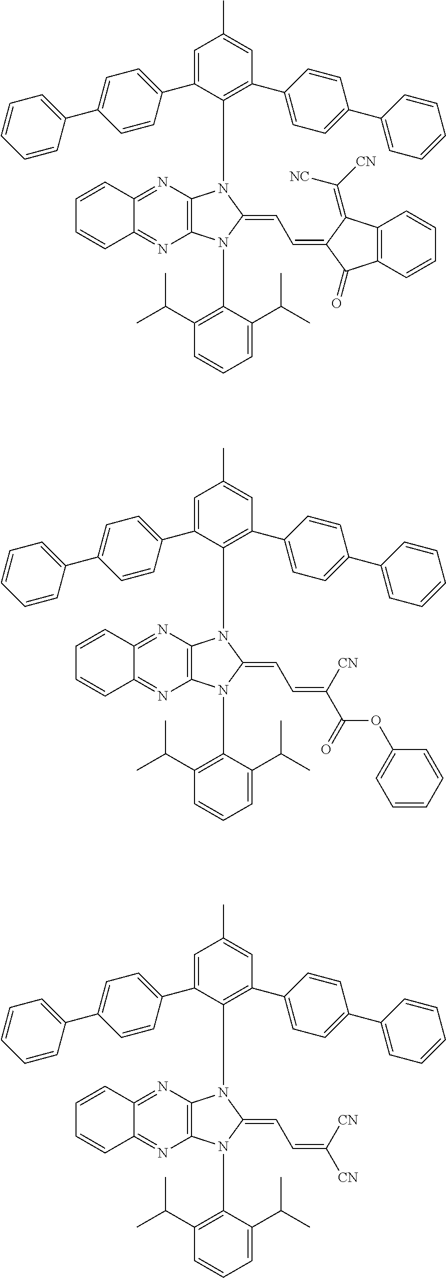 [SMILES: Cc1cc(-c2ccc(-c3ccccc3)cc2)c(N2/C(=C/C=C3/C(=O)c4ccccc4C3=C(C#N)C#N)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C(\C#N)C(=O)Oc3ccccc3)N(c3c(C(C)C)cccc3C(C)C)c3nc4ccccc4nc32)c(-c2ccc(-c3ccccc3)cc2)c1 ; Cc1cc(-c2ccc(-c3ccccc3)cc2)c(N2/C(=C/C=C(C#N)C#N)N(c3c(C(C)C)cccc3C(C)C)c3nc4ccccc4nc32)c(-c2ccc(-c3ccccc3)cc2)c1]

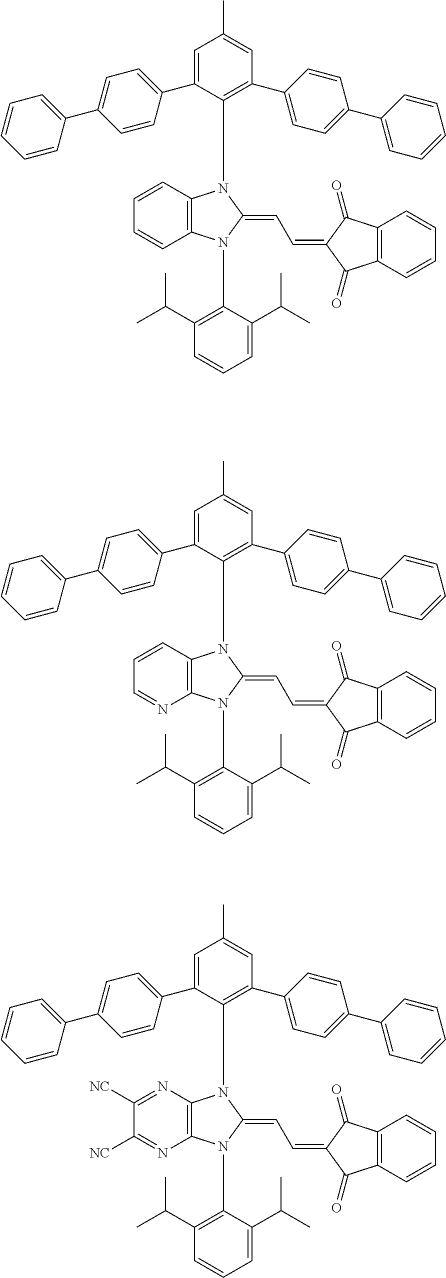 [SMILES: Cc1cc(-c2ccc(-c3ccccc3)cc2)c(N2/C(=C/C=C3C(=O)c4ccccc4C3=O)N(c3c(C(C)C)cccc3C(C)C)c3ccccc32)c(-c2ccc(-c3ccccc3)cc2)c1 ; Cc1cc(-c2ccc(-c3ccccc3)cc2)c(N2/C(=C/C=C3C(=O)c4ccccc4C3=O)N(c3c(C(C)C)cccc3C(C)C)c3ncccc32)c(-c2ccc(-c3ccccc3)cc2)c1 ; Cc1cc(-c2ccc(-c3ccccc3)cc2)c(N2/C(=C/C=C3C(=O)c4ccccc4C3=O)N(c3c(C(C)C)cccc3C(C)C)c3nc(C#N)c(C#N)nc32)c(-c2ccc(-c3ccccc3)cc2)c1]

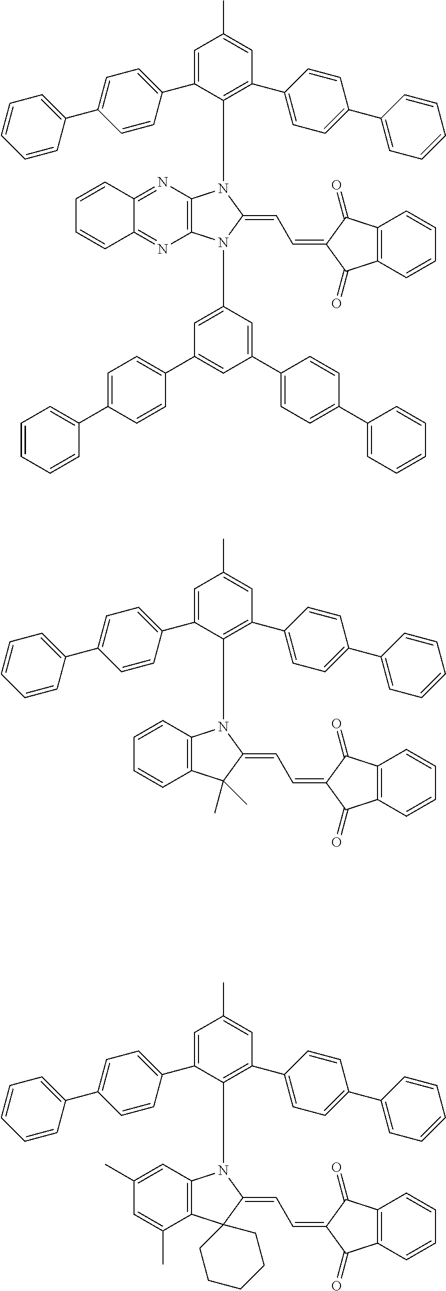 [SMILES: Cc1cc(-c2ccc(-c3ccccc3)cc2)c(N2/C(=C/C=C3C(=O)c4ccccc4C3=O)N(c3cc(-c4ccc(-c5ccccc5)cc4)cc(-c4ccc(-c5ccccc5)cc4)c3)c3nc4ccccc4nc32)c(-c2ccc(-c3ccccc3)cc2)c1 ; Cc1cc(-c2ccc(-c3ccccc3)cc2)c(N2/C(=C/C=C3C(=O)c4ccccc4C3=O)C(C)(C)c3ccccc32)c(-c2ccc(-c3ccccc3)cc2)c1 ; Cc1cc(-c2ccc(-c3ccccc3)cc2)c(N2/C(=C/C=C3C(=O)c4ccccc4C3=O)C3(CCCCC3)c3c(C)cc(C)cc32)c(-c2ccc(-c3ccccc3)cc2)c1]

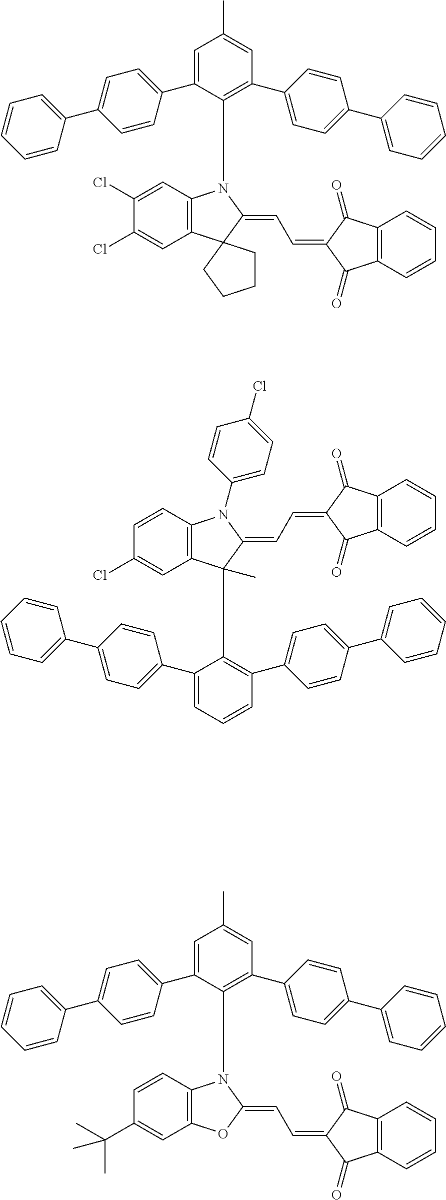 [SMILES: Cc1cc(-c2ccc(-c3ccccc3)cc2)c(N2/C(=C/C=C3C(=O)c4ccccc4C3=O)C3(CCCC3)c3cc(Cl)c(Cl)cc32)c(-c2ccc(-c3ccccc3)cc2)c1 ; CC1(c2c(-c3ccc(-c4ccccc4)cc3)cccc2-c2ccc(-c3ccccc3)cc2)/C(=C/C=C2C(=O)c3ccccc3C2=O)N(c2ccc(Cl)cc2)c2ccc(Cl)cc21 ; Cc1cc(-c2ccc(-c3ccccc3)cc2)c(N2/C(=C/C=C3C(=O)c4ccccc4C3=O)Oc3cc(C(C)(C)C)ccc32)c(-c2ccc(-c3ccccc3)cc2)c1]

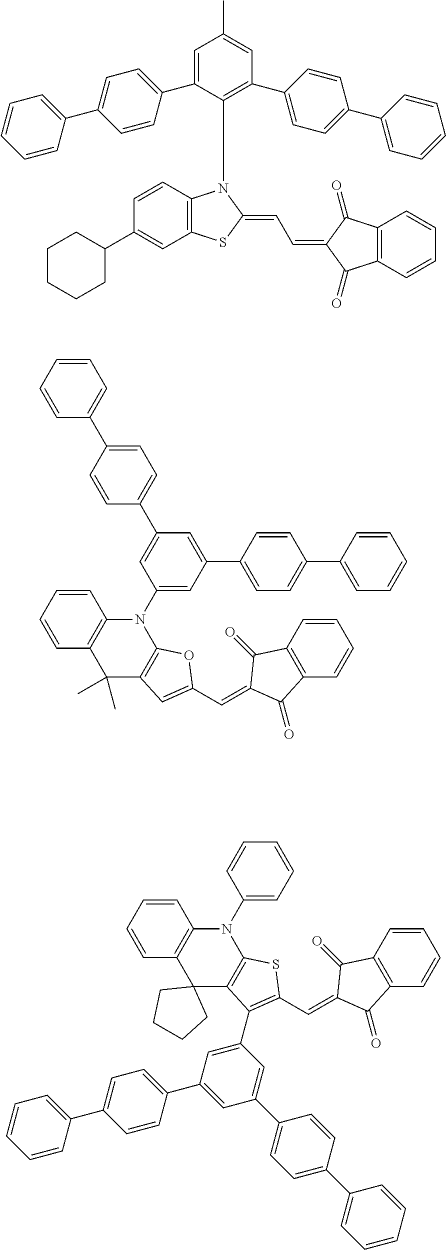 [SMILES: Cc1cc(-c2ccc(-c3ccccc3)cc2)c(N2/C(=C/C=C3C(=O)c4ccccc4C3=O)Sc3cc(C4CCCCC4)ccc32)c(-c2ccc(-c3ccccc3)cc2)c1 ; CC1(C)c2ccccc2N(c2cc(-c3ccc(-c4ccccc4)cc3)cc(-c3ccc(-c4ccccc4)cc3)c2)c2oc(C=C3C(=O)c4ccccc4C3=O)cc21 ; O=C1C(=Cc2sc3c(c2-c2cc(-c4ccc(-c5ccccc5)cc4)cc(-c4ccc(-c5ccccc5)cc4)c2)C2(CCCC2)c2ccccc2N3c2ccccc2)C(=O)c2ccccc21]

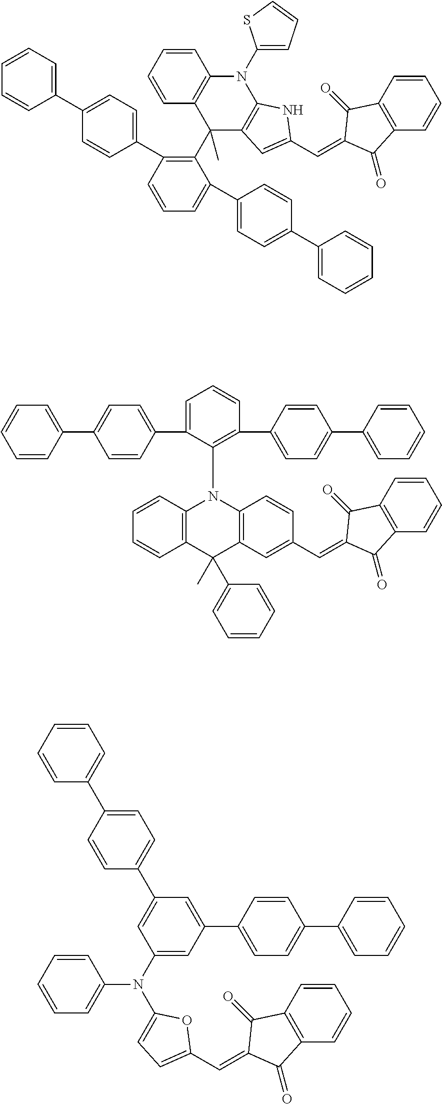 [SMILES: CC1(c2c(-c3ccc(-c4ccccc4)cc3)cccc2-c2ccc(-c3ccccc3)cc2)c2ccccc2N(c2cccs2)c2[nH]c(C=C3C(=O)c4ccccc4C3=O)cc21 ; CC1(c2ccccc2)c2ccccc2N(c2c(-c3ccc(-c4ccccc4)cc3)cccc2-c2ccc(-c3ccccc3)cc2)c2ccc(C=C3C(=O)c4ccccc4C3=O)cc21 ; O=C1C(=Cc2ccc(N(c3ccccc3)c3cc(-c4ccc(-c5ccccc5)cc4)cc(-c4ccc(-c5ccccc5)cc4)c3)o2)C(=O)c2ccccc21]

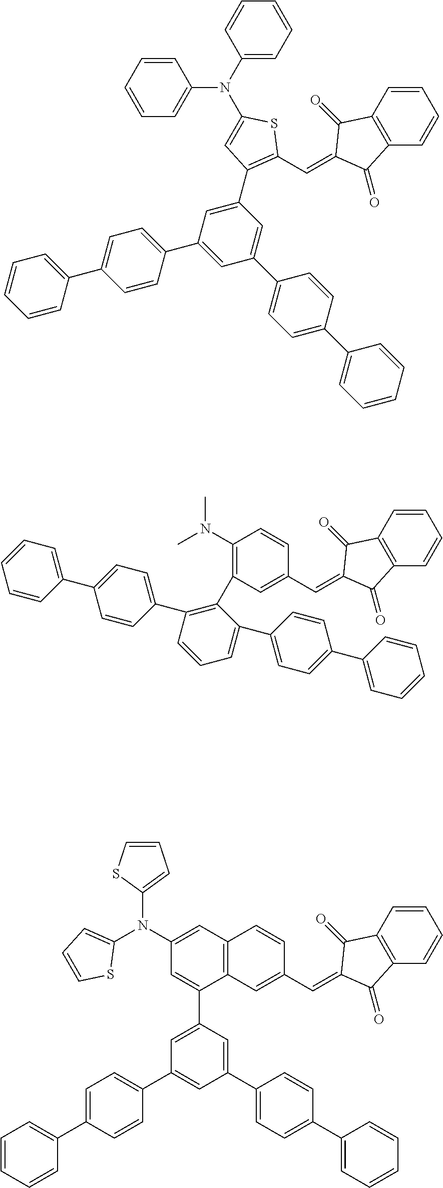 [SMILES: O=C1C(=Cc2sc(N(c3ccccc3)c3ccccc3)cc2-c2cc(-c3ccc(-c4ccccc4)cc3)cc(-c3ccc(-c4ccccc4)cc3)c2)C(=O)c2ccccc21 ; CN(C)c1ccc(C=C2C(=O)c3ccccc3C2=O)cc1-c1c(-c2ccc(-c3ccccc3)cc2)cccc1-c1ccc(-c2ccccc2)cc1 ; O=C1C(=Cc2ccc3cc(N(c4cccs4)c4cccs4)cc(-c4cc(-c5ccc(-c6ccccc6)cc5)cc(-c5ccc(-c6ccccc6)cc5)c4)c3c2)C(=O)c2ccccc21]

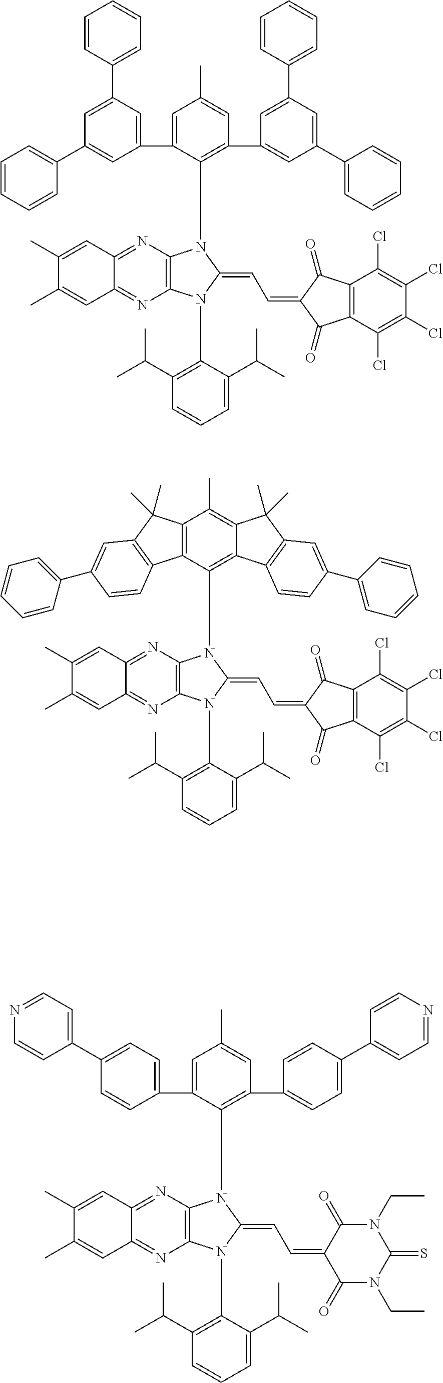 [SMILES: Cc1cc(-c2cc(-c3ccccc3)cc(-c3ccccc3)c2)c(N2/C(=C/C=C3C(=O)c4c(Cl)c(Cl)c(Cl)c(Cl)c4C3=O)N(c3c(C(C)C)cccc3C(C)C)c3nc4cc(C)c(C)cc4nc32)c(-c2cc(-c3ccccc3)cc(-c3ccccc3)c2)c1 ; Cc1cc2nc3c(nc2cc1C)N(c1c2c(c(C)c4c1-c1ccc(-c5ccccc5)cc1C4(C)C)C(C)(C)c1cc(-c4ccccc4)ccc1-2)/C(=C/C=C1C(=O)c2c(Cl)c(Cl)c(Cl)c(Cl)c2C1=O)N3c1c(C(C)C)cccc1C(C)C ; CCN1C(=O)C(=C/C=C2/N(c3c(-c4ccc(-c5ccncc5)cc4)cc(C)cc3-c3ccc(-c4ccncc4)cc3)c3nc4cc(C)c(C)cc4nc3N2c2c(C(C)C)cccc2C(C)C)C(=O)N(CC)C1=S]

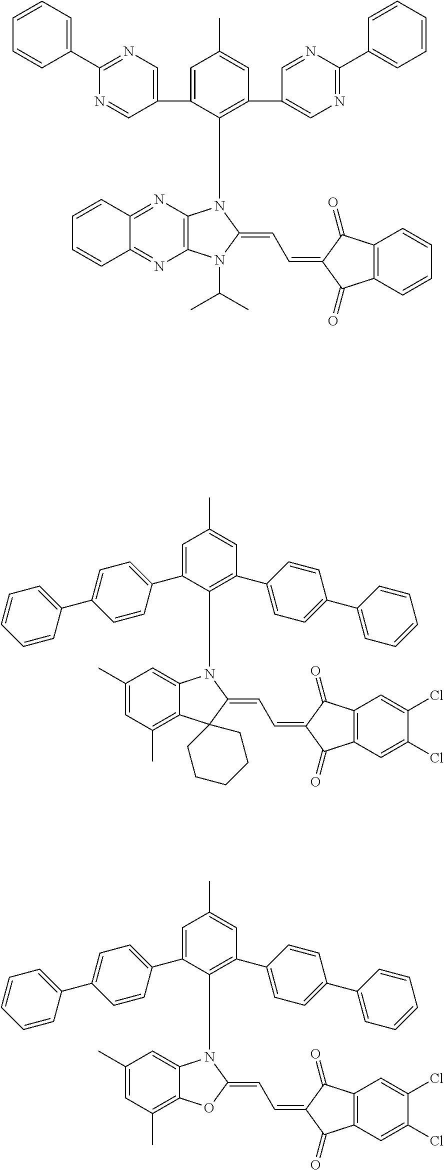 [SMILES: Cc1cc(-c2cnc(-c3ccccc3)nc2)c(N2/C(=C/C=C3C(=O)c4ccccc4C3=O)N(C(C)C)c3nc4ccccc4nc32)c(-c2cnc(-c3ccccc3)nc2)c1 ; Cc1cc(-c2ccc(-c3ccccc3)cc2)c(N2/C(=C/C=C3C(=O)c4cc(Cl)c(Cl)cc4C3=O)C3(CCCCC3)c3c(C)cc(C)cc32)c(-c2ccc(-c3ccccc3)cc2)c1 ; Cc1cc(-c2ccc(-c3ccccc3)cc2)c(N2/C(=C/C=C3C(=O)c4cc(Cl)c(Cl)cc4C3=O)Oc3c(C)cc(C)cc32)c(-c2ccc(-c3ccccc3)cc2)c1]

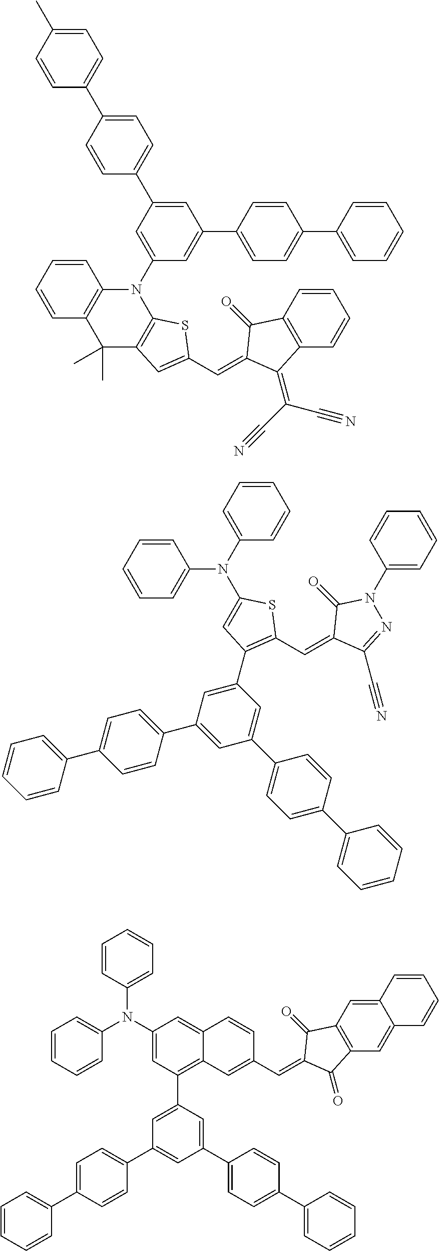

A molecular weight of the specific compound is not particularly limited, but is preferably 400 to 1,200. In a case where the molecular weight is 1,200 or less, a vapor deposition temperature is not increased, and the compound is not easily decomposed. In a case where the molecular weight is 400 or more, a glass transition point of a vapor deposition film is not lowered, and the heat resistance of the photoelectric conversion element is more improved.

The specific compound is particularly useful as a material of the photoelectric conversion film used for the imaging element, the optical sensor, or a photoelectric cell. In addition, the specific compound usually functions as the p-type organic semiconductor in the photoelectric conversion film in many cases. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −5.0 to −6.0 eV from the viewpoints of stability in a case of using the compound as the p-type organic semiconductor and matching of energy levels between the compound and the n-type organic semiconductor.

The maximum absorption wavelength of the specific compound is not particularly limited, but is preferably in the range of 500 to 600 nm, and more preferably in the range of 520 to 570 nm in that the photoelectric conversion film in the photoelectric conversion element according to the embodiment of the present invention is suitably used as an organic photoelectric conversion film that receives (absorbs) green light and performs photoelectrically conversion.

An absorption half-width of the specific compound is not particularly limited, but is preferably 120 nm or less, more preferably 95 nm or less, still more preferably 90 nm or less, and particularly preferably 85 nm or less in that the photoelectric conversion film in the photoelectric conversion element according to the embodiment of the present invention is suitably used as an organic photoelectric conversion film that receives (absorbs) green light and is photoelectrically converted. The lower limit is not particularly limited, but is often 60 nm or more.

The maximum absorption wavelength and the absorption half-width are values measured in a film state of the specific compound (for example, a vapor deposition film of the specific compound).

The maximum absorption wavelength of the photoelectric conversion film is not particularly limited, but is preferably in the range of 500 to 600 nm, and more preferably in the range of 520 to 570 nm in that the photoelectric conversion film in the photoelectric conversion element according to the embodiment of the present invention is suitably used as an organic photoelectric conversion film that receives (absorbs) green light and is photoelectrically converted.

<n-Type Organic Semiconductor>

It is preferable that the photoelectric conversion film contains the n-type organic semiconductor as a component other than the specific compound.

The n-type organic semiconductor is an acceptor-property organic semiconductor material (a compound), and refers to an organic compound having a property of easily accepting an electron. More specifically, the n-type organic semiconductor refers to an organic compound having a large electron affinity of two organic compounds used in contact with each other. Therefore, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative; oxadiazole derivative; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

Among these, it is preferable that examples of the n-type organic semiconductor (compound) include fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

An organic coloring agent may be used as the n-type organic semiconductor. Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1200, and more preferably 200 to 900.

From the point that the photoelectric conversion film in the photoelectric conversion element according to the embodiment of the present invention is suitably used as an organic photoelectric conversion film that receives (absorbs) green light and is photoelectrically converted, it is preferable that the n-type organic semiconductor is colorless or has a maximum absorption wavelength and/or an absorption waveform close to that of the specific compound, and as the specific value, the maximum absorption wavelength of the n-type organic semiconductor is preferably 400 nm or less or in the range of 500 to 600 nm.

It is preferable that the photoelectric conversion film has a bulk hetero structure formed in a state in which the specific compound and the n-type organic semiconductor are mixed with each other. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A and the like.

From the viewpoint of responsiveness of the photoelectric conversion element, the content of the specific compound to the total content of the specific compound and the n-type organic semiconductor (=film thickness in terms of single layer of specific compound/(film thickness in terms of single layer of specific compound+film thickness in terms of single layer of n-type organic semiconductor)×100) is preferably 20% to 80% by volume, and more preferably 40% to 80% by volume.

Also, in a case where the photoelectric conversion film contains a p-type organic semiconductor described below, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor)×100) is preferably 15% to 75% by volume, and more preferably 30% to 75% by volume.

It is preferable that the photoelectric conversion film is substantially formed of the specific compound, the n-type organic semiconductor, and the p-type organic semiconductor included as desired. The term “substantially” means that a total content of the specific compound, the n-type organic semiconductor, and the p-type organic semiconductor included as desired is 95% by mass or more with respect to a total mass of the photoelectric conversion film.

The n-type organic semiconductor contained in the photoelectric conversion film may be used alone or in combination of two or more.

In addition to the specific compound and the n-type organic semiconductor, the photoelectric conversion film may further contain the p-type organic semiconductor. Examples of the p-type organic semiconductor include the following compounds.

The p-type organic semiconductor here means a p-type organic semiconductor which is a compound different from the specific compound. In a case where the photoelectric conversion film contains the p-type organic semiconductor, the p-type organic semiconductor may be used alone or in combination of two or more.

<p-Type Organic Semiconductor>

The p-type organic semiconductor is a donor organic semiconductor material (a compound), and refers to an organic compound having a property of easily donating an electron. More specifically, the p-type organic semiconductor means an organic compound having a smaller ionization potential in a case where two organic compounds are used in contact with each other.

Examples of the p-type organic semiconductor include triarylamine compounds (for example, N, N′-bis(3-methylphenyl)-(1,1′-biphenyl)-4,4′-diamine (TPD), 4,4′-bis[N-(naphthyl)-N-Phenyl-amino]biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-094660A, pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b][1]benzothiophene (BTBT) derivative, a thieno[3,2-f: 4,5-f′]bis[1]benzothiophene (TBBT) derivative, compounds disclosed in paragraphs [0031] to [0036] of JP2018-014474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-054228A, compounds disclosed in paragraphs [0063] to [0089] of WO2019-058995A, compounds disclosed in paragraphs [0063] to [0089] of WO2019-081416A, compounds disclosed in paragraphs [0033] to [0036] of JP2019-080052A, compounds disclosed in paragraphs [0044] to [0054] of WO2019-054125A, compounds disclosed in paragraphs [0041] to [0046] of WO2019-093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, and a fluoranthene derivative), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands. Examples of the p-type organic semiconductor include compounds having an ionization potential smaller than that of the n-type organic semiconductor, and in a case where this condition is satisfied, the organic dyes exemplified as the n-type organic semiconductor can be used.

The compounds that can be used as the p-type semiconductor compound are exemplified below.

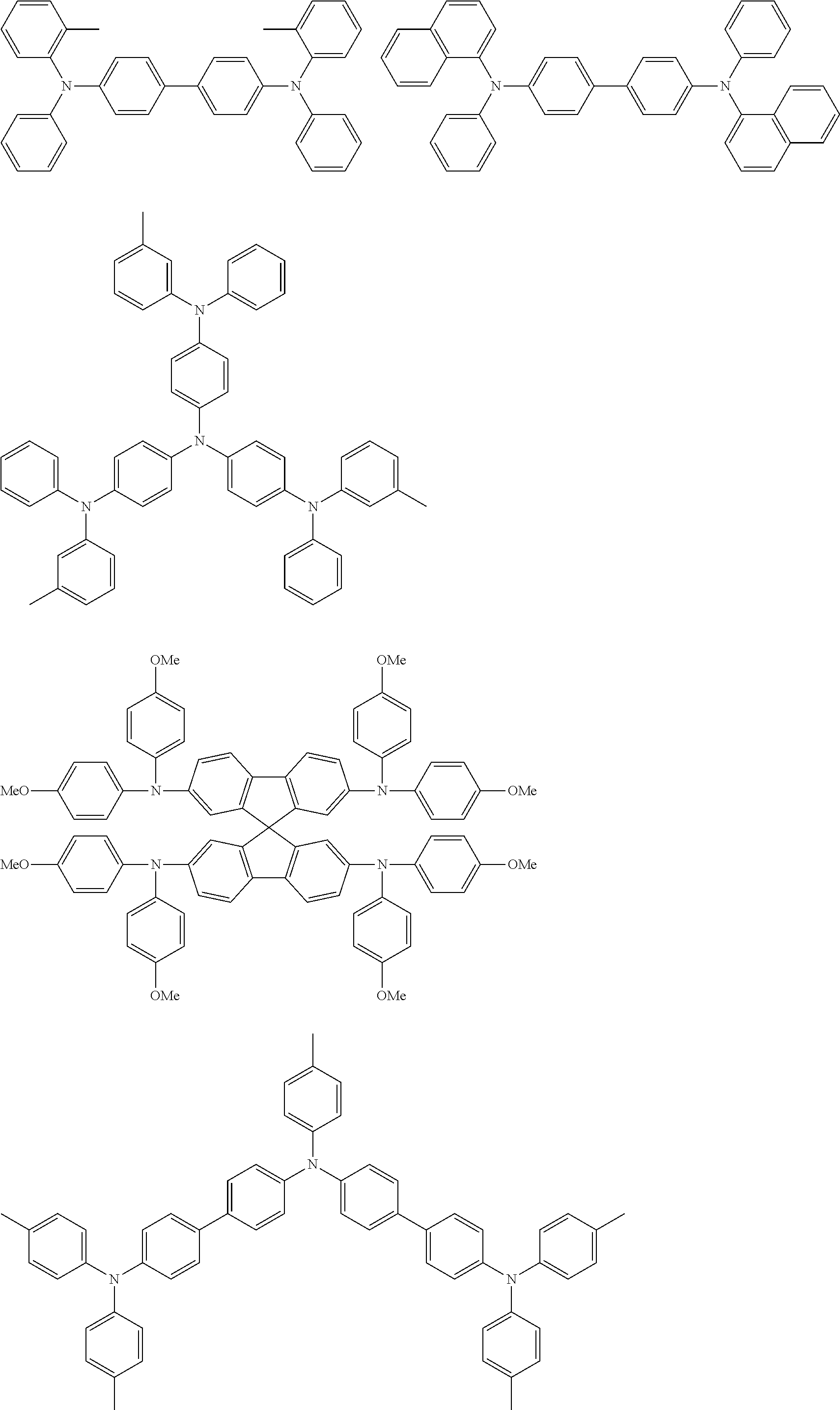

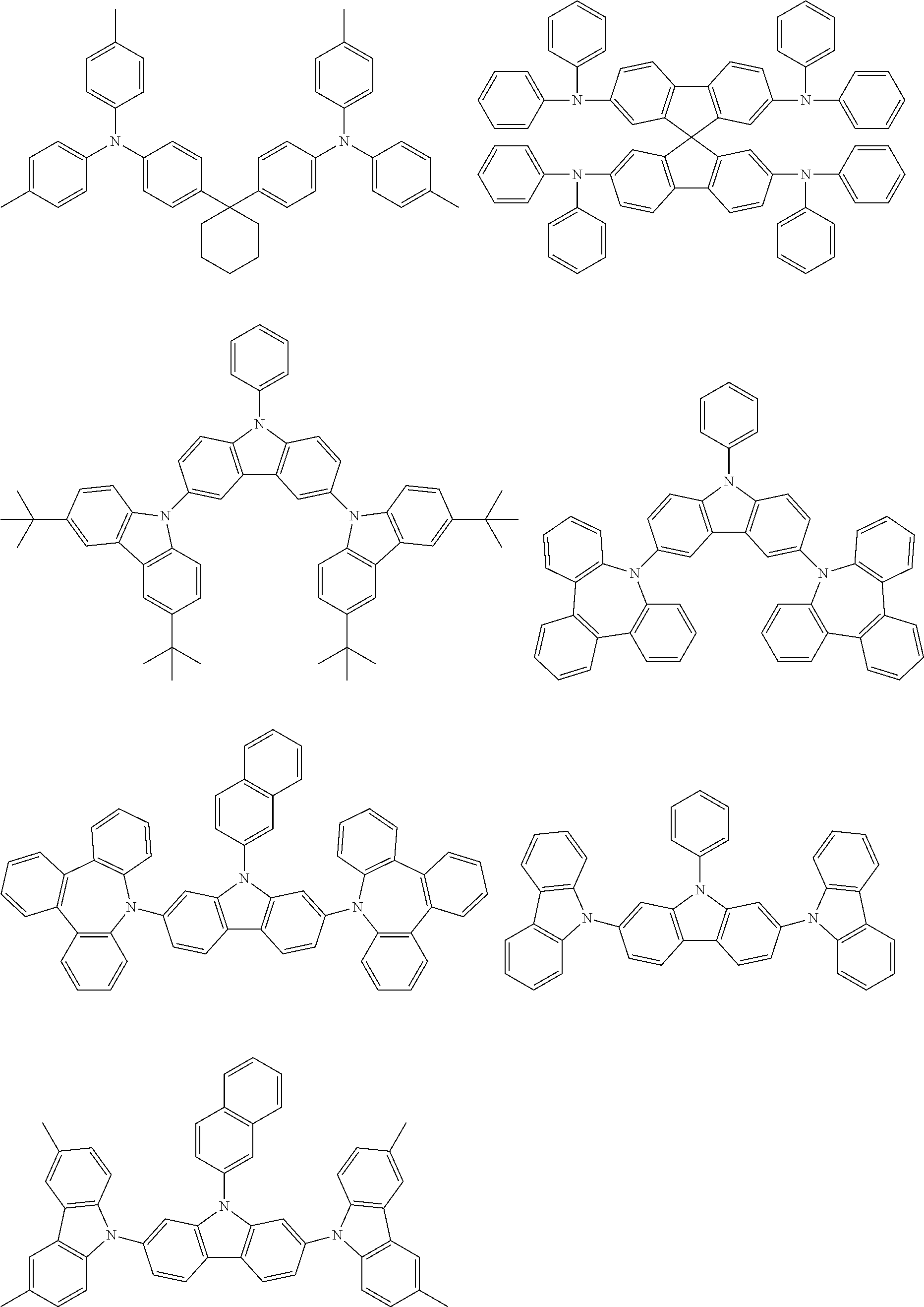

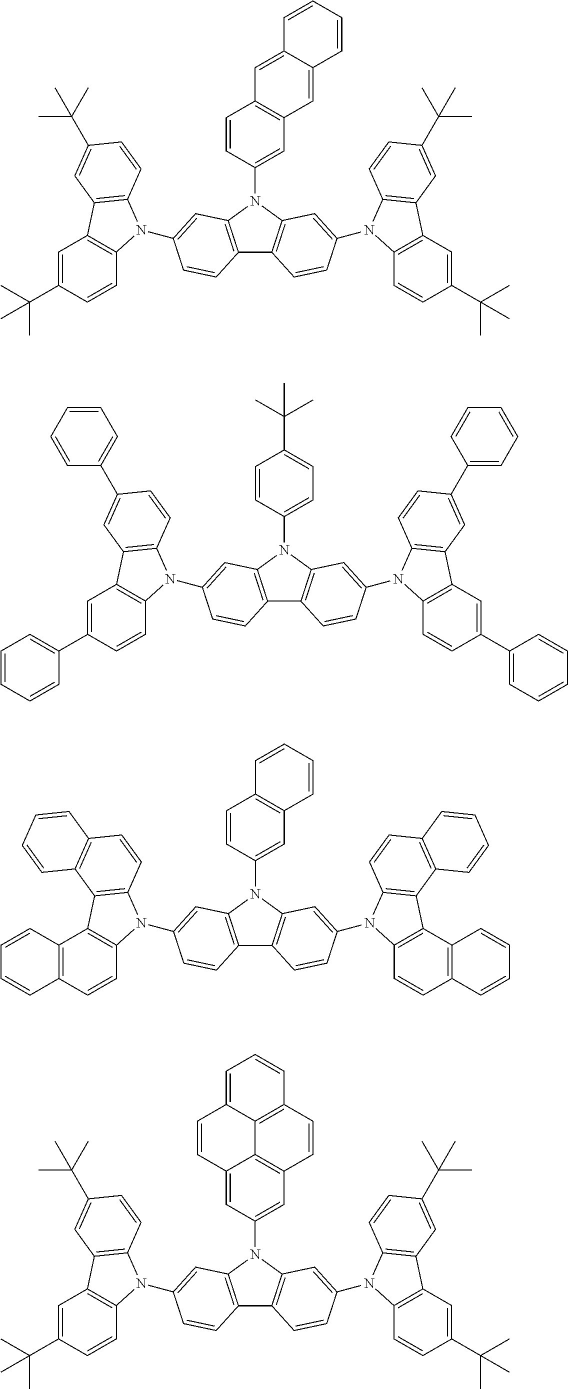

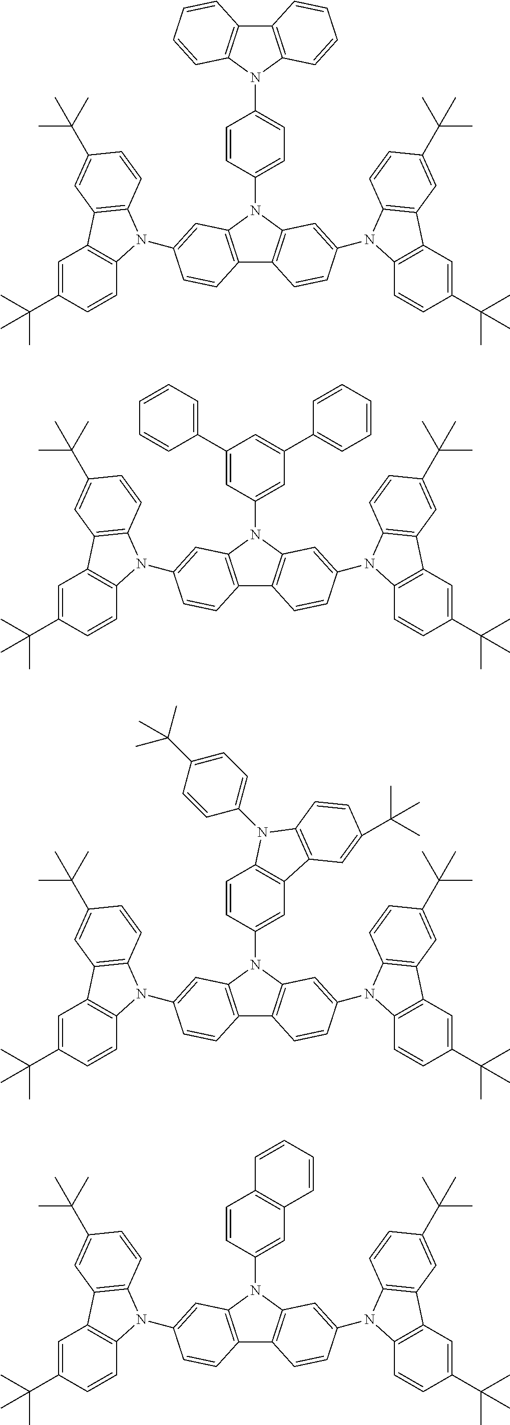

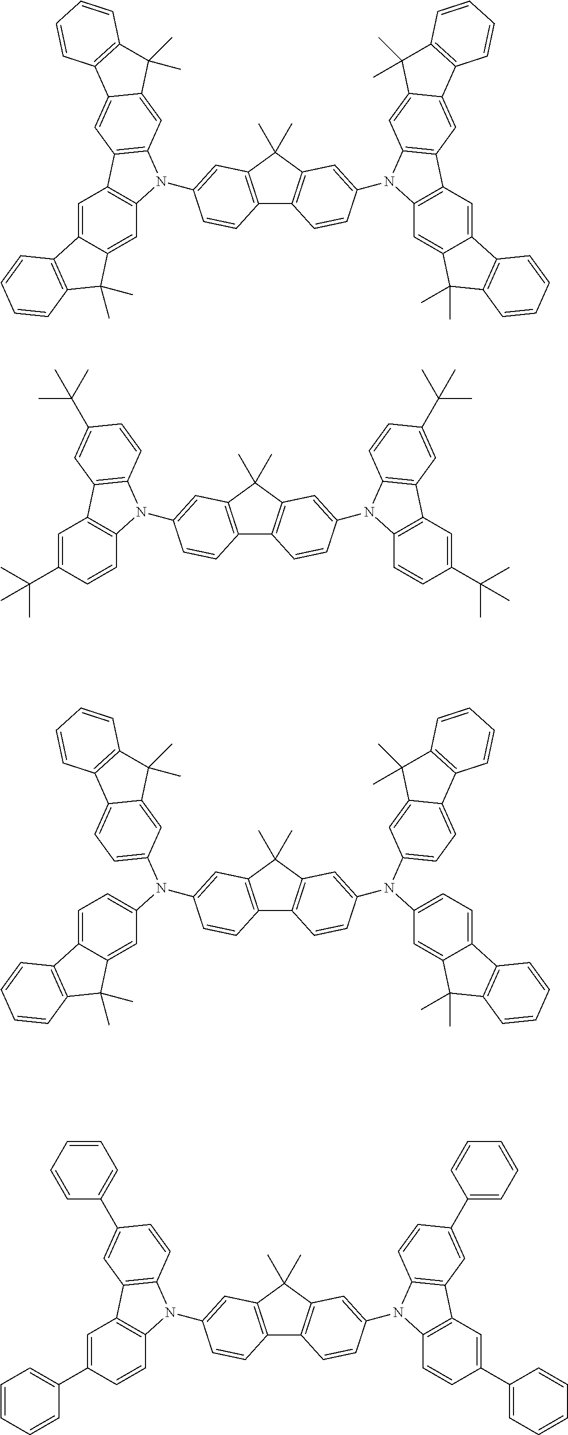

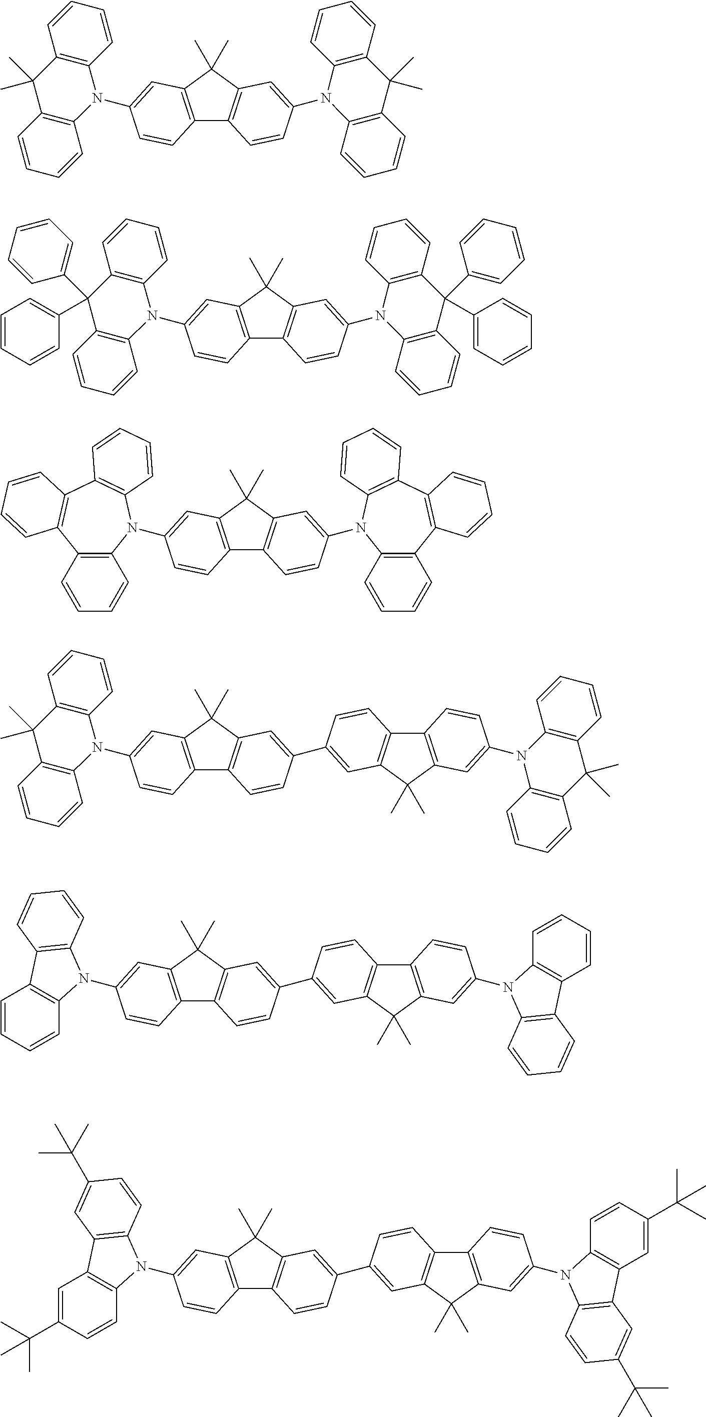

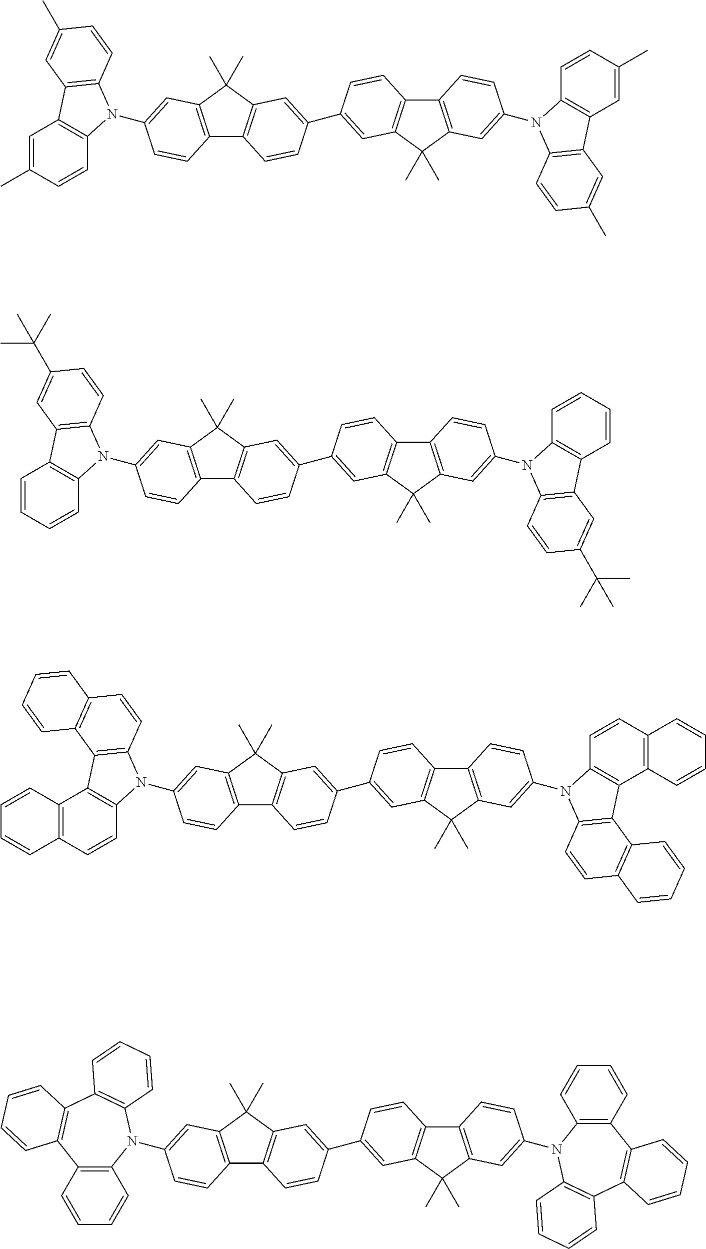

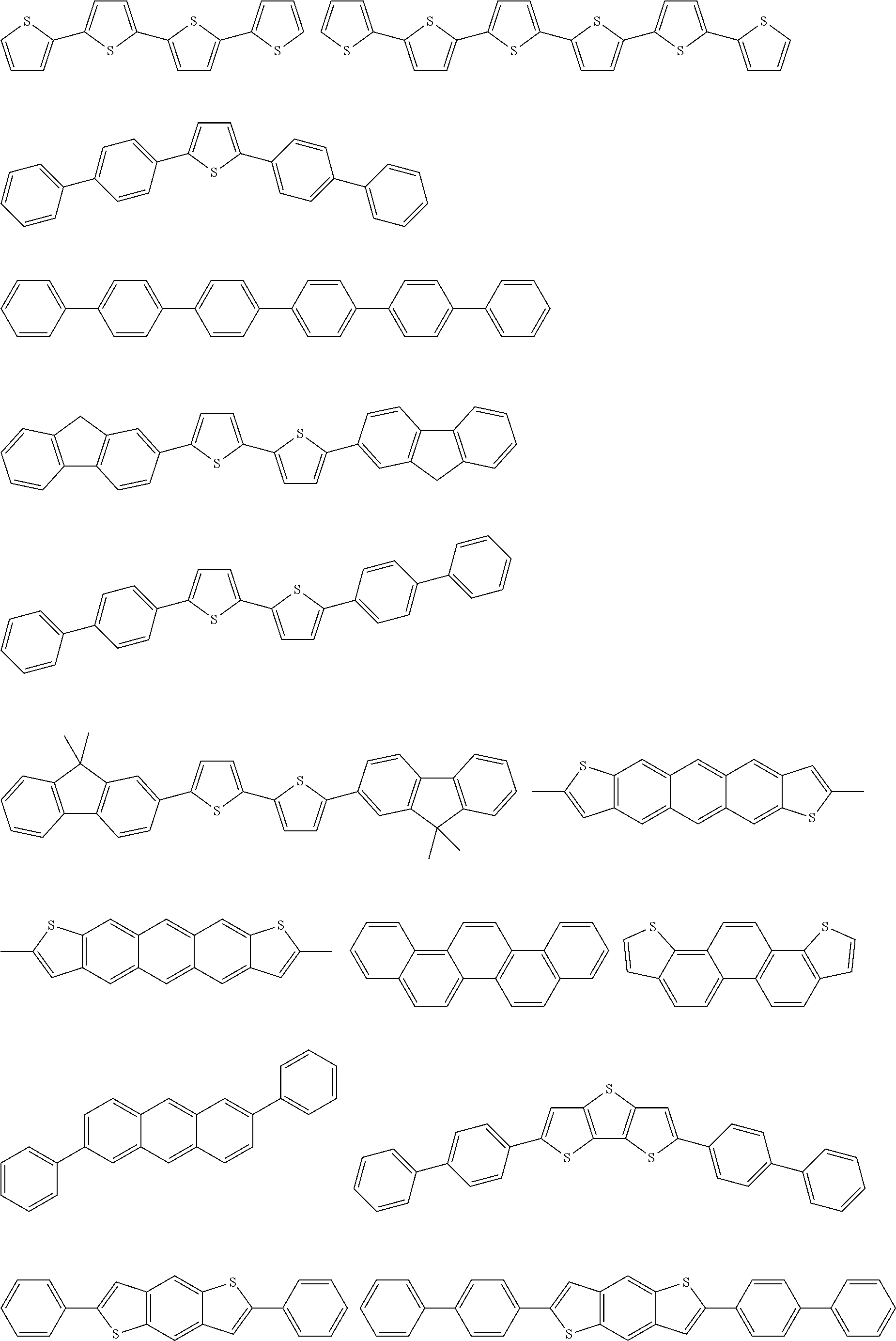

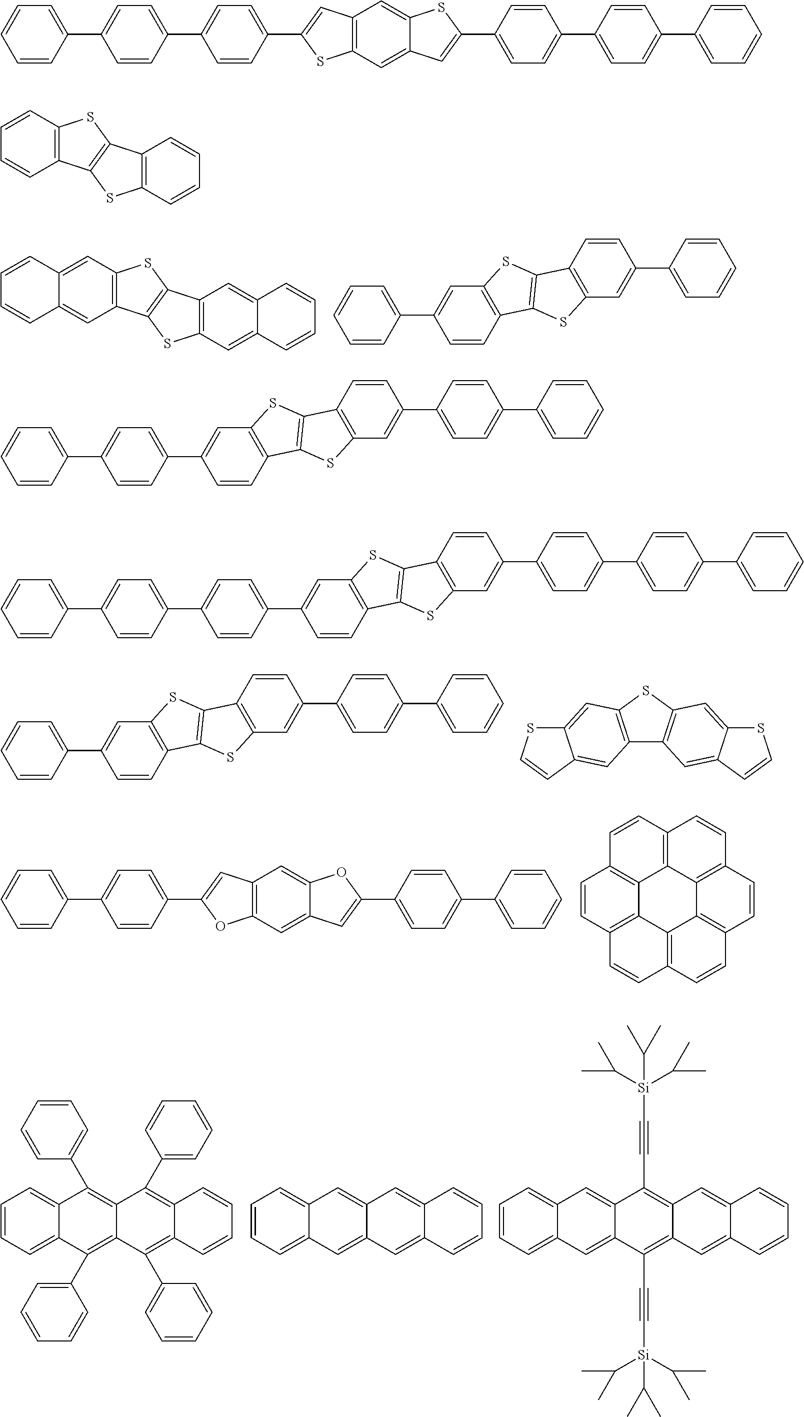

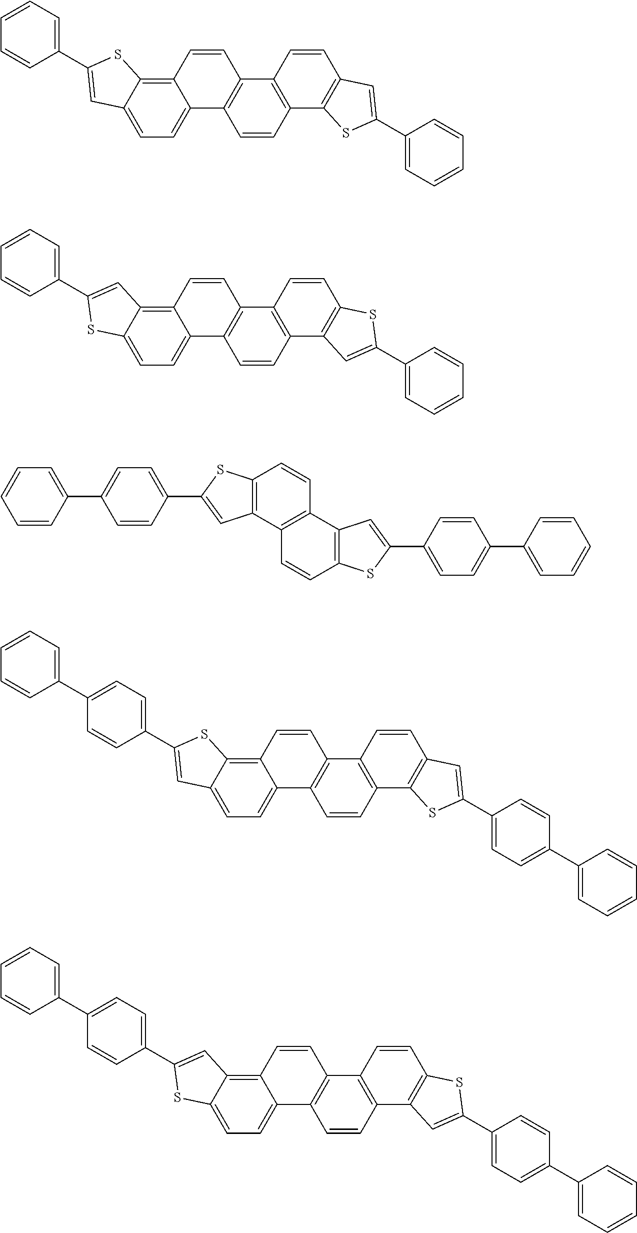 [SMILES: c1ccc(-c2cc3ccc4c5ccc6c(ccc7cc(-c8ccccc8)sc76)c5ccc4c3s2)cc1 ; c1ccc(-c2cc3c(ccc4c3ccc3c5ccc6sc(-c7ccccc7)cc6c5ccc43)s2)cc1 ; c1ccc(-c2ccc(-c3cc4c(ccc5c6cc(-c7ccc(-c8ccccc8)cc7)sc6ccc45)s3)cc2)cc1 ; c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1]

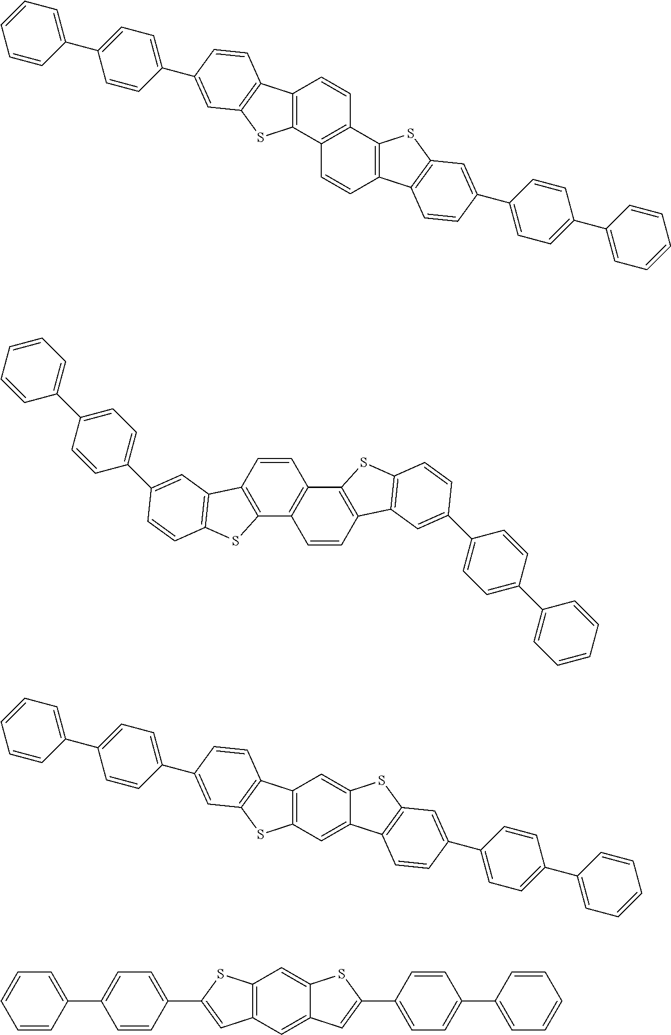 [SMILES: c1ccc(-c2ccc(-c3ccc4c(c3)sc3c4ccc4c3ccc3c5ccc(-c6ccc(-c7ccccc7)cc6)cc5sc34)cc2)cc1 ; c1ccc(-c2ccc(-c3ccc4sc5c(ccc6c5ccc5c7cc(-c8ccc(-c9ccccc9)cc8)ccc7sc56)c4c3)cc2)cc1 ; c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3cc4cc5cc(-c6ccc(-c7ccccc7)cc6)sc5cc4s3)cc2)cc1]

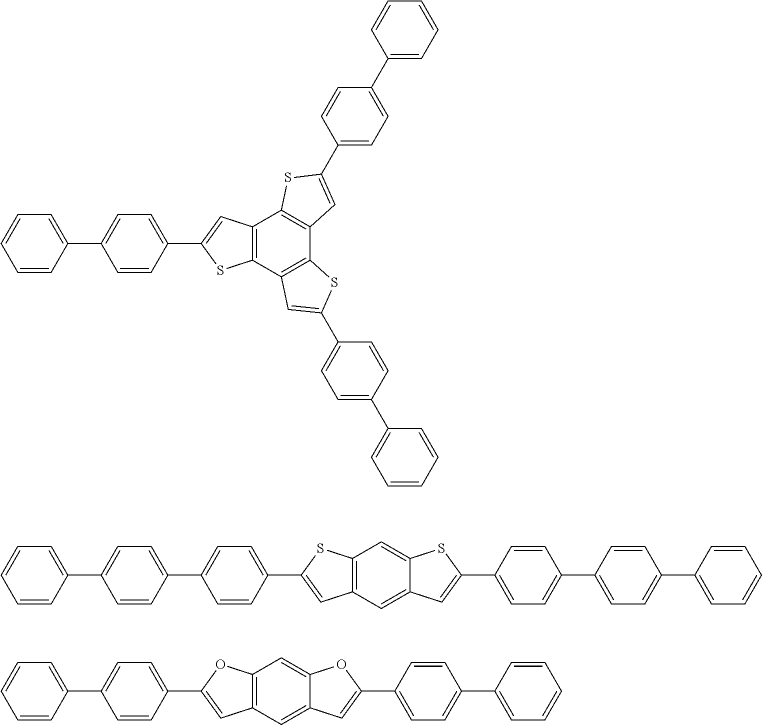 [SMILES: c1ccc(-c2ccc(-c3cc4c(s3)c3cc(-c5ccc(-c6ccccc6)cc5)sc3c3cc(-c5ccc(-c6ccccc6)cc5)sc43)cc2)cc1 ; c1ccc(-c2ccc(-c3ccc(-c4cc5cc6cc(-c7ccc(-c8ccc(-c9ccccc9)cc8)cc7)sc6cc5s4)cc3)cc2)cc1 ; c1ccc(-c2ccc(-c3cc4cc5cc(-c6ccc(-c7ccccc7)cc6)oc5cc4o3)cc2)cc1]

The photoelectric conversion film containing the specific compound is a non-light emitting film, and has a feature different from organic light emitting diodes (OLEDs). The non-light emitting film is intended for a film having a light emission quantum efficiency of 1% or less, and the light emission quantum efficiency is preferably 0.5% or less, and more preferably 0.1% or less.

### <Film Formation Method>

The photoelectric conversion film can be formed mostly by a dry film formation method. Examples of the dry film formation method include a physical vapor deposition method such as a vapor deposition method (in particular, a vacuum vapor deposition method), a sputtering method, and an ion plating method, a molecular beam epitaxy (MBE) method, and a chemical vapor deposition (CVD) method such as plasma polymerization. Among these, the vacuum vapor deposition method is preferable. In a case where the photoelectric conversion film is formed by the vacuum vapor deposition method, manufacturing conditions such as a degree of vacuum and a vapor deposition temperature can be set according to the normal method.

The thickness of the photoelectric conversion film is preferably 10 to 1000 nm, more preferably 50 to 800 nm, still more preferably 50 to 500 nm, and particularly preferably 50 to 300 nm.

### <Electrode>

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the material forming the upper electrode 15 include conductive metal oxides such as tin oxide (antimony tin oxide (ATO), fluorine doped tin oxide (FTO)) doped with antimony, fluorine, or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is preferably 100 to 10000Ω/□, and a degree of freedom of a range of the film thickness that can be thinned is large. In addition, as the thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, conductive compounds (for example, titanium nitride (TiN)) such as oxides or nitrides of these metals; mixtures or laminates of these metals and conductive metal oxides; and organic conductive materials such as polyaniline, polythiophene, and polypyrrole.

The method of forming electrodes is not particularly limited, and can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### <Charge Blocking Film: Electron Blocking Film and Positive Hole Blocking Film>

It is also preferable that the photoelectric conversion element according to the embodiment of the present invention has one or more interlayers between the conductive film and the transparent conductive film, in addition to the photoelectric conversion film. Example of the interlayer includes the charge blocking film. In a case where the photoelectric conversion element has this film, the characteristics (such as photoelectric conversion efficiency and responsiveness) of the photoelectric conversion element to be obtained is more excellent. Examples of the charge blocking film include the electron blocking film and the positive hole blocking film. Hereinafter, each of the films will be described in detail.

### (Electron Blocking Film)

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### (Positive Hole Blocking Film)

A Positive hole blocking film is an acceptor-property organic semiconductor material (a compound), and the n-type semiconductor described above can be used.

The method of producing the charge blocking film is not particularly limited, but a dry film formation method and a wet film formation method are exemplified. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each thickness of the charge blocking films (the electron blocking film and the positive hole blocking film) is preferably 3 to 200 nm, more preferably 5 to 100 nm, and still more preferably 5 to 30 nm.

### <Substrate>

The photoelectric conversion element may further include a substrate. The type of substrate to be used is not particularly limited, but a semiconductor substrate, a glass substrate, and a plastic substrate are exemplified.

The position of the substrate is not particularly limited, but in general, the conductive film, the photoelectric conversion film, and the transparent conductive film are laminated on the substrate in this order.

### <Sealing Layer>

The photoelectric conversion element may further include a sealing layer. The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be produced according to the description in paragraphs [0210] to [0215] of JP2011-082508A.

### [Imaging Element]

An example of the application of the photoelectric conversion element includes an imaging element. The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

FIG. 3 is a schematic cross-sectional view showing a schematic configuration of an imaging element for describing an embodiment of the present invention. This imaging element is mounted on an imaging element such as a digital camera and a digital video camera, an electronic endoscope, and imaging modules such as a cellular phone.

An imaging element 20a shown in FIG. 3 includes a photoelectric conversion element 10a according to the embodiment of the present invention, a blue photoelectric conversion element 22, and a red photoelectric conversion element 24, which are laminated along the light incident direction. As described above, the photoelectric conversion element 10a can mainly function as a green photoelectric conversion element capable of receiving green light.

The imaging element 20a is a so-called laminated-type color separation imaging element. The photoelectric conversion element 10a, the blue photoelectric conversion element 22, and the red photoelectric conversion element 24 have different wavelength spectra to be detected. That is, the blue photoelectric conversion element 22 and the red photoelectric conversion element 24 correspond to photoelectric conversion elements that receive (absorb) light having a wavelength different from a wavelength of light received by the photoelectric conversion element 10a. The photoelectric conversion element 10a can mostly receive green light, the blue photoelectric conversion element 22 can mostly receive blue light, and the red photoelectric conversion element can mostly receive red light.

Green light means light in a wavelength range of 500 to 600 nm, blue light means light in a wavelength range of 400 to 500 nm, and red light means light in a wavelength range of 600 to 700 nm.

In a case where light is incident on the imaging element 20a in the direction of the arrow, firstly, green light is mostly absorbed by the photoelectric conversion element 10a, but blue light and red light are transmitted through the photoelectric conversion element 10a. In a case where the light transmitted through the photoelectric conversion element 10a travels to the blue photoelectric conversion element 22, the blue light is mostly absorbed, but the red light is transmitted through the blue photoelectric conversion element 22. Thereafter, light transmitted through the blue photoelectric conversion element 22 is absorbed by the red photoelectric conversion element 24. As described above, in the imaging element 20a, which is a laminated type color separation imaging element, one pixel can be configured with three light receiving sections of green, blue, and red, and a large area of the light receiving section can be taken.

In particular, the photoelectric conversion element 10a according to the embodiment of the present invention has a narrow absorption peak half-width, and thus absorptions of blue light and red light do not occur, and it is difficult to affect the detectability of the blue photoelectric conversion element 22 and the red photoelectric conversion element 24.

The configurations of the blue photoelectric conversion element 22 and the red photoelectric conversion element 24 are not particularly limited.

For example, the photoelectric conversion element having a configuration in which colors are separated by using silicon due to a difference in light absorption length may be used. As a more specific example, both the blue photoelectric conversion element 22 and the red photoelectric conversion element 24 may be made of silicon. In this case, as for light including blue light, green light, and red light that has entered the imaging element 20a in the direction of the arrow, the photoelectric conversion element 10a mostly receives the green light having the center wavelength, and the remaining blue light and red light are easily separated. The blue light and red light have different light absorption lengths for silicon (wavelength dependence of absorption coefficient for silicon), the blue light is easily absorbed near a surface of silicon, and the red light can penetrate deeper into the silicon. Based on such a difference in light absorption length, the blue light is mostly received by the blue photoelectric conversion element 22 existing in a shallower position, and the red light is mostly received by the red photoelectric conversion element 24 existing in a deeper position.

In addition, the blue photoelectric conversion element 22 and the red photoelectric conversion element 24 each may be a photoelectric conversion element (the blue photoelectric conversion element 22 or the red photoelectric conversion element 24) having a configuration including a conductive film, an organic photoelectric conversion film having an absorption maximum for blue light or red light, and a transparent conductive film in this order.

In FIG. 3, the photoelectric conversion element according to the embodiment of the present invention, the blue photoelectric conversion element, and the red photoelectric conversion element are arranged in this order from the light incident side, but the arrangement is not limited to the aspect, and may be another aspect. For example, the blue photoelectric conversion element, the photoelectric conversion element according to the embodiment of the present invention, and the red photoelectric conversion element may be arranged in this order from the light incident side.

As described above, the configuration in which the photoelectric conversion elements of the three primary colors of blue, green, and red are laminated as the imaging element is described, but the configuration may be two layers (two colors) or four layers (four colors) or more.

For example, an aspect in which the photoelectric conversion element 10a according to the embodiment of the present invention may be arranged on the arrayed blue photoelectric conversion element 22 and red photoelectric conversion element 24 may be employed. As needed, a color filter that further absorbs light of a predetermined wavelength may be arranged on the light incident side.

The form of the imaging element is not limited to the above-described form and the form shown in FIG. 3 and may be other forms.

For example, an aspect in which the photoelectric conversion element according to the embodiment of the present invention, the blue photoelectric conversion element, and the red photoelectric conversion element may be arranged in the same plane position may be employed.

In addition, the photoelectric conversion element may be used as a single layer. For example, a configuration in which blue, red, and green color filters are arranged on the photoelectric conversion element 10a according to the embodiment of the present invention to separate colors may be employed.

### [Optical Sensor]

Examples of another application of the photoelectric conversion element include the photoelectric cell and the optical sensor, but the photoelectric conversion element according to the embodiment of the present invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention further includes the invention of compounds. A compound according to the embodiment of the present invention is the same as the compound represented by Formula (1).

### Examples

Hereinafter, the present invention will be described in more detail based on Examples. The materials, the amount and ratio of the materials used, how to treat the materials, the treatment procedure, and the like shown in the following examples can be appropriately changed as long as the gist of the present invention is maintained. Accordingly, the scope of the present invention should not be construed as being limited to Examples shown below.

### [Compound (Evaluation Compound)]

### [Synthesis Method of Compound (D-1)]

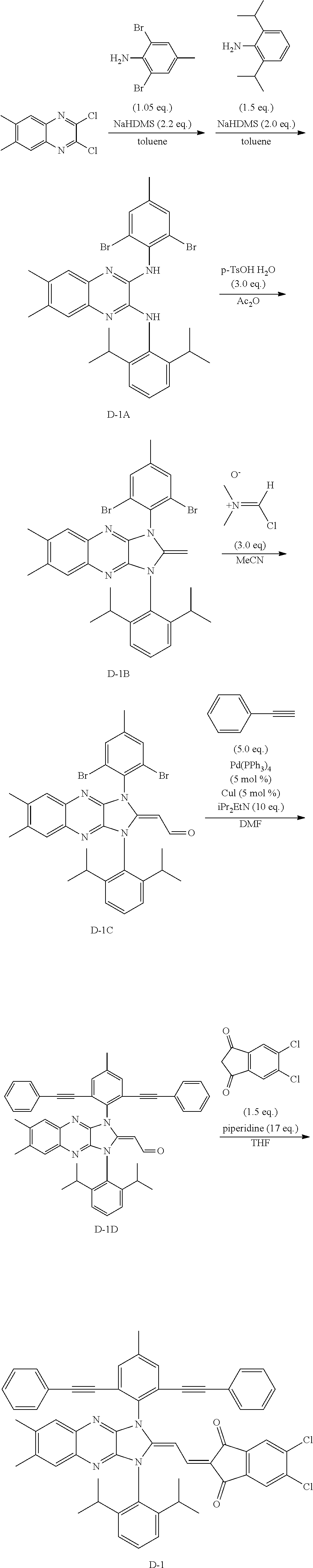

Under a nitrogen atmosphere, 2,3-dichloro-6,7-dimethylquinoxaline (10.00 g, 44.0 mmol), 2,6-dibromo-4-methylaniline (12.25 g, 46.2 mmol), and toluene (59 mL) were added to a 500 mL three-neck flask, and 51.0 mL of a 1.9 M tetrahydrofuran solution of sodium bis(trimethylsilyl)amide (NHMDS) (96.9 mmol) was added dropwise thereto at room temperature. Subsequently, 2,6-diisopropylaniline (12.13 mL, 64.3 mmol) was added thereto while cooling with ice, and 67.0 mL of a 1.9 M tetrahydrofuran solution of sodium bis(trimethylsilyl)amide (NHMDS) was added dropwise thereto (127.3 mmol). The mixture was heated to room temperature and reacted for 1 hour. 100 ml of water, 50 mL of saturated saline, and 100 mL of ethyl acetate were added to the reaction solution, the mixture was extracted and separated, and the obtained organic layer was dried over anhydrous magnesium sulfate and concentrated under reduced pressure to obtain 25.0 g of Compound D-1A (41.9 mmol, yield of 95%).

Under a nitrogen atmosphere, 25.0 g of Compound D-1A (41.9 mmol) and 42 mL of acetic acid anhydride was added to a 500 mL three-neck flask, 23.9 g of p-toluenesulfonic acid monohydrate (126 mmol) was slowly added thereto in several portions at room temperature with stirring, and the mixture was stirred at 115° C. for 2 hours. The reaction solution was slowly added dropwise to a mixture of 95 g of sodium hydroxide, which had been separately prepared, and 375 g of ice, and the mixture was stirred at room temperature, and the precipitated solid was collected by filtration. The crude product was purified by silica gel column chromatography (developing solvent: toluene) to obtain 17.1 g of Compound D-1B (27.6 mmol, yield of 66%).

Under a nitrogen atmosphere, 10.5 g of (chloromethylene)dimethylammonium chloride (Vilsmeier reagent) (82.2 mmol) and 170 mL of acetonitrile were added to a 500 mL three-neck flask, 17.0 g of Compound D-1B (27.4 mmol) was slowly added thereto at room temperature with stirring, and the mixture was further stirred at room temperature for 2 hours. The reaction solution was slowly added dropwise to a mixture of 10 g of sodium hydroxide, which had been separately prepared, and 500 g of ice, and the mixture was stirred at room temperature, and the precipitated solid was collected by filtration. The crude product was recrystallized with dichloromethane/acetonitrile to obtain 11.3 g of Compound D-1C (17.4 mmol, yield of 63%).

Under a nitrogen atmosphere, 5.00 g of Compound D-1C (7.71 mmol), 3.94 g of phenylacetylene (38.6 mmol), 73.4 mg of cuprous iodide (I) (0.386 mmol), 77.1 mL of N,N-dimethylformamide, and 13.5 mL of diisopropylethylamine were added to a 200 mL three-neck flask, 446 mg of tetrakis(triphenylphosphine)palladium (0.386 mmol) was added thereto while stirring at room temperature, and then the mixture was stirred at 90° C. for 4 hours. After being allowed to cool to room temperature, 50 mL of water and 50 mL of ethyl acetate were added thereto, the mixture was extracted and separated, and the obtained organic layer was dried over anhydrous magnesium sulfate. The crude product obtained by concentrating was purified by silica gel column chromatography (developing solvent: toluene/ethyl acetate) to obtain 2.88 g of Compound D-1D (4.17 mmol, yield of 54%).

Under a nitrogen atmosphere, 1.00 g of Compound D-1D (1.45 mmol), 467 mg of 5,6-dichloroindandione (2.17 mmol), 10 mL of tetrahydrofuran, and 2.4 mL of piperidine were added to a 100 mL three-neck flask, and the mixture was stirred at 50° C. for 3 hours. The mixture was cooled to room temperature, 30 mL of methanol was added thereto, and the precipitated solid was collected by filtration and washed with methanol to obtain 1.03 g of Compound D-1 (1.16 mmol, yield of 80%).

The obtained compound (D-1) was identified by nuclear magnetic resonance (NMR) and mass spectrometry (MS).

¹H NMR spectrum (400 MHZ, CDCl₃) δ=0.75 (12H, d), 2.44 (2H, brs), 2.46 (6H, s), 2.60 (3H, s), 6.83 (2H, brs), 6.95-6.98 (4H, m), 7.10-7.15 (4H, m), 7.20-7.25 (2H, m), 7.39 (1H, brs), 7.41 (1H, brs), 7.62 (1H, brs), 7.66 (1H, s), 7.68 (1H, brs), 7.72 (2H, brs), 7.76 (1H, s), 7.85 (1H, s)

MS (ESI+) m/Z: 887.3 ([M+H]⁺)

### [Synthesis Method of Compounds (D-2) and (D-3)]

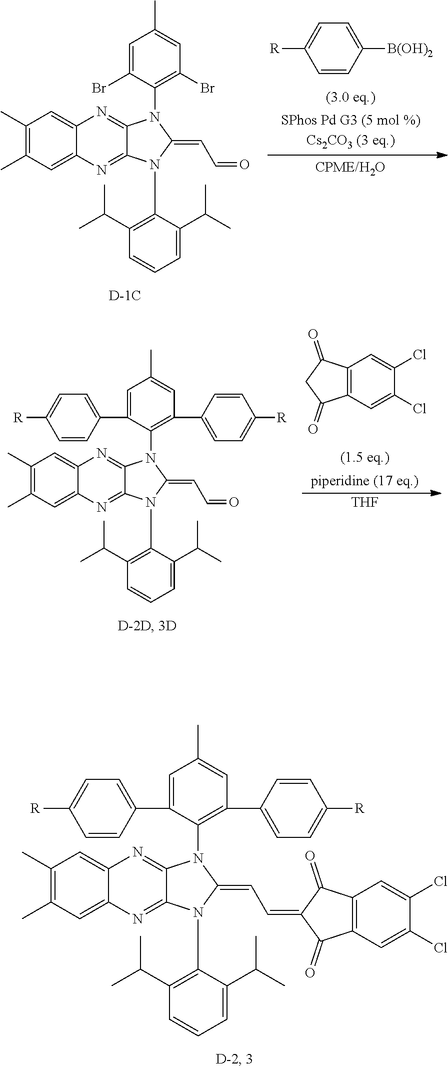

Compounds (D-2) and (D-3) were synthesized by changing the step in the Sonogashira coupling reaction of converting Compound D-1C to Compound D-1D in Compound D-1, to the corresponding Suzuki-Miyaura coupling reaction.

That is, phenylboronic acid having a substituent R was used instead of phenylacetylene, SPhos Pd G3 ((2-dicyclohexylphosphino-2′,6′-dimethoxybiphenyl) [2-(2′-amino-1,1′-biphenyl)]palladium (II) methanesulfonate, CAS No. 1445085-82-4, purchased from Sigma-Aldrich Co. LLC) was used instead of copper (I) iodide and tetrakis(triphenylphosphine) palladium as a catalyst, cesium carbonate was used instead of diisopropylethylamine as a base, and a mixed solvent of cyclopentylmethyl ether (CPME) and water (9:1 volume ratio) was used instead of N,N-dimethylformamide as a solvent.

Compounds D-2D and D-3D were obtained by such a Suzuki-Miyaura coupling reaction, and conversion to Compounds (D-2) and (D-3) were performed in the same manner as Compound (D-1).

### [Synthesis Method of Compounds (D-4) to (D-12)]

Compounds (D-4) to (D-12) were synthesized according to the above-described synthesis method of the Compounds (D-1) to (D-3).

The specific compound and comparative compound used in a test are shown below.

Hereinafter, Compounds (D-1) to (D-12) are specific compounds.

Hereinafter, the specific compound and the comparative compound are collectively referred to as Evaluation Compound.

Evaluation compounds were used for producing photoelectric conversion elements described later.

### <Specific Compound>

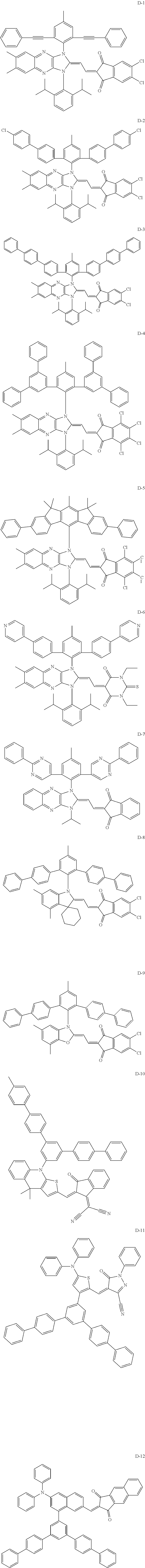

### <Comparative Compound>

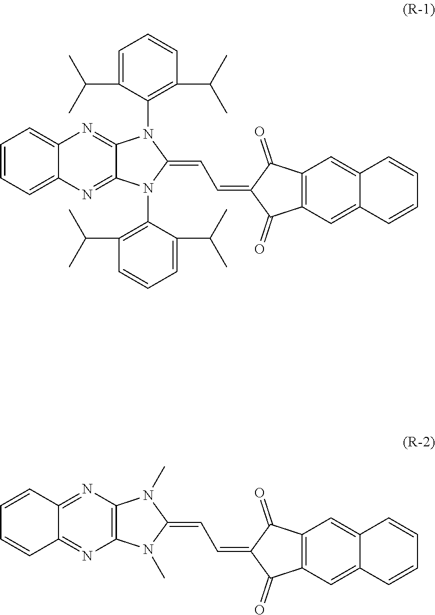 [SMILES: CC(C)c1cccc(C(C)C)c1N1C(=CC=C2C(=O)c3cc4ccccc4cc3C2=O)N(c2c(C(C)C)cccc2C(C)C)c2nc3ccccc3nc21 ; CN1C(=CC=C2C(=O)c3cc4ccccc4cc3C2=O)N(C)c2nc3ccccc3nc21]

[p-Type Semiconductor]

The p-type semiconductor described below was used for producing the photoelectric conversion elements described later, as the p-type semiconductor used for evaluations.

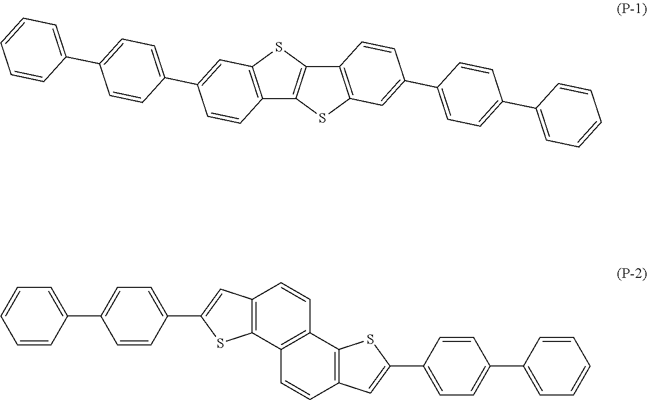 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

[n-Type Semiconductor]

Fullerene C₆₀ (hereinafter, referred to as “C60”) was used for the production of photoelectric conversion elements described later, as the n-type semiconductor used for evaluations.

### [Evaluation]

### [Production of Photoelectric Conversion Element (A)]

A photoelectric conversion element having the form shown in FIG. 2 was produced using evaluation compounds (specific compounds and comparative compounds). Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, a compound (C-1) described below was formed into a film on the lower electrode 11 by a vacuum thermal vapor deposition method to form the electron blocking film 16A (thickness: 30 nm). Furthermore, in a state in which the temperature of the substrate was controlled to 25° C., the evaluation compound shown in Table 4, an n-type semiconductor material, and a p-type semiconductor material (the p-type semiconductor material was optionally added) were co-deposited on the electron blocking films 16A by a vacuum deposition method to form a film having a thickness of 80 nm in terms of a single layer. As a result, a photoelectric conversion film 12 having a bulk hetero structure of 160 nm (240 nm in a case where the p-type semiconductor material was also used) was formed. In this case, a film formation rate of the photoelectric conversion film 12 was set to 1.0 Å/sec. Furthermore, a compound (C-2) described below was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film)(thickness: 10 nm). A SiO film was formed, as a sealing layer, on the upper electrode 15 by a vacuum vapor deposition method, and thereafter, an aluminum oxide (Al₂O₃) layer is formed on the SiO film by an atomic layer chemical vapor deposition (ALCVD) method to produce a photoelectric conversion element.

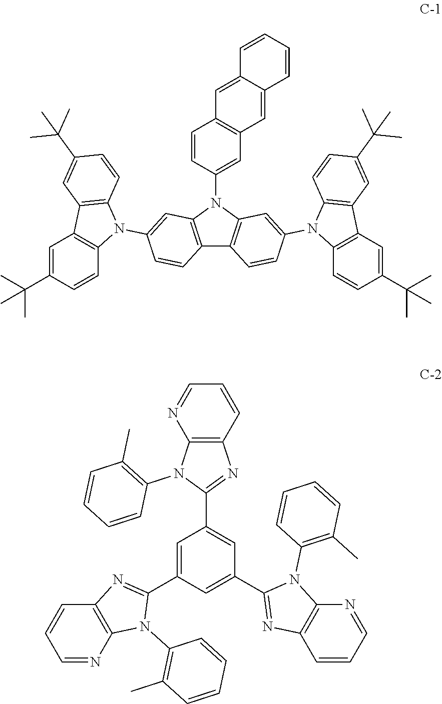 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### [Evaluation 1: Evaluation of Photoelectric Conversion Efficiency (External Quantum Efficiency)]

The driving of each of the photoelectric conversion elements of examples and comparative examples produced by the procedure described in [Production of photoelectric conversion element (A)] was confirmed.

A voltage was applied to each photoelectric conversion element to have an electric field strength of 2.0×10⁵ V/cm. Thereafter, light is emitted from the upper electrode (transparent conductive film) side to perform an incident photon-to-current conversion efficiency (IPCE) measurement, and the photoelectric conversion efficiency (external quantum efficiency) at a wavelength of 560 nm was extracted. The photoelectric conversion efficiency was measured using a constant energy quantum efficiency measuring device manufactured by Optel Co., Ltd. The amount of light emitted was 50 μW/cm². Next, the photoelectric conversion efficiency of each photoelectric conversion element was obtained in a case where the photoelectric conversion efficiency of the photoelectric conversion element of Example 1 was standardized to 1. Then, evaluation was performed according to the following evaluation standard based on the obtained photoelectric conversion efficiency. The results are shown in Table 4. In the following evaluations, from a practical viewpoint, “C” or more is preferable, and “AA” is particularly preferable.

### (Evaluation Standard)

“AA”: 1.1 or more
“A”: 0.9 or more and less than 1.1
“B”: 0.8 or more and less than 0.9
“C”: 0.7 or more and less than 0.8
“D”: 0.6 or more and less than 0.7
“E”: less than 0.6

In addition, it was confirmed that all of the photoelectric conversion elements of Examples and Comparative Examples, which were produced according to the procedure described in [Production of photoelectric conversion element (A)], exhibited a photoelectric conversion efficiency of 40% or more at a measurement wavelength of 560 nm, and the photoelectric conversion elements had an external quantum efficiency of a certain level or higher.

### [Evaluation 2: Evaluation of Responsiveness]

The responsiveness of each of the photoelectric conversion elements of examples and comparative examples produced by the procedure described in [Production of photoelectric conversion element (A)] was evaluated.

A voltage was applied to each photoelectric conversion element to have a strength of 2.0×10⁵ V/cm. Thereafter, light emitting diodes (LED) were turned on momentarily to emit light from the upper electrode (transparent conductive film) side, a photocurrent at a wavelength of 560 nm was measured with an oscilloscope, and a rise time from a signal intensity of 0% (when the light is not emitted) to 97% was calculated. Next, the rise time of each photoelectric conversion element at a wavelength of 560 nm was obtained in a case where the rise time of the photoelectric conversion element of Example 1 was standardized as 1. Then, the responsiveness of each photoelectric conversion element was evaluated based on the obtained rise time according to the following evaluation standard. The results are shown in Table 4. In the following evaluations, from a practical viewpoint, “C” or more is preferable, and “AA” is particularly preferable.

### (Evaluation Standard)

“AA”: less than 0.9
“A”: 0.9 or more and less than 2.0
“B”: 2.0 or more and less than 3.0
“C”: 3.0 or more and less than 4.0
“D”: 4.0 or more and less than 5.0
“E”: less than 5.0

### [Production of Photoelectric Conversion Element (B)]

Each of the photoelectric conversion elements (B) of Examples and Comparative Examples was produced according to the same procedure as [Production of photoelectric conversion element (A)], except that a film formation rate of the photoelectric conversion film 12 was set to 3.0 Å/sec.

### [Evaluation 3: Evaluation of Manufacturing Suitability (Photoelectric Conversion Efficiency During High-Speed Film Formation)]

Using each of the photoelectric conversion elements of Examples and Comparative Examples, which were produced by the procedure described in [Production of photoelectric conversion element (B)], the photoelectric conversion efficiency (external quantum efficiency) was obtained by the method described in [Evaluation 1: evaluation of photoelectric conversion efficiency (external quantum efficiency)].

Next, the film formation rate dependence was calculated by Expression (X) for each of the photoelectric conversion elements of Examples and Comparative Examples, and the manufacturing suitability of each photoelectric conversion element was evaluated based on the following evaluation standard. The results are shown in Table 4. The film formation rate dependence of Example 1 was calculated by dividing the photoelectric conversion efficiency of the photoelectric conversion element of Example 1 obtained by the production method of the photoelectric conversion element (B) by the photoelectric conversion efficiency of the photoelectric conversion element of Example 1 obtained by the production method of the photoelectric conversion element (A).

### Expression (X):

Film formation rate dependence=photoelectric conversion efficiency of photoelectric conversion element (B)/photoelectric conversion efficiency of photoelectric conversion element (A)

### (Evaluation Standard)

“A”: 0.95 or more
“B”: less than 0.95

Hereinafter, Table 4 is shown.

The column of “Remarks” in the tables indicates which of the compound represented by any of Formulae (1) to (6) correspond to the evaluation compound.

TABLE 4

| | | | | | Evaluation results | | |
|---|---|---|---|---|---|---|---|
| | Main component of photoelectric | | | | Evaluation 1 | | |
| | conversion film | | | | Photoelectric | | Evaluation 3 |
| | Evaluation | n-Type organic | p-Type organic | | conversion | Evaluation 2 | Manufacturing |
| | Compound | semiconductor | semiconductor | Remarks | efficiency | Responsiveness | suitability |
| Example 1-1 | (D-1) | C₆₀ | — | Corresponding | A | A | A |
| | | | | to Formula (1) | | | |
| Example 1-2 | (D-2) | C₆₀ | — | Corresponding | A | A | A |
| | | | | to Formula (1) | | | |
| Example 1-3 | (D-3) | C₆₀ | — | Corresponding | A | A | A |
| | | | | to Formula (1) | | | |
| Example1-4 | (D-4) | C₆₀ | — | Corresponding | A | A | A |
| | | | | to Formula (1) | | | |
| Example 1-5 | (D-5) | C₆₀ | — | Corresponding | A | A | A |
| | | | | to Formula (1) | | | |
| Example 1-6 | (D-6) | C₆₀ | — | Corresponding | A | A | A |
| | | | | to Formula (1) | | | |
| Example 1-7 | (D-7) | C₆₀ | — | Corresponding | A | A | A |
| | | | | to Formula (1) | | | |
| Example 1-8 | (D-8) | C₆₀ | — | Corresponding | A | A | A |
| | | | | to Formula (2) | | | |
| Example 1-9 | (D-9) | C₆₀ | — | Corresponding | A | A | A |
| | | | | to Formula (3) | | | |
| Example 1-10 | (D-10) | C₆₀ | — | Corresponding | A | A | A |
| | | | | to Formula (4) | | | |
| Example 1-11 | (D-11) | C₆₀ | — | Corresponding | A | A | A |
| | | | | to Formula (5) | | | |
| Example 1-12 | (D-12) | C₆₀ | — | Corresponding | A | A | A |
| | | | | to Formula (6) | | | |
| Example 1-13 | (D-1) | C₆₀ | (P-1) | Corresponding | AA | AA | A |
| | | | | to Formula (1) | | | |
| Example 1-14 | (D-1) | C₆₀ | (P-2) | Corresponding | AA | AA | A |
| | | | | to Formula (1) | | | |
| Comparative | (R-1) | C₆₀ | — | — | A | B | B |
| Example 1-1 | | | | | | | |
| Comparative | (R-2) | C₆₀ | — | — | A | B | B |
| Example 1-2 | | | | | | | |

From the results in Table 4, it was found that the photoelectric conversion elements of Examples had excellent manufacturing suitability. In addition, it was found that the photoelectric conversion elements of Examples were also excellent in responsiveness and external quantum efficiency.

On the other hand, it was confirmed that the desired effect was not obtained in the photoelectric conversion elements of Comparative Examples.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film
20a: Imaging element
22: blue photoelectric conversion element
24: red photoelectric conversion element

What is claimed is:

1. A photoelectric conversion element comprising, in the following order:
a conductive film;
a photoelectric conversion film; and
a transparent conductive film,
wherein the photoelectric conversion film contains one or more of compounds represented by any of Formulae (1) to (6), 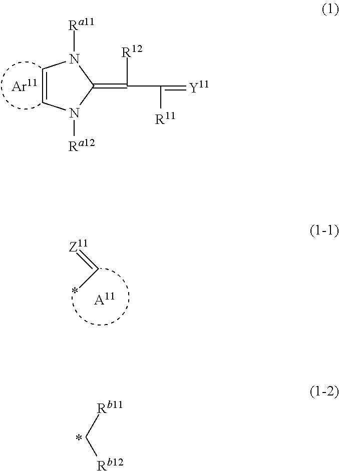
in Formula (1), Y¹¹ represents a group represented by Formula (1-1) or a group represented by Formula (1-2),
A¹¹ represents a ring which contains at least two carbon atoms and may have a substituent,
Z¹¹ represents an oxygen atom, a sulfur atom, —NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b11} and R^{b12} each independently represent a cyano group or —COOR^{B1}, and R^{B1} represents an alkyl group which may have a substituent, an aryl group which may have a substituent, or a heteroaryl group which may have a substituent,
* represents a bonding position,
R¹¹ and R¹² each independently represent a hydrogen atom or a substituent,
R^{a11} and R^{a12} each independently represent an aromatic ring group which may have a substituent, or —C(R^{L11})(R^{L12})(R^{L13}), R^{L11} represents a hydrogen atom, an alkyl group which may have a substituent, an aryl group which may have a substituent, or a heteroaryl group which may have a substituent, R^{L12} and R^{L13} each independently represent an alkyl group which may have a substituent, an aryl group which may have a substituent, or a heteroaryl group which may have a substituent, and R^{L11} to R^{L13} may be bonded to each other to form a ring, and
Ar¹¹ represents an aromatic ring which contains at least two carbon atoms and may have a substituent,
provided that in Formula (1), at least one of R^{a11} or R^{a12} represents an aromatic ring group represented by Formula (X), 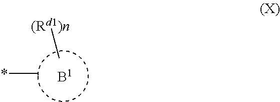
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position, 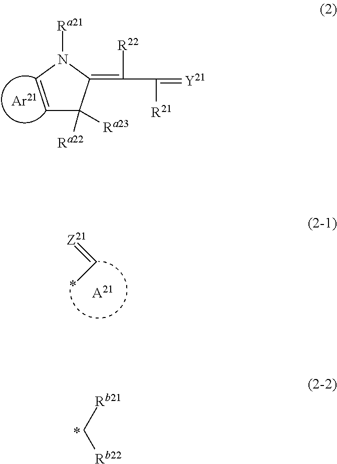
in Formula (2), Y²¹ represents a group represented by Formula (2-1) or a group represented by Formula (2-2),
A²¹ represents a ring which contains at least two carbon atoms and may have a substituent,
Z²¹ represents an oxygen atom, a sulfur atom, =NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b21} and R^{b22} each independently represent a cyano group or —COOR^{B1}, and R^{B1} represents an alkyl group which may have a substituent, an aryl group which may have a substituent, or a heteroaryl group which may have a substituent,
* represents a bonding position,
R²¹ and R²² each independently represent a hydrogen atom or a substituent,
Ar²¹ represents an aromatic ring which contains at least two carbon atoms and may have a substituent, and
R^{a21} to R^{a23} each independently represent an alkyl group which may have a substituent or an aromatic ring group which may have a substituent, and R^{a22} and R^{a23} may be bonded to each other to form a ring, provided that at least one of R^{a21}, R^{a22}, or R^{a23} represents an aromatic ring group represented by Formula (X), 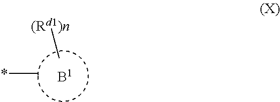
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position, 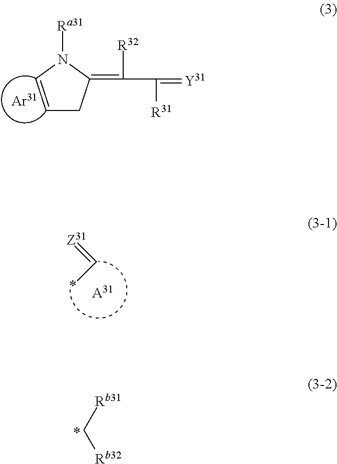
in Formula (3), Y³¹ represents a group represented by Formula (3-1) or a group represented by Formula (3-2),
A³¹ represents a ring which contains at least two carbon atoms and may have a substituent,
Z³¹ represents an oxygen atom, a sulfur atom, =NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b31} and R^{b32} each independently represent a cyano group or —COOR^{B1}, and R^{B1} represents an alkyl group which may have a substituent, an aryl group which may have a substituent, or a heteroaryl group which may have a substituent,
* represents a bonding position,
R³¹ and R³² each independently represent a hydrogen atom or a substituent,
Ar³¹ represents an aromatic ring which contains at least two carbon atoms and may have a substituent,
X³¹ represents an oxygen atom or a sulfur atom, and
R^{a31} represents an aromatic ring group represented by Formula (X), 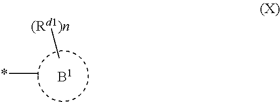
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position, 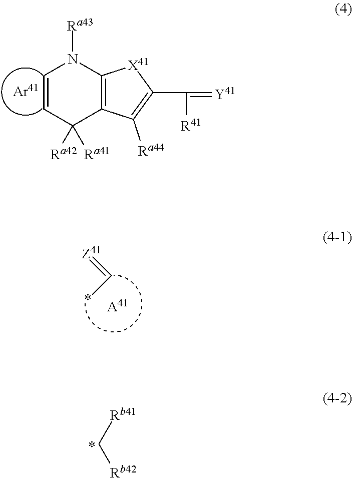
in Formula (4), Y⁴¹ represents a group represented by Formula (4-1) or a group represented by Formula (4-2),
A⁴¹ represents a ring which contains at least two carbon atoms and may have a substituent,
Z⁴¹ represents an oxygen atom, a sulfur atom, =NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b41} and R^{b42} each independently represent a cyano group or —COOR^{B1}, and R^{B1} represents an alkyl group which may have a substituent, an aryl group which may have a substituent, or a heteroaryl group which may have a substituent,
* represents a bonding position,
R⁴¹ represents a hydrogen atom or a substituent,
Ar⁴¹ represents an aromatic ring which contains at least two carbon atoms and may have a substituent,
X⁴¹ represents an oxygen atom, a sulfur atom, —NR^{CT1}—, —CR^{CT2}R^{CT3}—, or —C(R^{CT4})—C(R^{CT5})—, and R^{CT1} to R^{CT5} each independently represent a hydrogen atom or a substituent,
R^{a41} to R^{a44} each independently represent a hydrogen atom, an alkyl group which may have a substituent, or an aromatic ring group which may have a substituent, and R^{a41} and R^{a42} may be bonded to each other to form a ring, provided that at least one of R^{a41}, . . . , or R^{a44} represents an aromatic ring group represented by Formula (X), 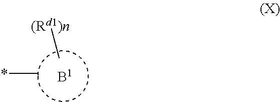
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position, 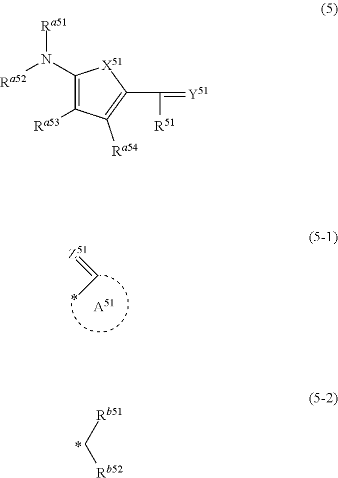
in Formula (5), Y⁵¹ represents a group represented by Formula (5-1) or a group represented by Formula (5-2),
A⁵¹ represents a ring which contains at least two carbon atoms and may have a substituent,
Z⁵¹ represents an oxygen atom, a sulfur atom, —NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b51} and R^{b52} each independently represent a cyano group or —COOR^{B1}, and R^{B1} represents an alkyl group which may have a substituent, an aryl group which may have a substituent, or a heteroaryl group which may have a substituent,
* represents a bonding position,
R⁵¹ represents a hydrogen atom or a substituent,
X⁵¹ represents an oxygen atom, a sulfur atom, —NR^{CT1}—, —CR^{CT2}R^{CT3}—, or —C(R^{CT4})═C(R^{CT5})—, and R^{CT1} to R^{CT5} each independently represent a hydrogen atom or a substituent,
R^{a51} and R^{a52} each independently represent an alkyl group which may have a substituent or an aromatic ring group which may have a substituent, and R^{a53} and R^{a54} each independently represent a hydrogen atom, an alkyl group which may have a substituent, or an aromatic ring group which may have a substituent, provided that at least one of R^{a51}, . . . , or R^{a54} represents an aromatic ring group represented by Formula (X), 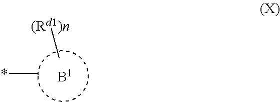
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position, 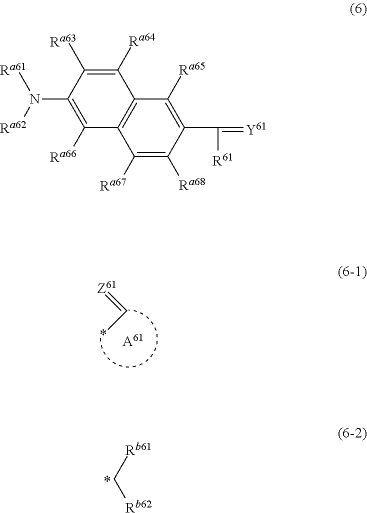
in Formula (6), Y⁶¹ represents a group represented by Formula (6-1) or a group represented by Formula (6-2),
A⁶¹ represents a ring which contains at least two carbon atoms and may have a substituent,
Z⁶¹ represents an oxygen atom, a sulfur atom, =NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b61} and R^{b62} each independently represent a cyano group or —COOR^{B1}, and R^{B1} represents an alkyl group which may have a substituent, an aryl group which may have a substituent, or a heteroaryl group which may have a substituent,
* represents a bonding position,
R⁶¹ represents a hydrogen atom or a substituent,
R^{a61} and R^{a62} each independently represent an alkyl group which may have a substituent or an aromatic ring group which may have a substituent, and R^{a63} to R^{a68} each independently represent a hydrogen atom, an alkyl group which may have a substituent, or an aromatic ring group which may have a substituent, provided that at least one of R^{a61}, . . . , or R^{a68} represents an aromatic ring group represented by Formula (X), 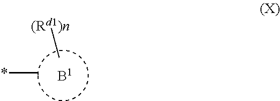
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position.

2. The photoelectric conversion element according to claim 1,
wherein the compound represented by Formula (1) is a compound represented by Formula (1A), 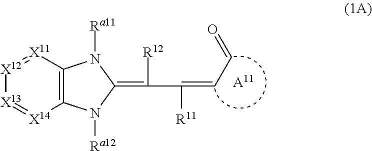
in Formula (1A), A¹¹ represents a ring which contains at least two carbon atoms and may have a substituent,
R¹¹ and R¹² each independently represent a hydrogen atom or a substituent,
X¹¹ to X¹⁴ each independently represent a nitrogen atom or —CR^{c11}=, R^{c11} represents a hydrogen atom or a substituent, and in a case where a plurality of R^{c11}'s are present, the plurality of R^{c11}'s may be bonded to each other to form a ring, and
R^{a11} and R^{a12} each independently represent an aromatic ring group which may have a substituent, or —C(R^{L11})(R^{L12})(R^{L13}), R^{L11} represents a hydrogen atom, an alkyl group which may have a substituent, an aryl group which may have a substituent, or a heteroaryl group which may have a substituent, R^{L12} and R^{L13} each independently represent an alkyl group which may have a substituent, an aryl group which may have a substituent, or a heteroaryl group which may have a substituent, and R^{L11} to R^{L13} may be bonded to each other to form a ring,
provided that in Formula (1A), at least one of R^{a11} or R^{a12} represents an aromatic ring group represented by Formula (X), 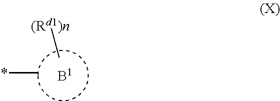
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position.

3. The photoelectric conversion element according to claim 1,
wherein the compound represented by Formula (1) is a compound represented by Formula (1B), 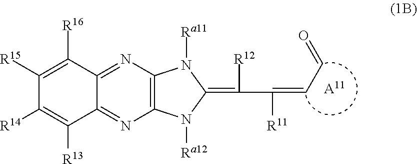
in Formula (1B), A¹¹ represents a ring which contains at least two carbon atoms and may have a substituent,
R¹¹ to R¹⁶ each independently represent a hydrogen atom or a substituent, R¹³ and R¹⁴, R¹⁴ and R¹⁵, and R¹⁵ and R¹⁶ each may be independently bonded to each other to form a ring, and
R^{a11} and R^{a12} each independently represent an aromatic ring group which may have a substituent, or —C(R^{L11})(R^{L12})(R^{L13}), R^{L11} represents a hydrogen atom, an alkyl group which may have a substituent, an aryl group which may have a substituent, or a heteroaryl group which may have a substituent, R^{L12} and R^{L13} each independently represent an alkyl group which may have a substituent, an aryl group which may have a substituent, or a heteroaryl group which may have a substituent, and R^{L11} to R^{L13} may be bonded to each other to form a ring,
provided that in Formula (1B), at least one of R^{a11} or R^{a12} represents an aromatic ring group represented by Formula (X), 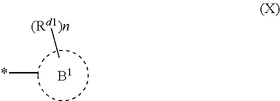
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position.

4. The photoelectric conversion element according to claim 2,
wherein the compound represented by Formula (1) is a compound represented by Formula (1C), 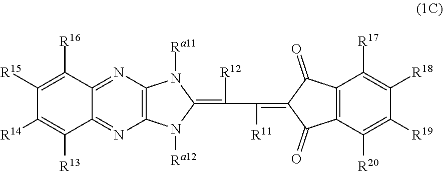
in Formula (1C), R¹¹ to R²⁰ each independently represent a hydrogen atom or a substituent, and R¹³ and R¹⁴, R¹⁴ and R¹⁵, R¹⁵ and R¹⁶, R¹⁷ and R¹⁸, R¹⁸ and R¹⁹, and R¹⁹ and R²⁰ each may be independently bonded to each other to form a ring, and
R^{a11} and R^{a12} each independently represent an aromatic ring group which may have a substituent, or —C(R^{L11})(R^{L12})(R^{L13}), R^{L11} represents a hydrogen atom, an alkyl group which may have a substituent, an aryl group which may have a substituent, or a heteroaryl group which may have a substituent, R^{L12} and R^{L13} each independently represent an alkyl group which may have a substituent, an aryl group which may have a substituent, or a heteroaryl group which may have a substituent, and R^{L11} to R^{L13} may be bonded to each other to form a ring,
provided that in Formula (1C), at least one of R^{a11} or R^{a12} represents an aromatic ring group represented by Formula (X), 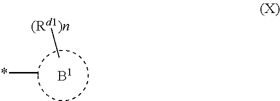
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position.

5. The photoelectric conversion element according to claim 1,
wherein the photoelectric conversion film contains one or more of the compounds represented by any of Formulae (1) to (3).

6. The photoelectric conversion element according to claim 1,
wherein the aromatic ring group represented by Formula (X) represents an aromatic ring group represented by Formula (X′), 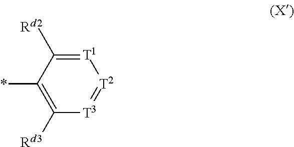
in Formula (X′), T¹ to T³ each independently represent —CR^{e12}═ or a nitrogen atom, and R^{e12} represents a hydrogen atom or a substituent,
R^{d2} and R^{d3} each independently represent an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
in a case where T¹ represents —CR^{e12}—, R^{d2} and R^{e12} may be bonded to each other to form a non-aromatic ring, and in a case where T³ represents —CR^{e12}=, R^{d3} and R^{e12} may be bonded to each other to form a non-aromatic ring, and
* represents a bonding position.

7. The photoelectric conversion element according to claim 1,
wherein the photoelectric conversion film further contains an n-type organic semiconductor, and
the photoelectric conversion film has a bulk hetero structure formed in a state where one or more of the compounds represented by any of Formulae (1) to (6) and the n-type organic semiconductor are mixed with each other.

8. The photoelectric conversion element according to claim 7,
wherein the n-type organic semiconductor includes fullerenes selected from the group consisting of a fullerene and a derivative of a fullerene.

9. The photoelectric conversion element according to claim 1, further comprising:
one or more interlayers between the conductive film and the transparent conductive film, in addition to the photoelectric conversion film.

10. An imaging element comprising:
the photoelectric conversion element according to claim 1.

11. The imaging element according to claim 10, further comprising:
another photoelectric conversion element that receives light having a wavelength different from a wavelength of light received by the photoelectric conversion element.

12. The imaging element according to claim 11,
wherein the photoelectric conversion element and the other photoelectric conversion element are laminated, and
at least a part of incidence ray is transmitted through the photoelectric conversion element, and then is received by the other photoelectric conversion element.

13. The imaging element according to claim 11,
wherein the photoelectric conversion element is a green photoelectric conversion element, and
the other photoelectric conversion element includes a blue photoelectric conversion element and a red photoelectric conversion element.

14. An optical sensor comprising:
the photoelectric conversion element according to claim 1.

15. A compound represented by Formula (1), 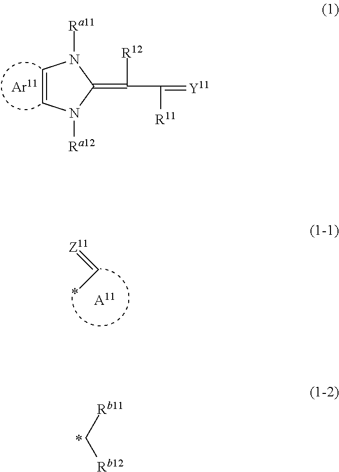
in Formula (1), Y¹¹ represents a group represented by Formula (1-1) or a group represented by Formula (1-2),
A¹¹ represents a ring which contains at least two carbon atoms and may have a substituent,
Z¹¹ represents an oxygen atom, a sulfur atom, =NR^{ZT1}, or =CR^{ZT2}R^{ZT3}, R^{ZT1} represents a hydrogen atom or a substituent, R^{ZT2} and R^{ZT3} each independently represent a cyano group or —COOR^{ZT4}, and R^{ZT4} represents an alkyl group which may have a substituent, an aryl group which may have a substituent, or a heteroaryl group which may have a substituent,
R^{b11} and R^{b12} each independently represent a cyano group or —COOR^{B1}, and R^{B1} represents an alkyl group which may have a substituent, an aryl group which may have a substituent, or a heteroaryl group which may have a substituent,
* represents a bonding position,
R¹¹ and R¹² each independently represent a hydrogen atom or a substituent,
R^{a11} and R^{a12} each independently represent an aromatic ring group which may have a substituent, or —C(R^{L11})(R^{L12})(R^{L13}), R^{L11} represents a hydrogen atom, an alkyl group which may have a substituent, an aryl group which may have a substituent, or a heteroaryl group which may have a substituent, R^{L12} and R^{L13} each independently represent an alkyl group which may have a substituent, an aryl group which may have a substituent, or a heteroaryl group which may have a substituent, and R^{L11} to R^{L13} may be bonded to each other to form a ring, and
Ar¹¹ represents an aromatic ring which contains at least two carbon atoms and may have a substituent,
provided that in Formula (1), at least one of R^{a11} or R^{a12} represents an aromatic ring group represented by Formula (X), 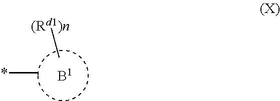
in Formula (X), B¹ represents an aromatic ring which may have a substituent other than R^{d1},
R^{d1} represents an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
the substituent which may be contained in B¹ and R^{d1} may be bonded to each other to form a non-aromatic ring, and R^{d1}'s may be bonded to each other to form a non-aromatic ring,
a plurality of R^{d1}'s present in Formula (X) may be the same or different from each other,
n represents an integer of 2 or 3, and
* represents a bonding position.

16. The photoelectric conversion element according to claim 2,
wherein the aromatic ring group represented by Formula (X) represents an aromatic ring group represented by Formula (X′), 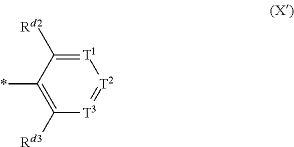
in Formula (X′), T¹ to T³ each independently represent —CR^{e12}═ or a nitrogen atom, and R^{e12} represents a hydrogen atom or a substituent,
R^{d2} and R^{d3} each independently represent an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
in a case where T¹ represents —CR^{e12}=, R^{d2} and R^{e12} may be bonded to each other to form a non-aromatic ring, and in a case where T³ represents —CR^{e12}=, R^{d3} and R^{e12} may be bonded to each other to form a non-aromatic ring, and
* represents a bonding position.

17. The photoelectric conversion element according to claim 3,
wherein the aromatic ring group represented by Formula (X) represents an aromatic ring group represented by Formula (X′), 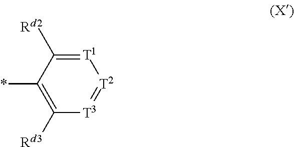
in Formula (X′), T¹ to T³ each independently represent —CR^{e12}═ or a nitrogen atom, and R^{e12} represents a hydrogen atom or a substituent,
R^{d2} and R^{d3} each independently represent an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
in a case where T¹ represents —CR^{e12}=, R^{d2} and R^{e12} may be bonded to each other to form a non-aromatic ring, and in a case where T³ represents —CR^{e12}=, R^{d3} and R^{e12} may be bonded to each other to form a non-aromatic ring, and
* represents a bonding position.

18. The photoelectric conversion element according to claim 4,
wherein the aromatic ring group represented by Formula (X) represents an aromatic ring group represented by Formula (X′), 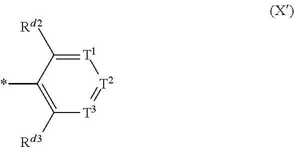
in Formula (X′), T¹ to T³ each independently represent —CR^{e12}═ or a nitrogen atom, and R^{e12} represents a hydrogen atom or a substituent,
R^{d2} and R^{d3} each independently represent an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
in a case where T¹ represents —CR^{e12}=, R^{d2} and R^{e12} may be bonded to each other to form a non-aromatic ring, and in a case where T³ represents —CR^{e12}=, R^{d3} and R^{e12} may be bonded to each other to form a non-aromatic ring, and
* represents a bonding position.

19. The photoelectric conversion element according to claim 5,
wherein the aromatic ring group represented by Formula (X) represents an aromatic ring group represented by Formula (X′), 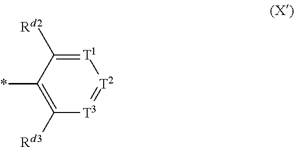
in Formula (X′), T¹ to T³ each independently represent —CR^{e12}═ or a nitrogen atom, and R^{e12} represents a hydrogen atom or a substituent,
R^{d2} and R^{d3} each independently represent an aryl group having a substituent A, a heteroaryl group having the substituent A, an alkenyl group having the substituent A, or an alkynyl group having the substituent A, and the substituent A represents an aryl group which may have a substituent or a heteroaryl group which may have a substituent,
in a case where T¹ represents —CR^{e12}=, R^{d2} and R^{e12} may be bonded to each other to form a non-aromatic ring, and in a case where T³ represents —CR^{e12}=, R^{d3} and R^{e12} may be bonded to each other to form a non-aromatic ring, and
* represents a bonding position.
